SEQUENCE LISTING (342 sequences)

SEQ ID NO: 1 - protein, 8 aa, synthetic construct
  VARIANT 2: note = V or I
  VARIANT 4: note = Any amino acid
DXEXNPGP

SEQ ID NO: 2 - protein, 8 aa, synthetic construct
SIINFEKL

SEQ ID NO: 3 - protein, 11 aa, synthetic construct
SIINFEKLAAY

SEQ ID NO: 4 - protein, 20 aa, synthetic construct
GLKGPDIYKGVYQFKSVEFD

SEQ ID NO: 5 - protein, 61 aa, synthetic construct
  VARIANT 35: note = V or I
  VARIANT 37: note = Any amino acid
SIINFEKLAAYSIINFEKLAAYSIINFEKLAAYDXEXNPGPGLKGPDIYKGVYQFKSVEFD

SEQ ID NO: 6 - protein, 33 aa, synthetic construct
SIINFEKLAAYSIINFEKLAAYSIINFEKLAAY

SEQ ID NO: 7 - protein, 9 aa, synthetic construct
AEPINIQTW

SEQ ID NO: 8 - protein, 9 aa, synthetic construct
FPSDSWCYF

SEQ ID NO: 9 - protein, 9 aa, synthetic construct
SYLDSGIHF

SEQ ID NO: 10 - protein, 10 aa, synthetic construct
ACDPHSGHFV

SEQ ID NO: 11 - protein, 10 aa, synthetic construct
AVCPWTWLRG

SEQ ID NO: 12 - protein, 9 aa, synthetic construct
ILDKVLVHL

SEQ ID NO: 13 - protein, 10 aa, synthetic construct
TLDWLLQTPK

SEQ ID NO: 14 - protein, 9 aa, synthetic construct
RPHVPESAF

SEQ ID NO: 15 - protein, 9 aa, synthetic construct
KIFSEVTLK

SEQ ID NO: 16 - protein, 9 aa, synthetic construct
SHETVIIEL

SEQ ID NO: 17 - protein, 10 aa, synthetic construct
KILDAVVAQK

SEQ ID NO: 18 - protein, 11 aa, synthetic construct
FLEGNEVGKTY

SEQ ID NO: 19 - protein, 9 aa, synthetic construct
EEKLIVVLF

SEQ ID NO: 20 - protein, 11 aa, synthetic construct
SELFRSGLDSY

SEQ ID NO: 21 - protein, 9 aa, synthetic construct
FRSGLDSYV

SEQ ID NO: 22 - protein, 9 aa, synthetic construct
EAFIQPITR

SEQ ID NO: 23 - protein, 9 aa, synthetic construct
KINKNPKYK

SEQ ID NO: 24 - protein, 10 aa, synthetic construct
ILDTAGREEY

SEQ ID NO: 25 - protein, 9 aa, synthetic construct
KELEGILLL

SEQ ID NO: 26 - protein, 9 aa, synthetic construct
ETVSEQSNV

SEQ ID NO: 27 - protein, 8 aa, synthetic construct
QQITKTEV

SEQ ID NO: 28 - protein, 10 aa, synthetic construct
FIASNGVKLV

SEQ ID NO: 29 - protein, 9 aa, synthetic construct
FLDEFMEGV

SEQ ID NO: 30 - protein, 10 aa, synthetic construct
SLFEGIDIYT

SEQ ID NO: 31 - protein, 9 aa, synthetic construct
VESEDIAEL

SEQ ID NO: 32 - protein, 9 aa, synthetic construct
RTKVVQTLW

SEQ ID NO: 33 - protein, 9 aa, synthetic construct
IPIDGIFFT

SEQ ID NO: 34 - protein, 9 aa, synthetic construct
KMIGNHLWV

SEQ ID NO: 35 - protein, 9 aa, synthetic construct
AMFWSVPTV

SEQ ID NO: 36 - protein, 9 aa, synthetic construct
CLNEYHLFL

SEQ ID NO: 37 - protein, 9 aa, synthetic construct
KLMNIQQKL

SEQ ID NO: 38 - protein, 9 aa, synthetic construct
QLSCISTYV

SEQ ID NO: 39 - protein, 9 aa, synthetic construct
FLYNLLTRV

SEQ ID NO: 40 - protein, 9 aa, synthetic construct
IILVAVPHV

SEQ ID NO: 41 - protein, 9 aa, synthetic construct
HLYASLSRA

SEQ ID NO: 42 - protein, 9 aa, synthetic construct
MLGEQLFPL

SEQ ID NO: 43 - protein, 10 aa, synthetic construct
RLFPGLTIKI

SEQ ID NO: 44 - protein, 10 aa, synthetic construct
TRSSGSHFVF

SEQ ID NO: 45 - protein, 9 aa, synthetic construct
LRTKVYAEL

SEQ ID NO: 46 - protein, 9 aa, synthetic construct
LLYQELLPL

SEQ ID NO: 47 - protein, 9 aa, synthetic construct
IYKAPCENW

SEQ ID NO: 48 - protein, 10 aa, synthetic construct
YYPPSQIAQL

SEQ ID NO: 49 - protein, 9 aa, synthetic construct
LYNGMEHLI

SEQ ID NO: 50 - protein, 9 aa, synthetic construct
HSVSSAFKK

SEQ ID NO: 51 - protein, 9 aa, synthetic construct
YPPPPPALL

SEQ ID NO: 52 - protein, 9 aa, synthetic construct
KVDPIGHVY

SEQ ID NO: 53 - protein, 11 aa, synthetic construct
LMKVDPIGHVY

SEQ ID NO: 54 - protein, 10 aa, synthetic construct
KVDPIGHVYF

SEQ ID NO: 55 - protein, 10 aa, synthetic construct
FVVPYMIYLL

SEQ ID NO: 56 - protein, 10 aa, synthetic construct
VSVQIISCQY

SEQ ID NO: 57 - protein, 8 aa, synthetic construct
VQIISCQY

SEQ ID NO: 58 - protein, 9 aa, synthetic construct
CVRVSGQGL

SEQ ID NO: 59 - protein, 11 aa, synthetic construct
APARLERRHSA

SEQ ID NO: 60 - protein, 9 aa, synthetic construct
AYHSIEWAI

SEQ ID NO: 61 - protein, 8 aa, synthetic construct
YHSIEWAI

SEQ ID NO: 62 - protein, 10 aa, synthetic construct
NAYHSIEWAI

SEQ ID NO: 63 - protein, 9 aa, synthetic construct
KSQREFVRR

SEQ ID NO: 64 - protein, 9 aa, synthetic construct
MRMNQGVCC

SEQ ID NO: 65 - protein, 9 aa, synthetic construct
FLSDHLYLV

SEQ ID NO: 66 - protein, 10 aa, synthetic construct
KPSDTPRPVM

SEQ ID NO: 67 - protein, 9 aa, synthetic construct
HIAKSLFEV

SEQ ID NO: 68 - protein, 10 aa, synthetic construct
AGQHIAKSLF

SEQ ID NO: 69 - protein, 9 aa, synthetic construct
KPFCVLISL

SEQ ID NO: 70 - protein, 9 aa, synthetic construct
RPHHDQRSL

SEQ ID NO: 71 - protein, 9 aa, synthetic construct
SSYTGFANK

SEQ ID NO: 72 - protein, 9 aa, synthetic construct
FLLDEAIGL

SEQ ID NO: 73 - protein, 10 aa, synthetic construct
ALDPHSGHFV

SEQ ID NO: 74 - protein, 9 aa, synthetic construct
HSCVMASLR

SEQ ID NO: 75 - protein, 9 aa, synthetic construct
HDLGRLHSC

SEQ ID NO: 76 - protein, 9 aa, synthetic construct
VSKILPSTW

SEQ ID NO: 77 - protein, 9 aa, synthetic construct
GVADVLLYR

SEQ ID NO: 78 - protein, 9 aa, synthetic construct
TESPFEQHI

SEQ ID NO: 79 - protein, 9 aa, synthetic construct
GLEREGFTF

SEQ ID NO: 80 - protein, 9 aa, synthetic construct
YTDFHCQYV

SEQ ID NO: 81 - protein, 9 aa, synthetic construct
CILGKLFTK

SEQ ID NO: 82 - protein, 9 aa, synthetic construct
GLFGDIYLA

SEQ ID NO: 83 - protein, 10 aa, synthetic construct
SLADEAEVYL

SEQ ID NO: 84 - protein, 9 aa, synthetic construct
KTLTSVFQK

SEQ ID NO: 85 - protein, 10 aa, synthetic construct
ILNAMIAKIJ

SEQ ID NO: 86 - protein, 9 aa, synthetic construct
FAFQEYDSF

SEQ ID NO: 87 - protein, 8 aa, synthetic construct
LLDIVAPK

SEQ ID NO: 88 - protein, 9 aa, synthetic construct
SPMIVGSPW

SEQ ID NO: 89 - protein, 9 aa, synthetic construct
ASNASSAAK

SEQ ID NO: 90 - protein, 8 aa, synthetic construct
CYMEAVAL

SEQ ID NO: 91 - protein, 15 aa, synthetic construct
TLYQDDTLTLQAAGE

SEQ ID NO: 92 - protein, 16 aa, synthetic construct
YSVYFNLPADTIYTNH

SEQ ID NO: 93 - protein, 13 aa, synthetic construct
FSWAMDLDPKGAE

SEQ ID NO: 94 - protein, 14 aa, synthetic construct
MIFEKHGFRRTTPP

SEQ ID NO: 95 - protein, 9 aa, synthetic construct
WRRAPAPGA

SEQ ID NO: 96 - protein, 9 aa, synthetic construct
PVTWRRAPA

SEQ ID NO: 97 - protein, 12 aa, synthetic construct
RVIKNSIRLTLE

SEQ ID NO: 98 - protein, 16 aa, synthetic construct
PYYFAAELPPRNLPEP

SEQ ID NO: 99 - protein, 15 aa, synthetic construct
GELIGILNAAKVPAD

SEQ ID NO: 100 - protein, 9 aa, synthetic construct
SVGDFSQEF

SEQ ID NO: 101 - protein, 10 aa, synthetic construct
VSVGDFSQEF

SEQ ID NO: 102 - protein, 9 aa, synthetic construct
KLKFVTLVF

SEQ ID NO: 103 - protein, 9 aa, synthetic construct
VLAKKLKFV

SEQ ID NO: 104 - protein, 9 aa, synthetic construct
FLFQDSKKI

SEQ ID NO: 105 - protein, 9 aa, synthetic construct
NSKKKWFLF

SEQ ID NO: 106 - protein, 10 aa, synthetic construct
VQKVASKIPF

SEQ ID NO: 107 - protein, 9 aa, synthetic construct
ALFASRPRF

SEQ ID NO: 108 - protein, 10 aa, synthetic construct
RFLEYLPLRF

SEQ ID NO: 109 - protein, 9 aa, synthetic construct
TELERFLEY

SEQ ID NO: 110 - protein, 9 aa, synthetic construct
LLHTELERF

SEQ ID NO: 111 - protein, 10 aa, synthetic construct
LLHTELERFL

SEQ ID NO: 112 - protein, 9 aa, synthetic construct
TLFHTFYEL

SEQ ID NO: 113 - protein, 10 aa, synthetic construct
TLFHTFYELL

SEQ ID NO: 114 - protein, 10 aa, synthetic construct
LFHTFYELLI

SEQ ID NO: 115 - protein, 9 aa, synthetic construct
LFHTFYELL

SEQ ID NO: 116 - protein, 10 aa, synthetic construct
TTLFHTFYEL

SEQ ID NO: 117 - protein, 9 aa, synthetic construct
KFGDLTNNF

SEQ ID NO: 118 - protein, 9 aa, synthetic construct
KLFESKAEL

SEQ ID NO: 119 - protein, 10 aa, synthetic construct
KLFESKAELA

SEQ ID NO: 120 - protein, 9 aa, synthetic construct
YNSFSSAPM

SEQ ID NO: 121 - protein, 10 aa, synthetic construct
SFSSAPMPQI

SEQ ID NO: 122 - protein, 9 aa, synthetic construct
GIPENSFNV

SEQ ID NO: 123 - protein, 9 aa, synthetic construct
SVGDFSQEF

SEQ ID NO: 124 - protein, 10 aa, synthetic construct
VSVGDFSQEF

SEQ ID NO: 125 - protein, 9 aa, synthetic construct
TPAAPTAMA

SEQ ID NO: 126 - protein, 9 aa, synthetic construct
FPGNQWNPV

SEQ ID NO: 127 - protein, 10 aa, synthetic construct
LADFRLARLY

SEQ ID NO: 128 - protein, 9 aa, synthetic construct
NHDETSFLL

SEQ ID NO: 129 - protein, 9 aa, synthetic construct
TPAHPSQGA

SEQ ID NO: 130 - protein, 10 aa, synthetic construct
TPAHPSQGAV

SEQ ID NO: 131 - protein, 9 aa, synthetic construct
VTEKLQPTY

SEQ ID NO: 132 - protein, 10 aa, synthetic construct
HPAPPAPPPA

SEQ ID NO: 133 - protein, 10 aa, synthetic construct
VPKEHPAPPA

SEQ ID NO: 134 - protein, 9 aa, synthetic construct
RPAARGSRV

SEQ ID NO: 135 - protein, 9 aa, synthetic construct
FPKKIQMLA

SEQ ID NO: 136 - protein, 10 aa, synthetic construct
FLDREQRESY

SEQ ID NO: 137 - protein, 9 aa, synthetic construct
FPAAAFPTA

SEQ ID NO: 138 - protein, 9 aa, synthetic construct
SPVTFPAAA

SEQ ID NO: 139 - protein, 10 aa, synthetic construct
FPAAAFPTAS

SEQ ID NO: 140 - protein, 9 aa, synthetic construct
ITDAHELGV

SEQ ID NO: 141 - protein, 10 aa, synthetic construct
ITDAHELGVA

SEQ ID NO: 142 - protein, 9 aa, synthetic construct
YTWPSGNIY

SEQ ID NO: 143 - protein, 10 aa, synthetic construct
VLSSLVLVPL

SEQ ID NO: 144 - protein, 9 aa, synthetic construct
NVLSSLVLV

SEQ ID NO: 145 - protein, 10 aa, synthetic construct
SLPSNVLSSL

SEQ ID NO: 146 - protein, 10 aa, synthetic construct
KIIAYQPYGK

SEQ ID NO: 147 - protein, 10 aa, synthetic construct
RLMLRKVALK

SEQ ID NO: 148 - protein, 10 aa, synthetic construct
QRLMLRKVAL

SEQ ID NO: 149 - protein, 9 aa, synthetic construct
RLMLRKVAL

SEQ ID NO: 150 - protein, 10 aa, synthetic construct
SLQRLMLRKV

SEQ ID NO: 151 - protein, 10 aa, synthetic construct
ALQSQSISLV

SEQ ID NO: 152 - protein, 9 aa, synthetic construct
ALQSQSISL

SEQ ID NO: 153 - protein, 9 aa, synthetic construct
YLLFQNTDL

SEQ ID NO: 154 - protein, 10 aa, synthetic construct
ALSPDGSIRK

SEQ ID NO: 155 - protein, 9 aa, synthetic construct
FLLTDYALS

SEQ ID NO: 156 - protein, 10 aa, synthetic construct
LLFAPEYGPK

SEQ ID NO: 157 - protein, 10 aa, synthetic construct
WRNILLLSLH

SEQ ID NO: 158 - protein, 10 aa, synthetic construct
SLHKGSLYPR

SEQ ID NO: 159 - protein, 9 aa, synthetic construct
TLLSQVNKV

SEQ ID NO: 160 - protein, 10 aa, synthetic construct
VRTLLSQVNK

SEQ ID NO: 161 - protein, 10 aa, synthetic construct
RTAPRPGSQK

SEQ ID NO: 162 - protein, 10 aa, synthetic construct
SLLRAAFFGK

SEQ ID NO: 163 - protein, 10 aa, synthetic construct
LRAAFFGKCF

SEQ ID NO: 164 - protein, 10 aa, synthetic construct
LRFNLIANQH

SEQ ID NO: 165 - protein, 9 aa, synthetic construct
KLNFRLFVI

SEQ ID NO: 166 - protein, 10 aa, synthetic construct
RKLNFRLFVI

SEQ ID NO: 167 - protein, 10 aa, synthetic construct
KLNFRLFVIR

SEQ ID NO: 168 - protein, 10 aa, synthetic construct
RIYTGEKPFK

SEQ ID NO: 169 - protein, 9 aa, synthetic construct
FEAEFTQVA

SEQ ID NO: 170 - protein, 9 aa, synthetic construct
ILMHGLVSL

SEQ ID NO: 171 - protein, 10 aa, synthetic construct
RRNDDKSILM

SEQ ID NO: 172 - protein, 10 aa, synthetic construct
SILMHGLVSL

SEQ ID NO: 173 - protein, 10 aa, synthetic construct
RRWSALVIGL

SEQ ID NO: 174 - protein, 9 aa, synthetic construct
KRRWSALVI

SEQ ID NO: 175 - protein, 9 aa, synthetic construct
RRWSALVIG

SEQ ID NO: 176 - protein, 10 aa, synthetic construct
STTKRRWSAL

SEQ ID NO: 177 - protein, 9 aa, synthetic construct
YMASEVVEV

SEQ ID NO: 178 - protein, 10 aa, synthetic construct
YMASEVVEVF

SEQ ID NO: 179 - protein, 9 aa, synthetic construct
DEQIESMTY

SEQ ID NO: 180 - protein, 10 aa, synthetic construct
KQAKVVNPPI

SEQ ID NO: 181 - protein, 9 aa, synthetic construct
FMMPRIVDV

SEQ ID NO: 182 - protein, 10 aa, synthetic construct
FMMPRIVDVT

SEQ ID NO: 183 - protein, 10 aa, synthetic construct
MTFSSTKDYV

SEQ ID NO: 184 - protein, 9 aa, synthetic construct
NEVSEVTVF

SEQ ID NO: 185 - protein, 10 aa, synthetic construct
SQFARVPGYV

SEQ ID NO: 186 - protein, 9 aa, synthetic construct
YVGSPLAAM

SEQ ID NO: 187 - protein, 9 aa, synthetic construct
SQFARVPGY

SEQ ID NO: 188 - protein, 9 aa, synthetic construct
WLVDLLPST

SEQ ID NO: 189 - protein, 9 aa, synthetic construct
EEFWLVDLL

SEQ ID NO: 190 - protein, 10 aa, synthetic construct
EEFWLVDLLP

SEQ ID NO: 191 - protein, 9 aa, synthetic construct
HLYAYHEEL

SEQ ID NO: 192 - protein, 9 aa, synthetic construct
EELSATVPS

SEQ ID NO: 193 - protein, 10 aa, synthetic construct
EELSATVPSQ

SEQ ID NO: 194 - protein, 9 aa, synthetic construct
FVADWAGTF

SEQ ID NO: 195 - protein, 10 aa, synthetic construct
EESGGAVAFF

SEQ ID NO: 196 - protein, 9 aa, synthetic construct
ESGGAVAFF

SEQ ID NO: 197 - protein, 9 aa, synthetic construct
IPLSDNTIF

SEQ ID NO: 198 - protein, 9 aa, synthetic construct
REFDKIELA

SEQ ID NO: 199 - protein, 10 aa, synthetic construct
REFDKIELAY

SEQ ID NO: 200 - protein, 10 aa, synthetic construct
SYQRYSHPLF

SEQ ID NO: 201 - protein, 10 aa, synthetic construct
IYLHGSTDKL

SEQ ID NO: 202 - protein, 9 aa, synthetic construct
YPVIFKSIM

SEQ ID NO: 203 - protein, 10 aa, synthetic construct
KEYPVIFKSI

SEQ ID NO: 204 - protein, 9 aa, synthetic construct
EYPVIFKSI

SEQ ID NO: 205 - protein, 10 aa, synthetic construct
IFKSIMRQRL

SEQ ID NO: 206 - protein, 10 aa, synthetic construct
YDYVSALHPV

SEQ ID NO: 207 - protein, 10 aa, synthetic construct
HTHYDYVSAL

SEQ ID NO: 208 - protein, 9 aa, synthetic construct
DYVSALHPV

SEQ ID NO: 209 - protein, 9 aa, synthetic construct
FPQGLPNEY

SEQ ID NO: 210 - protein, 10 aa, synthetic construct
LPNEYAFVTT

SEQ ID NO: 211 - protein, 9 aa, synthetic construct
LPNEYAFVT

SEQ ID NO: 212 - protein, 9 aa, synthetic construct
NEYAFVTTF

SEQ ID NO: 213 - protein, 9 aa, synthetic construct
QGLPNEYAF

SEQ ID NO: 214 - protein, 9 aa, synthetic construct
ALPQSILLF

SEQ ID NO: 215 - protein, 10 aa, synthetic construct
TALPQSILLF

SEQ ID NO: 216 - protein, 9 aa, synthetic construct
SPVLRSHSF

SEQ ID NO: 217 - protein, 9 aa, synthetic construct
SYQLYTHPL

SEQ ID NO: 218 - protein, 9 aa, synthetic construct
RFANPRDSF

SEQ ID NO: 219 - protein, 9 aa, synthetic construct
TIIDNIKEM

SEQ ID NO: 220 - protein, 10 aa, synthetic construct
LFSPGAANLF

SEQ ID NO: 221 - protein, 9 aa, synthetic construct
FSPGAANLF

SEQ ID NO: 222 - protein, 9 aa, synthetic construct
QPPALSPSY

SEQ ID NO: 223 - protein, 10 aa, synthetic construct
APSPGQPPAL

SEQ ID NO: 224 - protein, 10 aa, synthetic construct
FPSQRTSWEF

SEQ ID NO: 225 - protein, 10 aa, synthetic construct
MPFTTVSELM

SEQ ID NO: 226 - protein, 9 aa, synthetic construct
SELMKVSAM

SEQ ID NO: 227 - protein, 9 aa, synthetic construct
HQFHVHPLL

SEQ ID NO: 228 - protein, 10 aa, synthetic construct
MTITSRGTTV

SEQ ID NO: 229 - protein, 10 aa, synthetic construct
RYGIRGFSTI

SEQ ID NO: 230 - protein, 9 aa, synthetic construct
YGIRGFSTI

SEQ ID NO: 231 - protein, 9 aa, synthetic construct
MAGPKGFQY

SEQ ID NO: 232 - protein, 9 aa, synthetic construct
DMKARQKAL

SEQ ID NO: 233 - protein, 10 aa, synthetic construct
DMKARQKALV

SEQ ID NO: 234 - protein, 9 aa, synthetic construct
TRKNKKLAL

SEQ ID NO: 235 - protein, 10 aa, synthetic construct
QTRKNKKLAL

SEQ ID NO: 236 - protein, 10 aa, synthetic construct
LFRIKFKEPL

SEQ ID NO: 237 - protein, 9 aa, synthetic construct
ISDRFIGIY

SEQ ID NO: 238 - protein, 10 aa, synthetic construct
EISDRFIGIY

SEQ ID NO: 239 - protein, 9 aa, synthetic construct
EISDRFIGI

SEQ ID NO: 240 - protein, 10 aa, synthetic construct
QTSIQSPSLY

SEQ ID NO: 241 - protein, 9 aa, synthetic construct
TSIQSPSLY

SEQ ID NO: 242 - protein, 10 aa, synthetic construct
EMKRVFGFPV

SEQ ID NO: 243 - protein, 10 aa, synthetic construct
VVDFKKNLEY

SEQ ID NO: 244 - protein, 9 aa, synthetic construct
AAQARLQPV

SEQ ID NO: 245 - protein, 9 aa, synthetic construct
HLARHRHLM

SEQ ID NO: 246 - protein, 10 aa, synthetic construct
SPHLARHRHL

SEQ ID NO: 247 - protein, 9 aa, synthetic construct
LLDKFVEWY

SEQ ID NO: 248 - protein, 10 aa, synthetic construct
MPAWRTRGAI

SEQ ID NO: 249 - protein, 9 aa, synthetic construct
MPAWRTRGA

SEQ ID NO: 250 - protein, 10 aa, synthetic construct
LPVTRKNMPL

SEQ ID NO: 251 - protein, 9 aa, synthetic construct
WTNCILHEY

SEQ ID NO: 252 - protein, 10 aa, synthetic construct
HTLGAASSFM

SEQ ID NO: 253 - protein, 9 aa, synthetic construct
HTLGAASSF

SEQ ID NO: 254 - protein, 9 aa, synthetic construct
QLFARARPM

SEQ ID NO: 255 - protein, 10 aa, synthetic construct
ISSQPQVPFY

SEQ ID NO: 256 - protein, 9 aa, synthetic construct
SSQPQVPFY

SEQ ID NO: 257 - protein, 9 aa, synthetic construct
NVELRRNVL

SEQ ID NO: 258 - protein, 9 aa, synthetic construct
ESDLNSWPV

SEQ ID NO: 259 - protein, 10 aa, synthetic construct
LPSFRPPTAL

SEQ ID NO: 260 - protein, 9 aa, synthetic construct
ISIQRAQPL

SEQ ID NO: 261 - protein, 10 aa, synthetic construct
ESIKEITNFK

SEQ ID NO: 262 - protein, 9 aa, synthetic construct
SIKEITNFK

SEQ ID NO: 263 - protein, 9 aa, synthetic construct
ESIKEITNF

SEQ ID NO: 264 - protein, 25 aa, synthetic construct
DRSVLAKKLKFVTLVFRHGDRSPID

SEQ ID NO: 265 - protein, 22 aa, synthetic construct
NNSKKKWFLFQDSKKIQVEQPQ

SEQ ID NO: 266 - protein, 24 aa, synthetic construct
SPIKLVQKVASKIPFPDRITEESV

SEQ ID NO: 267 - protein, 23 aa, synthetic construct
TKRQVILLHTELERFLEYLPLRF

SEQ ID NO: 268 - protein, 22 aa, synthetic construct
SHTQTTLFHTFYELLIQKNKHK

SEQ ID NO: 269 - protein, 20 aa, synthetic construct
RLVLGKFGDLTNNFSSPHAR

SEQ ID NO: 270 - protein, 20 aa, synthetic construct
LSPREEFLRLCKKIMMRSIQ

SEQ ID NO: 271 - protein, 25 aa, synthetic construct
PSTANYNSFSSAPMPQIPVASVTPT

SEQ ID NO: 272 - protein, 20 aa, synthetic construct
LCPREEFLRLCKKIMMRSIQ

SEQ ID NO: 273 - protein, 22 aa, synthetic construct
SHNELADSGIPENSFNVSSLVE

SEQ ID NO: 274 - protein, 26 aa, synthetic construct
SGSPPLRVSVGDFSQEFSPIQEAQQD

SEQ ID NO: 275 - protein, 27 aa, synthetic construct
RPAGRTQLLWTPAAPTAMAEVGPGHTP

SEQ ID NO: 276 - protein, 21 aa, synthetic construct
RGQIKLADFRLARLYSSEESR

SEQ ID NO: 277 - protein, 30 aa, synthetic construct
DEQGREAELARSGPSAAGPVRLKPGLVPGL

SEQ ID NO: 278 - protein, 16 aa, synthetic construct
AAVRPEQRPAARGSRV

SEQ ID NO: 279 - protein, 17 aa, synthetic construct
TFPKKIQMLARDFLDEY

SEQ ID NO: 280 - protein, 24 aa, synthetic construct
PETGEIQVKTFLDREQRESYELKV

SEQ ID NO: 281 - protein, 23 aa, synthetic construct
PGGDSGELITDAHELGVAHPPGY

SEQ ID NO: 282 - protein, 23 aa, synthetic construct
EVVGGYTWPSGNIYQGYWAQGKR

SEQ ID NO: 283 - protein, 15 aa, synthetic construct
TIKNSDKNVVLEHFG

SEQ ID NO: 284 - protein, 29 aa, synthetic construct
TRNSFALVPSLQRLMLRKVALKNVDSSPS

SEQ ID NO: 285 - protein, 22 aa, synthetic construct
SSHYKFSKPALQSQSISLVQQS

SEQ ID NO: 286 - protein, 24 aa, synthetic construct
TETVNHHYLLFQNTDLGSFHDLLR

SEQ ID NO: 287 - protein, 23 aa, synthetic construct
DRASFLLTDYALSPDGSIRKATG

SEQ ID NO: 288 - protein, 27 aa, synthetic construct
ERFWRNILLLSLHKGSLYPRIPGLGKE

SEQ ID NO: 289 - protein, 24 aa, synthetic construct
RGRLPAGAVRTLLSQVNKVWDQSS

SEQ ID NO: 290 - protein, 25 aa, synthetic construct
GHEHQPDMQKSLLRAAFFGKCFLDR

SEQ ID NO: 291 - protein, 29 aa, synthetic construct
ELQYRGRELRFNLIANQHLLAPGFVSETR

SEQ ID NO: 292 - protein, 23 aa, synthetic construct
EDLDANLRKLNFRLFVIRGQPAD

SEQ ID NO: 293 - protein, 26 aa, synthetic construct
GHQKLPGKIHLFEAEFTQVAKKEPDG

SEQ ID NO: 294 - protein, 23 aa, synthetic construct
TTPSGSAEYMASEVVEVFTDQAT

SEQ ID NO: 295 - protein, 27 aa, synthetic construct
SVLREDLGQLEYKYQYAYFRMGIKHPD

SEQ ID NO: 296 - protein, 23 aa, synthetic construct
PENDDLFMMPRIVDVTSLATEGG

SEQ ID NO: 297 - protein, 26 aa, synthetic construct
TLDDIKEWLEDEGQVLNIQMRRTLHK

SEQ ID NO: 298 - protein, 25 aa, synthetic construct
GRMSPSQFARVPGYVGSPLAAMNPK

SEQ ID NO: 299 - protein, 25 aa, synthetic construct
KAHVEGDGVVEEIIRYHPFLYDRET

SEQ ID NO: 300 - protein, 19 aa, synthetic construct
DGVSEEFWLVDLLPSTHYT

SEQ ID NO: 301 - protein, 24 aa, synthetic construct
DSYHLYAYHEELSATVPSQWKKIG

SEQ ID NO: 302 - protein, 28 aa, synthetic construct
GDQYKATDFVADWAGTFKMVFTPKDGSG

SEQ ID NO: 303 - protein, 25 aa, synthetic construct
EYWKVLDGELEVAPEYPQSTARDWL

SEQ ID NO: 304 - protein, 25 aa, synthetic construct
TTTSVKKEELVLSEEDFQGITPGAQ

SEQ ID NO: 305 - protein, 23 aa, synthetic construct
SLTEESGGAVAFFPGNLSTSSSA

SEQ ID NO: 306 - protein, 24 aa, synthetic construct
KLRTIPLSDNTIFRRICTIAKHLE

SEQ ID NO: 307 - protein, 25 aa, synthetic construct
SHHTHSYQRYSHPLFLPGHRLDPPI

SEQ ID NO: 308 - protein, 26 aa, synthetic construct
DVTGPHLYSIYLHGSTDKLPYVTMGS

SEQ ID NO: 309 - protein, 25 aa, synthetic construct
ARLQSKEYPVIFKSIMRQRLISPQL

SEQ ID NO: 310 - protein, 24 aa, synthetic construct
LHTHYDYVSALHPVSTPSKEYTSA

SEQ ID NO: 311 - protein, 19 aa, synthetic construct
SDAFSGLTALPQSILLFGP

SEQ ID NO: 312 - protein, 22 aa, synthetic construct
SHQIHSYQLYTHPLLHPWDHRD

SEQ ID NO: 313 - protein, 23 aa, synthetic construct
STQHADLTIIDNIKEMNFLRRYK

SEQ ID NO: 314 - protein, 26 aa, synthetic construct
ASATEPANDSLFSPGAANLFSTYLAR

SEQ ID NO: 315 - protein, 27 aa, synthetic construct
AASAAAFPSQRTSWEFLQSLVSIKQEK

SEQ ID NO: 316 - protein, 26 aa, synthetic construct
GSVLQFMPFTTVSELMKVSAMSSPKV

SEQ ID NO: 317 - protein, 20 aa, synthetic construct
DKGHQFHVHPLLHSGDDLDP

SEQ ID NO: 318 - protein, 26 aa, synthetic construct
NQVLASRYGIRGFSTIKIFQKGESPV

SEQ ID NO: 319 - protein, 19 aa, synthetic construct
MAGPKGFQYRALYPFRRER

SEQ ID NO: 320 - protein, 22 aa, synthetic construct
VTLNDMKARQKALVRERERQLA

SEQ ID NO: 321 - protein, 24 aa, synthetic construct
SRLQTRKNKKLALSSTPSNIAPSD

SEQ ID NO: 322 - protein, 18 aa, synthetic construct
LNTGLFRIKFKEPLENLI

SEQ ID NO: 323 - protein, 24 aa, synthetic construct
SLRNNMFEISDRFIGIYKTYNITK

SEQ ID NO: 324 - protein, 22 aa, synthetic construct
WCTEMKRVFGFPVHYTDVSNMS

SEQ ID NO: 325 - protein, 23 aa, synthetic construct
VKQLERGEASVVDFKKNLEYAAT

SEQ ID NO: 326 - protein, 28 aa, synthetic construct
STEVEPKESPHLARHRHLMKTLVKSLST

SEQ ID NO: 327 - protein, 16 aa, synthetic construct
LMSNLAFADFCMRMYL

SEQ ID NO: 328 - protein, 24 aa, synthetic construct
TKLKSKAPHWTNCILHEYKNLSTS

SEQ ID NO: 329 - protein, 21 aa, synthetic construct
PAAGDFIRFRFFQLLRLERFF

SEQ ID NO: 330 - protein, 22 aa, synthetic construct
YLSHTLGAASSFMRPTVPPPQF

SEQ ID NO: 331 - protein, 21 aa, synthetic construct
ALLQNVELRRNVLVSPTPLAN

SEQ ID NO: 332 - protein, 23 aa, synthetic construct
FAKGFRESDLNSWPVAPRPLLSV

SEQ ID NO: 333 - protein, 30 aa, synthetic construct
GLTRISIQRAQPLPPCLPSFRPPTALQGLS

SEQ ID NO: 334 - protein, 24 aa, synthetic construct
TGKPEMDFVRLAQLFARARPMGLF

SEQ ID NO: 335 - protein, 21 aa, synthetic construct
DGAWPVLLDKFVEWYKDKQMS

SEQ ID NO: 336 - protein, 5 aa, synthetic construct
GSSSS

SEQ ID NO: 337 - protein, 4 aa, synthetic construct
GSSS

SEQ ID NO: 338 - protein, 5 aa, synthetic construct
GPGPG

SEQ ID NO: 339 - protein, 5 aa, synthetic construct
EAAAK

SEQ ID NO: 340 - protein, 12 aa, synthetic construct
HEYGAEALERAG

SEQ ID NO: 341 - protein, 4 aa, synthetic construct
RVRR

SEQ ID NO: 342 - protein, 6 aa, synthetic construct
HHHHHH

(19) United States
(12) Patent Application Publication
(10) Pub. No.: US 2025/0134976 A1
(43) Pub. Date: May 1, 2025

(54) COMPOSITIONS FOR INDUCING INTRATUMORAL IMMUNE TRIADS AND USES THEREOF

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventor: Andrea Schietinger, New York, NY (US)

(21) Appl. No.: 18/920,742

(22) Filed: Oct. 18, 2024

Related U.S. Application Data
(60) Provisional application No. 63/591,972, filed on Oct. 20, 2023.

(51) Int. Cl. A61K 39/00; A61K 39/12; A61K 45/06; A61K 47/64; A61P 35/00; C12N 7/00

(52) U.S. Cl. CPC A61K 39/001111; A61K 39/12; A61K 45/06; A61K 47/646; A61P 35/00; C12N 7/00; A61K 2039/70; C12N 2760/10034

(57) ABSTRACT

The present disclosure provides compositions for inducing intratumoral CD4 T cell::CD8 T cell::APC triads and methods for using the same to enhance the efficacy of immune checkpoint blockade (ICB) therapy, vaccines, and/or adoptive T cell transfer (ACT) to treat cancer in a subject in need thereof.

### CROSS-REFERENCE TO RELATED APPLICATIONS

This application claims the benefit of and priority to U.S. Provisional Appl. No. 63/591,972 filed Oct. 20, 2023, the contents of which are incorporated herein by reference in its entirety.

### GOVERNMENT SUPPORT

This invention was made with government support under CA269733 and CA225212 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 6, 2025, is named 115872-2818_SL.xml and is 299,772 bytes in size.

### TECHNICAL FIELD

The present disclosure relates to compositions for inducing intratumoral CD4 T cell::CD8 T cell::APC triads and methods for using the same to enhance the efficacy of immune checkpoint blockade (ICB) therapy, vaccines, and/or adoptive T cell transfer (ACT) to treat cancer in a subject in need thereof.

### BACKGROUND

The following description of the background of the present technology is provided simply as an aid in understanding the present technology and is not admitted to describe or constitute prior art to the present technology.

Tumor-reactive CD8 T cells found in cancer patients are frequently dysfunctional, unable to halt tumor growth [1]. Tumor-infiltrating dysfunctional CD8 T cells (also referred to as ‘exhausted’ T cells) commonly express high levels of inhibitory receptors (PD1, LAG3, CTLA4, TIM3) and fail to produce effector cytokines (interferon-7 (IFN-7), tumor necrosis factor-a (TNF-a)) and cytotoxic molecules (granzymes, perforin). These hallmarks of CD8 T cell dysfunction/exhaustion have been attributed to chronic tumor antigen encounter/TCR signaling and immunosuppressive signals within the tumor microenvironment [1-3].

Adoptive T cell transfer (ACT), the administration of large numbers of in vitro-generated cytolytic tumor-reactive CD8 T cells, is an important cancer immune therapy being pursued. However, a limitation of ACT is that transferred CD8 T cells often rapidly lose effector function, and despite exciting results in certain malignancies (e.g. leukemia, lymphoma), most patients still fail to achieve long-term responses, especially those with solid tumors. Factors which mitigate the efficacy of adoptively transferred CD8 T cells include poor in vivo persistence, poor tumor localization/infiltration, and rapid loss of effector function [13, 15, 16] and the loss of effector function of CD8 T cells remains a major roadblock [22, 23].

Thus, there is an urgent need for immunotherapeutic interventions that prevent or reverse CD8 T cell dysfunction/exhaustion.

### SUMMARY OF THE PRESENT TECHNOLOGY

In one aspect, the present disclosure provides a vaccine including a polypeptide comprising (i) an oligomer of at least one tumor-specific peptide epitope recognized by CD8 T cells and (ii) an oligomer of at least one tumor-specific peptide epitope recognized by CD4 T cells, wherein the oligomer of the at least one tumor-specific peptide epitope recognized by CD8 T cells is linked to the oligomer of the at least one tumor-specific peptide epitope recognized by CD4 T cells via a linker, and wherein the oligomer of the at least one tumor-specific peptide epitope recognized by CD4 T cells is at least a dimer. In another aspect, the present disclosure provides a vaccine including a nucleic acid molecule encoding a polypeptide comprising (i) an oligomer of at least one tumor-specific peptide epitope recognized by CD8 T cells and (ii) an oligomer of at least one tumor-specific peptide epitope recognized by CD4 T cells, wherein the oligomer of the at least one tumor-specific peptide epitope recognized by CD8 T cells is linked to the oligomer of the at least one tumor-specific peptide epitope recognized by CD4 T cells via a linker, and wherein the oligomer of the at least one tumor-specific peptide epitope recognized by CD4 T cells is at least a dimer. The oligomer of the at least one tumor-specific peptide epitope recognized by CD8 T cells and/or the oligomer of the at least one tumor-specific peptide epitope recognized by CD4 T cells may be a dimer, a trimer, a tetramer, a pentamer, a hexamer, a heptamer, an octamer, a nonamer, a decamer, an 11-mer, a 12-mer, a 13-mer, a 14-mer or a 15-mer. Additionally or alternatively, in some embodiments, the oligomer of the at least one tumor-specific peptide epitope recognized by CD8 T cells comprises a single tumor-specific peptide epitope. In other embodiments, the oligomer of the at least one tumor-specific peptide epitope recognized by CD8 T cells comprises 2-15 distinct tumor-specific peptide epitopes. Additionally or alternatively, in certain embodiments, the oligomer of the at least one tumor-specific peptide epitope recognized by CD4 T cells comprises a single tumor-specific peptide epitope. In other embodiments, the oligomer of the at least one tumor-specific peptide epitope recognized by CD4 T cells comprises 2-15 distinct tumor-specific peptide epitopes.

In one aspect, the present disclosure provides a vaccine comprising at least two tandem repeats of a polypeptide, wherein the polypeptide comprises (i) at least one tumor-specific peptide epitope recognized by CD8 T cells, and (ii) at least one tumor-specific peptide epitope recognized by CD4 T cells, wherein the at least one tumor-specific peptide epitope recognized by CD8 T cells is linked to the at least one tumor-specific peptide epitope recognized by CD4 T cells via a linker. Also disclosed herein is a vaccine including a nucleic acid molecule encoding at least two tandem repeats of a polypeptide, wherein the polypeptide comprises (i) at least one tumor-specific peptide epitope recognized by CD8 T cells, and (ii) at least one tumor-specific peptide epitope recognized by CD4 T cells, wherein the at least one tumor-specific peptide epitope recognized by CD8 T cells is linked to the at least one tumor-specific peptide epitope recognized by CD4 T cells via a linker. In some embodiments, the vaccine comprises at least 2, at least 3, at least 4, at least 5, at least 6, at least 7, at least 8, at least 9, at least 10, at least 11, at least 12, at least 13, at least 14, or at least 15 tandem repeats of the polypeptide. In some embodiments, the at least one tumor-specific peptide epitope recognized by CD4 T cells and/or the at least one tumor-specific peptide epitope recognized by CD8 T cells is a monomer or an oligomer. Additionally or alternatively, in certain embodiments of the vaccine, each tandem repeat of the polypeptide comprises the same tumor-specific peptide epitope recognized by CD8 T cells and/or the same tumor-specific peptide epitope recognized by CD4 T cells. In other embodiments, at least one of the at least two tandem repeats of the polypeptide comprise distinct tumor-specific peptide epitopes recognized by CD8 T cells and/or distinct tumor-specific peptide epitopes recognized by CD4 T cells.

In any of the preceding embodiments of the vaccine disclosed herein, the nucleic acid molecule comprises RNA or DNA. Additionally or alternatively, in some embodiments of the vaccines disclosed herein, the linker is a cleavable peptide linker or a rigid peptide linker. Examples of suitable linkers include, but are not limited to a GS4 linker (SEQ ID NO: 336), a GS3 linker (SEQ ID NO: 337), a P2A linker, a T2A linker, an E2A linker, a F2A linker, a BmCPV2A linker, an AAY linker, a GPGPG linker (SEQ ID NO: 338), an EAAAK linker (SEQ ID NO: 339), a HEYGAEALERAG linker (SEQ ID NO: 340), a KK linker, or an RVRR linker (SEQ ID NO: 341). In certain embodiments, the linker comprises an ubiquitination sequence. Other linkers, spacers and flanking sequences that are known in the art to enhance epitope presentation and immunogenicity may also be included in the vaccines of the present technology.

In any of the preceding embodiments of the vaccine disclosed herein, the at least one tumor-specific peptide epitope recognized by CD8 T cells is selected from among: AEPINIQTW (SEQ ID NO: 7), FPSDSWCYF (SEQ ID NO: 8), SYLDSGIIF (SEQ ID NO: 9), ACDPHSGHFV (SEQ ID NO: 10), AVCPWTWLRG (SEQ ID NO: 11), ILDKVLVHL (SEQ ID NO: 12), TLDWLLQTPK (SEQ ID NO: 13), RPHVPESAF (SEQ ID NO: 14), KIFSEVTLK (SEQ ID NO: 15), SHETVIIEL (SEQ ID NO: 16), KILDAVVAQK (SEQ ID NO: 17), FLEGNEVGKTY (SEQ ID NO: 18), EEKLIVVLF (SEQ ID NO: 19), SELFRSGLDSY (SEQ ID NO: 20), FRSGLDSYV (SEQ ID NO: 21), EAFIQPITR (SEQ ID NO: 22), KINKNPKYK (SEQ ID NO: 23), ILDTAGREEY (SEQ ID NO: 24), KELEGILLL (SEQ ID NO: 25), ETVSEQSNV (SEQ ID NO: 26), QQITKTEV (SEQ ID NO: 27), FIASNGVKLV (SEQ ID NO: 28), FLDEFMEGV (SEQ ID NO: 29), SLFEGIDIYT (SEQ ID NO: 30), VESEDIAEL (SEQ ID NO: 31), RTKVVQTLW (SEQ ID NO: 32), IPIDGIFFT (SEQ ID NO: 33), KMIGNHLWV (SEQ ID NO: 34), AMFWSVPTV (SEQ ID NO: 35), CLNEYHLFL (SEQ ID NO: 36), KLMNIQQKL (SEQ ID NO: 37), QLSCISTYV (SEQ ID NO: 38), FLYNLLTRV (SEQ ID NO: 39), IILVAVPHV (SEQ ID NO: 40), HLYASLSRA (SEQ ID NO: 41), MLGEQLFPL (SEQ ID NO: 42), RLFPGLTIKI (SEQ ID NO: 43), TRSSGSHFVF (SEQ ID NO: 44), LRTKVYAEL (SEQ ID NO: 45), LLYQELLPL (SEQ ID NO: 46), IYKAPCENW (SEQ ID NO: 47), YYPPSQIAQL (SEQ ID NO: 48), LYNGMEHLI (SEQ ID NO: 49), HSVSSAFKK (SEQ ID NO: 50), YPPPPPALL (SEQ ID NO: 51), KVDPIGHVY (SEQ ID NO: 52), LMKVDPIGHVY (SEQ ID NO: 53), KVDPIGHVYF (SEQ ID NO: 54), FVVPYMIYLL (SEQ ID NO: 55), VSVQIISCQY (SEQ ID NO: 56), VQIISCQY (SEQ ID NO: 57), CVRVSGQGL (SEQ ID NO: 58), APARLERRHSA (SEQ ID NO: 59), AYHSIEWAI (SEQ ID NO: 60), YHSIEWAI (SEQ ID NO: 61), NAYHSIEWAI (SEQ ID NO: 62), KSQREFVRR (SEQ ID NO: 63), MRMNQGVCC (SEQ ID NO: 64), FLSDHLYLV (SEQ ID NO: 65), KPSDTPRPVM (SEQ ID NO: 66), HIAKSLFEV (SEQ ID NO: 67), AGQHIAKSLF (SEQ ID NO: 68), KPFCVLISL (SEQ ID NO: 69), RPHHDQRSL (SEQ ID NO: 70), SSYTGFANK (SEQ ID NO: 71), FLLDEAIGL (SEQ ID NO: 72), ALDPHSGHFV (SEQ ID NO: 73), HSCVMASLR (SEQ ID NO: 74), HDLGRLHSC (SEQ ID NO: 75), VSKILPSTW (SEQ ID NO: 76), GVADVLLYR (SEQ ID NO: 77), TESPFEQHI (SEQ ID NO: 78), GLEREGFTF (SEQ ID NO: 79), YTDFHCQYV (SEQ ID NO: 80), CILGKLFTK (SEQ ID NO: 81), GLFGDIYLA (SEQ ID NO: 82), SLADEAEVYL (SEQ ID NO: 83), KTLTSVFQK (SEQ ID NO: 84), ILNAMIAKIJ (SEQ ID NO: 85), FAFQEYDSF (SEQ ID NO: 86), LLDIVAPK (SEQ ID NO: 87), SPMIVGSPW (SEQ ID NO: 88), ASNASSAAK (SEQ ID NO: 89), CYMEAVAL (SEQ ID NO: 90), SIY, SVGDFSQEF (SEQ ID NO: 100), VSVGDFSQEF (SEQ ID NO: 101), KLKFVTLVF (SEQ ID NO: 102), VLAKKLKFV (SEQ ID NO: 103), FLFQDSKKI (SEQ ID NO: 104), NSKKKWFLF (SEQ ID NO: 105), VQKVASKIPF (SEQ ID NO: 106), ALFASRPRF (SEQ ID NO: 107), RFLEYLPLRF (SEQ ID NO: 108), TELERFLEY (SEQ ID NO: 109), LLHTELERF (SEQ ID NO: 110), LLHTELERFL (SEQ ID NO: 111), TLFHTFYEL (SEQ ID NO: 112), TLFHTFYELL (SEQ ID NO: 113), LFHTFYELLI (SEQ ID NO: 114), LFHTFYELL (SEQ ID NO: 115), TTLFHTFYEL (SEQ ID NO: 116), KFGDLTNNF (SEQ ID NO: 117), KLFESKAEL (SEQ ID NO: 118), KLFESKAELA (SEQ ID NO: 119), YNSFSSAPM (SEQ ID NO: 120), SFSSAPMPQI (SEQ ID NO: 121), GIPENSFNV (SEQ ID NO: 122), SVGDFSQEF (SEQ ID NO: 123), VSVGDFSQEF (SEQ ID NO: 124), TPAAPTAMA (SEQ ID NO: 125), FPGNQWNPV (SEQ ID NO: 126), LADFRLARLY (SEQ ID NO: 127), NHDETSFLL (SEQ ID NO: 128), TPAHPSQGA (SEQ ID NO: 129), TPAHPSQGAV (SEQ ID NO: 130), VTEKLQPTY (SEQ ID NO: 131), HPAPPAPPPA (SEQ ID NO: 132), VPKEHPAPPA (SEQ ID NO: 133), RPAARGSRV (SEQ ID NO: 134), FPKKIQMLA (SEQ ID NO: 135), FLDREQRESY (SEQ ID NO: 136), FPAAAFPTA (SEQ ID NO: 137), SPVTFPAAA (SEQ ID NO: 138), FPAAAFPTAS (SEQ ID NO: 139), ITDAHELGV (SEQ ID NO: 140), ITDAHELGVA (SEQ ID NO: 141), YTWPSGNIY (SEQ ID NO: 142), VLSSLVLVPL (SEQ ID NO: 143), NVLSSLVLV (SEQ ID NO: 144), SLPSNVLSSL (SEQ ID NO: 145), KIIAYQPYGK (SEQ ID NO: 146), RLMLRKVALK (SEQ ID NO: 147), QRLMLRKVAL (SEQ ID NO: 148), RLMLRKVAL (SEQ ID NO: 149), SLQRLMLRKV (SEQ ID NO: 150), ALQSQSISLV (SEQ ID NO: 151), ALQSQSISL (SEQ ID NO: 152), YLLFQNTDL (SEQ ID NO: 153), ALSPDGSIRK (SEQ ID NO: 154), FLLTDYALS (SEQ ID NO: 155), LLFAPEYGPK (SEQ ID NO: 156), WRNILLLSLH (SEQ ID NO: 157), SLHKGSLYPR (SEQ ID NO: 158), TLLSQVNKV (SEQ ID NO: 159), VRTLLSQVNK (SEQ ID NO: 160), RTAPRPGSQK (SEQ ID NO: 161), SLLRAAFFGK (SEQ ID NO: 162), LRAAFFGKCF (SEQ ID NO: 163), LRFNLIANQH (SEQ ID NO: 164), KLNFRLFVI (SEQ ID NO: 165), RKLNFRLFVI (SEQ ID NO: 166), KLNFRLFVIR (SEQ ID NO: 167), RIYTGEKPFK (SEQ ID NO: 168), FEAEFTQVA (SEQ ID NO: 169), ILMHGLVSL (SEQ ID NO: 170), RRNDDKSILM (SEQ ID NO: 171), SILMHGLVSL (SEQ ID NO: 172), RRWSALVIGL (SEQ ID NO: 173), KRRWSALVI (SEQ ID NO: 174), RRWSALVIG (SEQ ID NO: 175), STTKRRWSAL (SEQ ID NO: 176), YMASEVVEV (SEQ ID NO: 177), YMASEVVEVF (SEQ ID NO: 178), DEQIESMTY (SEQ ID NO: 179), KQAKVVNPPI (SEQ ID NO: 180), FMMPRIVDV (SEQ ID NO: 181), FMMPRIVDVT (SEQ ID NO: 182), MTFSSTKDYV (SEQ ID NO: 183), NEVSEVTVF (SEQ ID NO: 184), SQFARVPGYV (SEQ ID NO: 185), YVGSPLAAM (SEQ ID NO: 186), SQFARVPGY (SEQ ID NO: 187), WLVDLLPST (SEQ ID NO: 188), EEFWLVDLL (SEQ ID NO: 189), EEFWLVDLLP (SEQ ID NO: 190), HLYAYHEEL (SEQ ID NO: 191), EELSATVPS (SEQ ID NO: 192), EELSATVPSQ (SEQ ID NO: 193), FVADWAGTF (SEQ ID NO: 194), EESGGAVAFF (SEQ ID NO: 195), ESGGAVAFF (SEQ ID NO: 196), IPLSDNTIF (SEQ ID NO: 197), REFDKIELA (SEQ ID NO: 198), REFDKIELAY (SEQ ID NO: 199), SYQRYSHPLF (SEQ ID NO: 200), IYLHGSTDKL (SEQ ID NO: 201), YPVIFKSIM (SEQ ID NO: 202), KEYPVIFKSI (SEQ ID NO: 203), EYPVIFKSI (SEQ ID NO: 204), IFKSIMRQRL (SEQ ID NO: 205), YDYVSALHPV (SEQ ID NO: 206), HTHYDYVSAL (SEQ ID NO: 207), DYVSALHPV (SEQ ID NO: 208), FPQGLPNEY (SEQ ID NO: 209), LPNEYAFVTT (SEQ ID NO: 210), LPNEYAFVT (SEQ ID NO: 211), NEYAFVTTF (SEQ ID NO: 212), QGLPNEYAF (SEQ ID NO: 213), ALPQSILLF (SEQ ID NO: 214), TALPQSILLF (SEQ ID NO: 215), SPVLRSHSF (SEQ ID NO: 216), SYQLYTHPL (SEQ ID NO: 217), RFANPRDSF (SEQ ID NO: 218), TIIDNIKEM (SEQ ID NO: 219), LFSPGAANLF (SEQ ID NO: 220), FSPGAANLF (SEQ ID NO: 221), QPPALSPSY (SEQ ID NO: 222), APSPGQPPAL (SEQ ID NO: 223), FPSQRTSWEF (SEQ ID NO: 224), MPFTTVSELM (SEQ ID NO: 225), SELMKVSAM (SEQ ID NO: 226), HQFHVHPLL (SEQ ID NO: 227), MTITSRGTTV (SEQ ID NO: 228), RYGIRGFSTI (SEQ ID NO: 229), YGIRGFSTI (SEQ ID NO: 230), MAGPKGFQY (SEQ ID NO: 231), DMKARQKAL (SEQ ID NO: 232), DMKARQKALV (SEQ ID NO: 233), TRKNKKLAL (SEQ ID NO: 234), QTRKNKKLAL (SEQ ID NO: 235), LFRIKFKEPL (SEQ ID NO: 236), ISDRFIGIY (SEQ ID NO: 237), EISDRFIGIY (SEQ ID NO: 238), EISDRFIGI (SEQ ID NO: 239), QTSIQSPSLY (SEQ ID NO: 240), TSIQSPSLY (SEQ ID NO: 241), EMKRVFGFPV (SEQ ID NO: 242), VVDFKKNLEY (SEQ ID NO: 243), AAQARLQPV (SEQ ID NO: 244), HLARHRHLM (SEQ ID NO: 245), SPHLARHRHL (SEQ ID NO: 246), LLDKFVEWY (SEQ ID NO: 247), MPAWRTRGAI (SEQ ID NO: 248), MPAWRTRGA (SEQ ID NO: 249), LPVTRKNMPL (SEQ ID NO: 250), WTNCILTHEY (SEQ ID NO: 251), HTLGAASSFM (SEQ ID NO: 252), HTLGAASSF (SEQ ID NO: 253), QLFARARPM (SEQ ID NO: 254), ISSQPQVPFY (SEQ ID NO: 255), SSQPQVPFY (SEQ ID NO: 256), NVELRRNVL (SEQ ID NO: 257), ESDLNSWPV (SEQ ID NO: 258), LPSFRPPTAL (SEQ ID NO: 259), ISIQRAQPL (SEQ ID NO: 260), ESIKEITNFK (SEQ ID NO: 261), SIKEITNFK (SEQ ID NO: 262), and ESIKEITNF (SEQ ID NO: 263).

In any of the foregoing embodiments of the vaccine disclosed herein, the at least one tumor-specific peptide epitope recognized by CD4 T cells is selected from among:

(SEQ ID NO: 91)
TLYQDDTLTLQAAGE,
(SEQ ID NO: 92)
YSVYFNLPADTIYTNH,
(SEQ ID NO: 93)
FSWAMDLDPKGAE
(SEQ ID NO: 94)
MIFEKHGFRRTTPP,
(SEQ ID NO: 95)
WRRAPAPGA,
(SEQ ID NO: 96)
PVTWRRAPA,
(SEQ ID NO: 97)
RVIKNSIRLTLE,
(SEQ ID NO: 98)
PYYFAAELPPRNLPEP,
(SEQ ID NO: 99)
GELIGILNAAKVPAD,
HEL,
(SEQ ID NO: 264)
DRSVLAKKLKFVTLVFRHGDRSPID,
(SEQ ID NO: 265)
NNSKKKWFLFQDSKKIQVEQPQ,
(SEQ ID NO: 266)
SPIKLVQKVASKIPFPDRITEESV,
(SEQ ID NO: 267)
TKRQVILLHTELERFLEYLPLRF,
(SEQ ID NO: 268)
SHTQTTLFHTFYELLIQKNKHK,
(SEQ ID NO: 269)
RLVLGKFGDLINNFSSPHAR,
(SEQ ID NO: 270)
LSPREEFLRLCKKIMMRSIQ,
(SEQ ID NO: 271)
PSTANYNSFSSAPMPQIPVASVTPT,
(SEQ ID NO: 272)
LCPREEFLRLCKKIMMRSIQ,
(SEQ ID NO: 273)
SHNELADSGIPENSFNVSSLVE,
(SEQ ID NO: 274)
SGSPPLRVSVGDFSQEFSPIQEAQQD,
(SEQ ID NO: 275)
RPAGRTQLLWTPAAPTAMAEVGPGHTP,
(SEQ ID NO: 276)
RGQIKLADFRLARLYSSEESR,
(SEQ ID NO: 277)
DEQGREAELARSGPSAAGPVRLKPGLVPGL,
(SEQ ID NO: 278)
AAVRPEQRPAARGSRV,
(SEQ ID NO: 279)
TFPKKIQMLARDFLDEY,
(SEQ ID NO: 280)
PETGEIQVKTFLDREQRESYELKV,
(SEQ ID NO: 281)
PGGDSGELITDAHELGVAHPPGY,
(SEQ ID NO: 282)
EVVGGYTWPSGNIYQGYWAQGKR,
(SEQ ID NO: 283)
TIKNSDKNVVLEHFG,
(SEQ ID NO: 284)
TRNSFALVPSLQRLMLRKVALKNVDSSPS,
(SEQ ID NO: 285)
SSHYKFSKPALQSQSISLVQQS,
(SEQ ID NO: 286)
TETVNHHYLLFQNTDLGSFHDLLR,
(SEQ ID NO: 287)
DRASFLLTDYALSPDGSIRKATG,
(SEQ ID NO: 288)
ERFWRNILLLSLHKGSLYPRIPGLGKE,
(SEQ ID NO: 289)
RGRLPAGAVRTLLSQVNKVWDQSS,
(SEQ ID NO: 290)
GHEHQPDMQKSLLRAAFFGKCFLDR,
(SEQ ID NO: 291)
ELQYRGRELRFNLIANQHLLAPGFVSETR,
(SEQ ID NO: 292)
EDLDANLRKLNFRLFVIRGQPAD,
(SEQ ID NO: 293)
GHQKLPGKIHLFEAEFTQVAKKEPDG,
(SEQ ID NO: 294)
TTPSGSAEYMASEVVEVFTDQAT,
(SEQ ID NO: 295)
SVLREDLGQLEYKYQYAYFRMGIKHPD,
(SEQ ID NO: 296)
PENDDLFMMPRIVDVTSLATEGG,
(SEQ ID NO: 297)
TLDDIKEWLEDEGQVLNIQMRRTLHK,
(SEQ ID NO: 298)
GRMSPSQFARVPGYVGSPLAAMNPK,
(SEQ ID NO: 299)
KAHVEGDGVVEEIIRYHPFLYDRET,
(SEQ ID NO: 300)
DGVSEEFWLVDLLPSTHYT,
(SEQ ID NO: 301)
DSYHLYAYHEELSATVPSQWKKIG,
(SEQ ID NO: 302)
GDQYKATDFVADWAGTFKMVFTPKDGSG,
(SEQ ID NO: 303)
EYWKVLDGELEVAPEYPQSTARDWL,
(SEQ ID NO: 304)
TTTSVKKEELVLSEEDFQGITPGAQ,
(SEQ ID NO: 305)
SLTEESGGAVAFFPGNLSTSSSA,
(SEQ ID NO: 306)
KLRTIPLSDNTIFRRICTIAKHLE,
(SEQ ID NO: 307)
SHHTHSYQRYSHPLFLPGHRLDPPI,
(SEQ ID NO: 308)
DVTGPHLYSIYLHGSTDKLPYVTMGS,
(SEQ ID NO: 309)
ARLQSKEYPVIFKSIMRQRLISPQL,
(SEQ ID NO: 310)
LHTHYDYVSALHPVSTPSKEYTSA,
(SEQ ID NO: 311)
SDAFSGLTALPQSILLFGP,
(SEQ ID NO: 312)
SHQIHSYQLYTHPLLHPWDHRD,
(SEQ ID NO: 313)
STQHADLTIIDNIKEMNFLRRYK,
(SEQ ID NO: 314)
ASATEPANDSLFSPGAANLFSTYLAR,
(SEQ ID NO: 315)
AASAAAFPSQRTSWEFLQSLVSIKQEK,
(SEQ ID NO: 316)
GSVLQFMPFTTVSELMKVSAMSSPKV,
(SEQ ID NO: 317)
DKGHQFHVHPLLHSGDDLDP,
(SEQ ID NO: 318)
NQVLASRYGIRGFSTIKIFQKGESPV,
(SEQ ID NO: 319)
MAGPKGFQYRALYPFRRER,
(SEQ ID NO: 320)
VTLNDMKARQKALVRERERQLA,
(SEQ ID NO: 321)
SRLQTRKNKKLALSSTPSNIAPSD,
(SEQ ID NO: 322)
LNTGLFRIKFKEPLENLI,
(SEQ ID NO: 323)
SLRNNMFEISDRFIGIYKTYNITK,
(SEQ ID NO: 324)
WCTEMKRVFGFPVHYTDVSNMS,
(SEQ ID NO: 325)
VKQLERGEASVVDFKKNLEYAAT,
(SEQ ID NO: 326)
STEVEPKESPHLARHRHLMKTLVKSLST,
(SEQ ID NO: 327)
LMSNLAFADFCMRMYL,
(SEQ ID NO: 328)
TKLKSKAPHWTNCILHEYKNLSTS,
(SEQ ID NO: 329)
PAAGDFIRFRFFQLLRLERFF,
(SEQ ID NO: 330)
YLSHTLGAASSFMRPTVPPPQF,
(SEQ ID NO: 331)
ALLQNVELRRNVLVSPTPLAN,
(SEQ ID NO: 332)
FAKGFRESDLNSWPVAPRPLLSV,
(SEQ ID NO: 333)
GLTRISIQRAQPLPPCLPSFRPPTALQGLS,
(SEQ ID NO: 334)
TGKPEMDFVRLAQLFARARPMGLF,
and
(SEQ ID NO: 335)
DGAWPVLLDKFVEWYKDKQMS.

In any and all embodiments of the vaccines of the present technology, the at least one tumor-specific peptide epitope recognized by CD8 T cells and/or the at least one tumor-specific peptide epitope recognized by CD4 T cells is a personalized neoantigen or neoepitope specific for a cancer subject. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

In any and all embodiments of the vaccine disclosed herein, the at least one tumor-specific peptide epitope recognized by CD8 T cells and/or the at least one tumor-specific peptide epitope recognized by CD4 T cells is a tumor antigen. Examples of tumor-specific peptide epitopes that are derived from one or more tumor antigens and recognized by CD8 T cells include but are not limited to MAGE, BAGE, GAGE, NY-ESO-1, Tyrosinase, Melan-A, gp100, CEA, MART-1, HER2, WT1, MUC1, ppCT, Beta-catenin, CDK4, LPGAT1, CASP-8, CDKN2A, HLA-A11d, CLPP, GPNMB, RBAF600, SIRT2, SNRPD1, SNRP116, MART2, MUM-if, MUM-2, MUM-3, Myosin class I, N-ras, OS-9, Elongation factor 2, NFYC, Alpha-actinin-4, Malic enzyme, HLA-A2, Hsp70-2, SETDB1, METTL17, ALDH1A1, CDKN2A, TKT, SEC24A, EXOC8, MRPS5, PABPC1, KIF2C, POLA2, CCT6A, TRRAP, DNMT1, PABPC3, MAGE-A10, FMN2, TMEM48, AKAP13, OR8B3, WASL, MAGEA6, PDS5A, MED13, FLNA, KIB1B, KFI1BP, NARFL, PPFIA4, CDC37L1, MLL3, FLNA, DOPEY2, TTBK2, KIF26B, SPOP, RETSAT, CLINT1, COX7A2, FAM3C, CSMD1, PPP1R3B, CDK12, CSNK1A1, GAS7, MATN, HAUS3, MTFR2, CHTF18, MYADM, HERC1 and HSDL1. Examples of tumor-specific peptide epitopes that are derived from one or more tumor antigens and recognized by CD4 T cells include but are not limited to COA-1, ARTC1, CDC27, FN1, LDLR-FUT fusion protein, neo-PAP, PTPRK and Triosephosphate isomerase.

In one aspect, the present disclosure provides a method for treating cancer in a subject in need thereof comprising administering to the subject an effective amount of any and all embodiments of the vaccine described herein.

In another aspect, the present disclosure provides a method for treating cancer in a subject in need thereof comprising administering to the subject an effective amount of any and all embodiments of the vaccine described herein and sequentially, simultaneously or separately administering to the subject an effective amount of an immune checkpoint inhibitor. In some embodiments, the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Examples of immune checkpoint inhibitors include, but are not limited to, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

In one aspect, the present disclosure provides a method for treating cancer in a subject in need thereof comprising administering to the subject an effective amount of any and all embodiments of the vaccine described herein and sequentially, simultaneously or separately administering to the subject an effective amount of a T-cell-engaging multi-specific antibody. The T-cell-engaging multi-specific antibody may be a bispecific T cell engager (BiTE), a dual-affinity retargeting antibody (DART), a TandAb, a XmAb, a BITE-Fc, a 2:1 Crossmab, a duobody, a knobs-into-holes (KiH) antibody, or an IgG-scFv bispecific antibody. Additionally or alternatively, in some embodiments of the methods disclosed herein, the T-cell-engaging multi-specific antibody specifically targets one or more target antigens selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

In yet another aspect, the present disclosure provides a method for treating cancer in a subject in need thereof comprising administering to the subject an effective amount of any and all embodiments of the vaccine described herein and sequentially, simultaneously or separately administering to the subject an effective amount of an adoptive cell therapeutic composition (e.g., T cells), optionally wherein the adoptive cell therapeutic composition is obtained from a donor. The adoptive cell therapeutic compositions of the present technology comprise one or more of tumor infiltrating T cells, CD8+ T cells, CD4+ T cells, delta-gamma T-cells, and alpha-beta T-cells. In some embodiments, the adoptive cell therapeutic compositions provided herein express a T-cell receptor (TCR) or other cell-surface ligand that binds to a target antigen, such as a tumor antigen. In some embodiments, the T cell receptor is a wild-type or native T-cell receptor. In some embodiments, the TCR is an engineered receptor or a non-native receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR). Additionally or alternatively, in some embodiments of the methods disclosed herein, the donor and the subject are the same or different.

In any of the preceding embodiments, the methods of the present technology further comprise administering a cytokine to the subject. The cytokine may be administered prior to, during, or subsequent to administration of the adoptive cell therapeutic composition. Examples of cytokines include, but are not limited to, interferon a, interferon β, interferon γ, complement C5a, IL-2, TNF alpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCRIO, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In any and all embodiments of the methods disclosed herein, the vaccine is administered pleurally, topically, parenterally, intravenously, subcutaneously, intranodally, intratumorally, intrathecally, intrapleurally or intraperitoneally. In any of the foregoing embodiments of the methods disclosed herein, the immune checkpoint inhibitor, the T-cell-engaging multi-specific antibody or the adoptive cell therapeutic composition is administered pleurally, topically, parenterally, intravenously, subcutaneously, intranodally, intratumorally, intrathecally, intrapleurally or intraperitoneally. In some embodiments, intratumoral administration comprises direct administration to a tumor or administration in close proximity to a tumor. In other embodiments, intratumoral administration comprises delivery via an oncolytic virus or an APC vaccine.

In any and all embodiments of the methods disclosed herein, the cancer is a carcinoma, sarcoma, a solid non-hematopoietic cancer, or a hematopoietic cancer. In certain embodiments of the methods disclosed herein, the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, glioblastomas, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

### BRIEF DESCRIPTION OF THE DRAWINGS

FIGS. 1A-1I. Tumor-specific CD4 T cells prevent and reverse CD8 T cell dysfunction/exhaustion within solid tumors and mediate tumor elimination. FIG. 1A. Scheme: tumor models, adoptively transferred effector T cells, and experimental schemes. FIG. 1B. B16 OVA-GP₆₁₋₈₀ (B16-OG) tumor growth (left) and Kaplan-Meier survival curve (right) of tumor-bearing B6 WT mice (CD45.2; Thy1.2) receiving effector TCR_{OTI} CD8 T cells alone (CD45.1) (TCR_{OT1}) or together with TCR_{SMARTA} CD4 T cells (Thy1.1) (TCR_{OT1}^{(+CD4)}) as indicated (ACT=adoptive T cell transfer). Data is representative of 5 independent experiments (n=5 mice/group). Values are mean±SEM. Significance is calculated by multiple t test. Kaplan-Meier curve; **p=000021; Mantel-Cox test. FIG. 1C. MCA205 OVA-GP₆₁₋₈₀ (MCA-OG) tumor outgrowth and survival in B6 mice treated as described in FIG. 1B; **p=0.0003; Mantel-Cox test. Data is representative of 2 independent experiments (n==5-6 mice/group). FIG. 1D. TCR_{OTI} (% of total of CD8+ T cells) within progressing B16-OG tumors 8-9 days post transfer+/−TCR_{SMARTA} CD4 T cells. Data pooled from 2 independent experiments (n=8 mice/group). Each symbol represents an individual mouse. FIG. 1E. IFNγ and TNFα production of TCR_{OTI} isolated from B16-OG tumors 8-9 days post transfer+/−TCR_{SMARTA} CD4 T cells. Cytokine production was assessed after 4-hr peptide stimulation ex vivo. Data show 2 pooled independent experiments (n=5-7). FIG. 1F. Inhibitory receptor expression, and FIG. 1G. TOX expression of B16-OG tumor-infiltrating TCR_{OTI} isolated 8-9 days post transfer+/−TCR_{SMARTA}. Graphs depict relative MFI normalized to naive TCR_{OTI}; two pooled independent experiments (n=5-7 mice/group). FIG. 1H. Mice with B16-OG tumors received effector TCR_{OTI} CD8 T cells 14 days post tumor transplantation; 9 days later, TCR_{SMARTA} CD4 T cells were adoptively transferred (black triangle); B16-OG tumor growth in mice receiving only TCR_{OT1} are shown in black circles. Data is representative of 2 independent experiments (n=8 mice/group). Values are mean±SEM. Significance is calculated by multiple t test. FIG. 1I. IFNγ and TNFα production of TCR_{OTI} isolated from cohorts presented in FIG. 1h.

FIGS. 2A-2G. Tumor-specific CD4 T cells transcriptionally and epigenetically reprogram tumor-specific CD8 T cells and prevent terminal differentiation/exhaustion. FIG. 2A. MA plot of RNA-seq data showing the relationship between average expression and expression changes of TCR_{OT1} and TCR (+^{CD4}) TIL. Statistically significantly DEGs (false discovery rate (FDR)<0.05) are shown with select genes highlighted for reference. FIG. 2B. Heat map of RNA-seq expression (normalized counts after variance stabilizing transformation, centered and scaled by row for DEGs) (FDR<0.05) in TCR_{OT1} and TCR^{(+CD4)} TIL. FIG. 2C. Selected GO terms enriched for genes up-regulated in TCR_{OT1} (bottom terms) and TCR (+^{CD4}) (upper terms) TTL. FIG. 2D. Chromatin accessibility (ATAC-seq); (left) heatmap of log 2-transformed normalized read counts transformed with variance stabilization per for regions with differential chromatin accessibility; (right) each row represents one peak (differentially accessible between TCR_{OT1} and TCR^{(+CD4)} TIL; FDR<0.05) displayed over a 2-kb window centered on the peak summit; regions were clustered with k-means clustering. Genes associated with the two major clusters are highlighted. FIG. 2E. ATAC-seq signal profiles across the Tox, Pdcd1, Lag3, Tcf7, and Lef1 loci. Peaks significantly lost or gained are highlighted with square black boxes. FIG. 2F. Top 10 most-significantly enriched transcription factor motifs in peaks with increased accessibility in TCR_{OT1}^{(+CD4)} TIL (top) or TCR TIL (bottom). FIG. 2G. Enrichment of gene sets in TCR and TCR_{OT1} (+^{CD4}) respectively, described for human tumor infiltrating (TIL) CD8 T cell subsets (CD69−CD39−) stem-like CD8 T cells/TIL (responders) or (CD69+CD39+) terminally differentiated CD8 T cells/TIL (non-responders) from metastatic melanoma patients receiving ex vivo expanded TIL for ACT (S. Krishna et al, Science 2020). TCR^{(+CD4)} are enriched in genes observed in CD69-CD39-stem-like T cells/TIL from responders in contrast to TCR_{OT1} which are positively enriched for genes in CD69+ CD39+ terminally differentiated CD8 T cells/TIL from non-responders. NES, normalized enrichment score.

FIGS. 3A-3H. Tumor elimination requires tumor antigen/epitope linkage and unique spatial orientation of tumor-specific CD8 T cells, CD4 T cells and CD11c+ dendritic cells (DC) within tumors. FIG. 3A. B16-OG tumor outgrowth in CD11c-DTR/GFP bone marrow (BM) chimeras (scheme, top; DTR→WT or WT→WT) treated with diphtheria toxin (DT). In vitro activated TCR_{OTI} and TCR_{SMARTA} were adoptively transferred into lymphodepleted tumor-bearing BM chimeras. 5 days post ACT, mice were treated with DT. Representative of 2 independent experiments (n=3 mice/group). Values are mean±SEM. Significance is calculated by multiple t test. FIG. 3B. (Top) Experimental scheme of tumor models A and B: 2.5×10⁶ B16-OG cancer cells (B16 OG; model A) or 1.25×10⁶ B16-OVA (B16-0) mixed with 1.25×10⁶ B16-GP₆₁₋₈₀ cancer cells (B16 O+G; model B) were transplanted into B6 WT mice. (Bottom), (left) Tumor outgrowth of B16-OG or B16 O+G tumors after TCR_{OTI} and TCR_{SMARTA} ACT. Representative of 2 independent experiments (n=7 mice/cohort). Data are shown as mean±SEM. Significance is calculated by multiple t test. (Right) Kaplan-Meier curve; **p=0.0002; Mantel-Cox test. FIG. 3C. Percentage of TCR_{OT1} (+^{CD4}) (out of total CD8+ TIL) 9 days post ACT. FIG. 3D. Percentage of TCR_{SMARTA} (out of total CD4+ TIL) 9 days post ACT. Data represent 2 pooled, independent experiments (n=8 mice/tumor model). Each symbol represents an individual mouse. FIG. 3E. IFNγ, TNFα, CD107, Granzyme B production of TCR_{OT1}^{(+CD4)} isolated from B16-OG or B16 O+G tumors, or FIG. 3F. isolated from tumor-draining lymph nodes of B16-OG or B16 O+G tumor-bearing hosts. Cytotoxic molecules and cytokine production assessed after 4-hr peptide stimulation ex vivo. Representative of 2 independent experiments (n=3 mice/tumor). Data are shown as mean±SEM. *p<0.05, unpaired two tailed Student's t test. NS, not significant. FIG. 3G. Mosaic, clonal growth of B16 OVA-EGFP mixed with B16 GP₆₁₋₈₀-Cerulean tumor cells (B16 O+G) in B6 WT mice. Shown are confocal microscopy sections of tumors with B16 OVA (dashed lines) and B16 GP (solid lines) distinct tumor regions as shown with arrows. FIG. 3H. Proposed model: Triad formation (three-cell-type clusters; CD8 T cells::CD4 T cells:: APC) form in B16 OG tumors (Model A) where CD8- and CD4-tumor antigens/epitopes are linked and co-presented on the same APC within tumors; tumor-specific CD8 and CD4 T cells engage on same APC; CD4 T cells reprogram CD8 T cells. Model B: B16 O+G; triads cannot form due to CD8- and CD4-tumor antigens being presented on distinct APC.

FIGS. 4A-4E. Intratumoral immune triads (three-cell-types clusters; CD8 T cell::CD4 T cell::APC) are required for CD8 T cell reprogramming and tumor elimination. FIG. 4A. Color-coded mouse models to determine intratumoral immune triad formation (Models A and B (see FIG. 3)). B16 OG (Model A) or B16 O+G (Model B) tumors were established in CD11c-YFP mice; effector TCR_{OTI}-RFP, and TCR_{SMARTA}-EGFP T cells were adoptively transferred into tumor-bearing hosts. Confocal microscopy analysis of frozen tumor tissue sections. Arrows indicate triads. FIG. 4B. Numbers of triads per field of view (FOV), and FIG. 4C. (left) Fold increase of triads normalized to total numbers of CD11c⁺YFP⁺ cells/FOV (right).

FIG. 4D. Quantification of fold increase of numbers of CD4 T cell-DC dyads normalized to total number of infiltrating CD11c⁺YFP⁺ cells/FOV. Each symbol represents an individual frozen tumor section (n=3 mice/group/model). Data are shown as mean±SEM. *** P<0.001, unpaired two-tailed Student's t test. FIG. 4E. Proposed model of TRIAD-associated cancer elimination.

FIGS. 5A-5C. FIG. 5A. IFNγ and TNFα production, FIG. 5B. inhibitory receptor expression, and FIG. 5C. TOX expression of TCR_{OTI} isolated from tumor-draining lymph nodes of B16-OG tumor-bearing mice 8-9 days post transfer+/−TCR_{SMARTA}. Black circle represents mice receiving only TCR_{OTI} and white triangles represent mice which received TCR_{OTI} together with TCR_{SMARTA}. Cytokine production was assessed after 4-hr peptide stimulation ex vivo. Data show 2 pooled independent experiments (n=5-7). Data are shown as mean±SEM. *p<0.05, using unpaired two-tailed Student's t test. ns, not significant.

FIG. 6. Enrichment of gene sets in TCR_{OT1} and TCR_{OT1}^{(+CD4)}, respectively, described for human tumor infiltrating (TIL) CD8 T cells from metastatic melanoma patients receiving ex vivo expanded CD8+ TIL in adoptive T cell transfers (ACT) (S. Krishna et al, Science 2020). ACT responders contained CD69− CD39− stem-like CD8+ TIL, which were lacking in ACT-non-responders. ACT non-responders contained CD69+ CD39+ terminally differentiated CD8+ TIL. TCR_{OT1}^{(+CD4)} are enriched in genes observed in CD69− CD39− stem-like T cells/TIL and are negatively enriched for genes from CD69+ CD39+ terminally differentiated CD8 T cells/TIL. Significantly differentially expressed, enriched genes are shown. See also main FIG. 2g.

FIGS. 7A-7C. FIG. 7A. Flow cytometric analysis of MHC class II I-Ab expression on parental B16 tumor cells cultured in vitro (grey) or after isolation from tumor bearing B6 WT mice ex vivo (black). FIG. 7B. I-Ab expression on B16-OG tumor cells (parental; black) or CRISPR/Cas9 gene-edited B16 OG I-Ab-deficient cells (KO; grey) after 48 hours IFNγ treatment in vitro. FIG. 7C. Outgrowth of B16-OG I-Ab-deficient tumors in B6 WT mice receiving adoptively transferred in vitro activated TCR_{OT1} and TCR_{SMARTA} (black triangle) or TCR_{OT1} only (black circle).

FIG. 8. Triads are required for immune-checkpoint blockade induced tumor elimination. 2.5×10⁶ B16-OG cancer cells (B16 OG; black triangle) or 1.25×10⁶ B16-OVA (B16-0) mixed with 1.25×10⁶ B16-GP61-cancer cells (B16 O+G; white circle) were transplanted into B6 WT mice (n=5/cohort). At day 7 post tumor transplantation, mice were treated with anti-PD1 and anti-PDL1 mAb every other day for one week. While B16-OG tumors are rejected, B16 O+G tumors continue to grow, demonstrating the requirement of triads for tumor elimination in response to immune checkpoint blockade.

FIG. 9 shows exemplary structural configurations of the vaccines of the present technology comprising tumor-specific CD8 epitopes and tumor-specific CD4 epitopes.

FIG. 10. B16-OG tumors in P2-microglobulin-deficient and MHC-II-deficient mixed bone marrow chimeras fail T cell mediated tumor elimination demonstrating that CD8 and CD4 T cells must engage on the same APC. Mixed bone marrow (BM) chimeras were generated by transfer of 5×10⁶ congenic (CD45.1) β2m-deficient BM cells and 5×10⁶ MHC-II-deficient BM cells into lethally irradiated (CD45.2) B6 WT mice (n=6; red). As control chimeras, B6 WT mice were reconstituted with 10⁶ BM cells from CD45.1 B6 WT mice (n=6; black). After approx. 2 months post BM transplantation, mice were injected s.c. with B16 OVA-GP61 (B16-OG) tumor cells, and once established, in vitro activated TCR_{OT1} and TCR_{SMARTA} T cells were adoptively transferred. Shown are tumor growth curves. ***p<0.001; 2-way ANOVA with Tukey test

FIG. 11. Tumor-specific CD4 T cells are required for CD8 T cell-mediated tumor elimination. Outgrowth of B16-OG tumors in B6 WT mice receiving adoptively transferred in vitro activated TCR_{OT1} and TCR_{SMARTA} T cells (ACT) at indicated time points (Exp 1: day 20 post tumor transplantation; Exp 2: day 16 post tumor transplantation; black arrows), followed by CD4-depleting monoclonal antibody treatment (anti-CD4 mAb 5-6 days post ACT (Exp 1: day 25; Exp 2: day 22); dashed arrows). Tumor volume was monitored. Two independent experiments with n=3 (experiment 1) and n=4 (experiment 2) mice, respectively.

FIG. 12. Triad formation is also required for immune checkpoint blockade (anti-PD1 and anti-PDL1). Outgrowth of B16-OG (gray solid) and B16 O+G (black dash) tumors in B6 WT mice receiving anti-PD1 and anti-PDL1 blocking monoclonal antibodies (mAb) at indicated time points (days 12, 14, 16, 18, 20 and 22 post tumor transplantation; black arrows). Tumor volume was monitored. Data is representative of 2 independent experiments. (Right) Tumor volume at day 22 post tumor implantation following checkpoint blockade. Data are shown as mean±SEM. *p=0.02, using unpaired two-tailed Student's t test.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that triad cell clusters formed by tumor-specific CD8 and CD4 T cells, and antigen presenting cells (APC) are useful to overcome CD8 T cell dysfunction in the setting of ACT. As demonstrated by the Examples described herein, when CD8- and CD4-tumor antigens/epitopes are linked, they are co-presented on the same APC within tumors. Consequently, tumor-specific CD8 and CD4 T cells engage on same APC, thus inducing formation of the triad cell clusters. CD4 T cells co-engage with CD8 T cells and APC cross-presenting CD8- and CD4-tumor antigens during the priming and/or effector phase, forming a three-cell-cluster (triad), to license CD8 T cell cytotoxicity and mediate cancer cell elimination. Triad formation transcriptionally and epigenetically reprograms CD8 T cells, prevents T cell dysfunction/exhaustion, and ultimately leads to the elimination of large established tumors and confer long-term protection from recurrence. Strikingly, the formation of CD4 T cell::CD8 T cell::APC triads in tumors of patients with lung cancers treated with immune checkpoint blockade (ICB) was associated with clinical responses. In contrast, triads cannot form when CD8- and CD4-tumor antigens are presented on distinct APCs, which negatively impacts anti-tumor immunity and cancer elimination.

In order to ensure triad formation, a sufficient quantity and appropriate spacing of tumor-specific CD8 and CD4 epitopes must be presented on the surface of a single APC. The overall quantity and spacing of tumor-specific CD8 and CD4 epitopes is critical because these epitopes must compete with other endogenous (non-tumor) epitopes. To ensure that enough tumor-specific epitopes are being presented, numerous repeat-sequences of tumor-specific epitopes (e.g., at least two (dimer) tandem repeats) have to be engineered within the vaccine to increase the possibility that enough tumor-specific CD8 and CD4 epitopes are concurrently being presented on the surface of APCs to allow CD4 T cell-CD8 T cell-APC triad formation.

Taken together, the methods disclosed herein enforce the formation of CD4 T cell-CD8 T cell-APC triads, thereby enhancing the therapeutic efficacy of ICB, vaccine and ACT against solid tumors.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes and CAR (i.e. chimeric antigen receptor) modified lymphocytes (e.g., CAR T cells). In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

An “antigen” is a molecule or entity to which an antibody or a T cell receptor binds. In some embodiments, an antigen is or comprises a polypeptide or portion thereof. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen. As used herein, the term “poorly immunogenic antigen” refers to an antigen that does not elicit a protective or therapeutically effective response in a patient, e.g., an antigen that does not induce an immune response that is sufficient to treat or prevent a disease or condition described herein or one or more signs or symptoms associated with a disease or condition described herein. Antigens that are expressed in or by tumor cells are referred to as “tumor associated antigens.” A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not produce an immune response against healthy tissue or be masked by the protective components of the immune system.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein, a “cleavable peptide”, which is also referred to as a “cleavable linker,” means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. Sci Rep. 2015; 5:16273. Published 2015 Nov. 5. As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 1), wherein X refers to any amino acid generally thought to be self-cleaving.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “epitope” refers to a portion of an antigen that is recognized by the immune system in the appropriate context, specifically by antibodies, B cells, or T cells Epitopes may include B cell epitopes (e.g. predicted B cell reactive epitopes) and T cell epitopes (e.g., predicted T cell reactive epitopes). B cell epitopes (e.g., predicted B cell reactive epitopes) refer to a specific region of the antigen that is recognized by an antibody. T-cell epitopes (e.g., predicted T cell reactive epitopes) are peptide sequences which, in association with proteins on APC, are required for recognition by specific T-cells. T cell epitopes (e.g., predicted T cell reactive epitopes) are processed intracellularly and presented on the surface of APCs, where they are bound to MHC molecules including MHC class II and MHC class I molecules. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). Conformational epitopes are epitopes that are defined by the conformational structure of the native protein. These epitopes may be continuous or discontinuous (i.e., may be components of the epitope can be situated on disparate parts of the protein, which are brought close to each other in the folded native protein structure).

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

“Immune checkpoint inhibitor(s)” as used herein refers to molecules that completely or partially reduce, inhibit, interfere with or modulate the activity of one or more checkpoint proteins. Checkpoint proteins regulate T-cell activation or function. Checkpoint proteins include, but are not limited to CTLA-4 and its ligands CD80 and CD86; PD-1 and its ligands PDL1 and PDL2; LAGS, B7-H3, B7-114, TIM3, ICOS, and BTLA (Pardoll et al. Nature Reviews Cancer 12: 252-264 (2012)).

As used herein, the terms “individual”, “patient”, or “subject” can be an individual organism, a vertebrate, a mammal, or a human. In some embodiments, the individual, patient or subject is a human.

As used herein, the terms “linker,” “spacer” or “epitope flanking sequence” refer to any amino acid sequence comprising from a total of 1 to 200 amino acid residues; or about 1 to 10 amino acid residues, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times.

As used herein the term “neoepitope” or “neo antigen” is understood in the art to refer to an epitope that emerges or develops in a subject after exposure to or occurrence of a particular event (e.g., development or progression of a particular disease, disorder or condition, e.g., infection, cancer, stage of cancer, etc). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neoepitope is one that triggers an immune response that kills or otherwise destroys cells that express it (e.g., at a relevant level) In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope).

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The term “vaccine” as used herein is a preparation used to enhance protective immunity against cancer, or infectious agents such as viruses, fungi, bacteria and other pathogens. A vaccine may be useful as a prophylactic agent or a therapeutic agent. Vaccines contain cells or antigens which, when administered to the body, induce an immune response with the production of antibodies and immune lymphocytes (T-cells and B-cells).

### Vaccines of the Present Technology

In one aspect, the present disclosure provides a vaccine including a polypeptide comprising (i) an oligomer of at least one tumor-specific peptide epitope recognized by CD8 T cells and (ii) an oligomer of at least one tumor-specific peptide epitope recognized by CD4 T cells, wherein the oligomer of the at least one tumor-specific peptide epitope recognized by CD8 T cells is linked to the oligomer of the at least one tumor-specific peptide epitope recognized by CD4 T cells via a linker, and wherein the oligomer of the at least one tumor-specific peptide epitope recognized by CD4 T cells is at least a dimer. In another aspect, the present disclosure provides a vaccine including a nucleic acid molecule encoding a polypeptide comprising (i) an oligomer of at least one tumor-specific peptide epitope recognized by CD8 T cells and (ii) an oligomer of at least one tumor-specific peptide epitope recognized by CD4 T cells, wherein the oligomer of the at least one tumor-specific peptide epitope recognized by CD8 T cells is linked to the oligomer of the at least one tumor-specific peptide epitope recognized by CD4 T cells via a linker, and wherein the oligomer of the at least one tumor-specific peptide epitope recognized by CD4 T cells is at least a dimer. The oligomer of the at least one tumor-specific peptide epitope recognized by CD8 T cells and/or the oligomer of the at least one tumor-specific peptide epitope recognized by CD4 T cells may be a dimer, a trimer, a tetramer, a pentamer, a hexamer, a heptamer, an octamer, a nonamer, a decamer, an 11-mer, a 12-mer, a 13-mer, a 14-mer or a 15-mer. Additionally or alternatively, in some embodiments, the oligomer of the at least one tumor-specific peptide epitope recognized by CD8 T cells comprises a single tumor-specific peptide epitope. In other embodiments, the oligomer of the at least one tumor-specific peptide epitope recognized by CD8 T cells comprises at least 2, at least 3, at least 4, at least 5, at least 6, at least 7, at least 8, at least 9, at least 10, at least 11, at least 12, at least 13, at least 14 or at least 15 distinct tumor-specific peptide epitopes. Additionally or alternatively, in certain embodiments, the oligomer of the at least one tumor-specific peptide epitope recognized by CD4 T cells comprises a single tumor-specific peptide epitope. In other embodiments, the oligomer of the at least one tumor-specific peptide epitope recognized by CD4 T cells comprises at least 2, at least 3, at least 4, at least 5, at least 6, at least 7, at least 8, at least 9, at least 10, at least 11, at least 12, at least 13, at least 14 or at least 15 distinct tumor-specific peptide epitopes.

In one aspect, the present disclosure provides a vaccine comprising at least two tandem repeats of a polypeptide, wherein the polypeptide comprises (i) at least one tumor-specific peptide epitope recognized by CD8 T cells, and (ii) at least one tumor-specific peptide epitope recognized by CD4 T cells, wherein the at least one tumor-specific peptide epitope recognized by CD8 T cells is linked to the at least one tumor-specific peptide epitope recognized by CD4 T cells via a linker. Also disclosed herein is a vaccine including a nucleic acid molecule encoding at least two tandem repeats of a polypeptide, wherein the polypeptide comprises (i) at least one tumor-specific peptide epitope recognized by CD8 T cells, and (ii) at least one tumor-specific peptide epitope recognized by CD4 T cells, wherein the at least one tumor-specific peptide epitope recognized by CD8 T cells is linked to the at least one tumor-specific peptide epitope recognized by CD4 T cells via a linker. In some embodiments, the vaccine comprises at least 2, at least 3, at least 4, at least 5, at least 6, at least 7, at least 8, at least 9, at least 10, at least 11, at least 12, at least 13, at least 14, or at least 15 tandem repeats of the polypeptide. In some embodiments, the at least one tumor-specific peptide epitope recognized by CD4 T cells and/or the at least one tumor-specific peptide epitope recognized by CD8 T cells is a monomer or an oligomer. Additionally or alternatively, in certain embodiments of the vaccine, each tandem repeat of the polypeptide comprises the same tumor-specific peptide epitope recognized by CD8 T cells and/or the same tumor-specific peptide epitope recognized by CD4 T cells. In other embodiments, at least one of the at least two tandem repeats of the polypeptide comprise distinct tumor-specific peptide epitopes recognized by CD8 T cells and/or distinct tumor-specific peptide epitopes recognized by CD4 T cells.

In any of the preceding embodiments of the vaccine disclosed herein, the nucleic acid molecule comprises RNA or DNA. Additionally or alternatively, in some embodiments of the vaccines disclosed herein, the linker is a cleavable peptide linker or a rigid peptide linker. Examples of suitable linkers include, but are not limited to a GS4 linker (SEQ ID NO: 336), a GS3 linker (SEQ ID NO: 337), a P2A linker, a T2A linker, an E2A linker, a F2A linker, a BmCPV2A linker, an AAY linker, a GPGPG linker (SEQ ID NO: 338), an EAAAK linker (SEQ ID NO: 339), a HEYGAEALERAG linker (SEQ ID NO: 340), a KK linker, or an RVRR linker (SEQ ID NO: 341). In certain embodiments, the linker comprises an ubiquitination sequence. Other linkers, spacers and flanking sequences that are known in the art to enhance epitope presentation and immunogenicity may also be included in the vaccines of the present technology.

The nucleic acid cancer vaccines of the disclosure may encode one or more tumor-specific peptide epitopes (which are portions of personalized cancer antigens). Portions of personalized cancer antigens are segments of personalized cancer antigens that are less than the full-length personalized cancer antigen. In one embodiment, the nucleic acid cancer vaccine is composed of open reading frames that may contain any number of tumor-specific peptide epitopes. In some embodiments the nucleic acid cancer vaccine is composed of open reading frames encoding 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 45 or more, 50 or more, 55 or more, 60 or more, 65 or more, 70 or more, 75 or more, 80 or more, 85 or more, 90 or more, 95 or more, 100 or more, 105 or more, 110 or more, 115 or more, 120 or more, 125 or more, 130 or more, 135 or more, 140 or more, 145 or more, 150 or more, 155 or more, 160 or more, 165 or more, 170 or more, 175 or more, 180 or more, 185 or more, 190 or more, 195 or more, or 200 or more tumor-specific peptide epitopes. In other embodiments the nucleic acid cancer vaccine is composed of open reading frames encoding 200 or less, 195 or less, 190 or less, 185 or less, 180 or less, 175 or less, 170 or less, 165 or less, 160 or less, 155 or less, 150 or less, 145 or less, 140 or less, 135 or less, 130 or less, 125 or less, 120 or less, 115 or less, 110 or less, 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, or 10 or less, or 5 or less tumor-specific peptide epitopes. In other embodiments the nucleic acid cancer vaccine is composed of open reading frames encoding up to 200, up to 195, up to 190, up to 185, up to 180, up to 175, up to 170, up to 165, up to 160, up to 155, up to 150, up to 145, up to 140, up to 135, up to 130, up to 125, up to 120, up to 115, up to 110, up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, up to 10, up to 5, or up to 4 tumor-specific peptide epitopes. In certain embodiments, the nucleic acid cancer vaccines described herein include open reading frames that encode tumor-specific epitopes or antigens based on specific mutations (neoepitopes) and/or those expressed by cancer-germline genes (antigens common to tumors found in multiple patients).

Each tumor-specific peptide epitope of the vaccines of the present technology (e.g., tumor-specific peptide epitopes recognized by CD8 T cells or CD4 T cells) may be any length that is reasonable for an epitope. In some embodiments, the length of each tumor-specific peptide epitope (e.g., tumor-specific peptide epitopes recognized by CD8 T cells or CD4 T cells) is not necessarily equal. In some embodiments, each tumor-specific peptide epitope (e.g., tumor-specific peptide epitopes recognized by CD8 T cells or CD4 T cells) in a nucleic acid cancer vaccine is a different length. In certain embodiments, at least two (e.g., at least 3, at least 4, at least 5, at least 6, at least 7, at least 8, at least 9, at least 10, at least 11, at least 12, at least 13, at least 14, at least 15, and up to and including all) of the tumor-specific peptide epitopes (e.g., tumor-specific peptide epitopes recognized by CD8 T cells or CD4 T cells) in a nucleic acid cancer vaccine are different lengths. In some embodiments, the length of at least one of the tumor-specific peptide epitopes (e.g., tumor-specific peptide epitopes recognized by CD8 T cells or CD4 T cells) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 75, at least 80, at least 85, at least 90, at least 95, or at least 100 amino acids. In other embodiments, the length of at least one of the tumor-specific peptide epitopes (e.g., tumor-specific peptide epitopes recognized by CD8 T cells or CD4 T cells) is 100 or less, 95 or less, 90 or less, 85 or less, 80 or less, 75 or less, 70 or less, 65 or less, 60 or less, 55 or less, 50 or less, 45 or less, 40 or less, 35 or less, 30 or less, 25 or less, 20 or less, 15 or less, 14 or less, 13 or less, 12 or less, 11 or less, 10 or less, 9 or less, 8 or less, 7 or less, 6 or less, 5 or less, 4 or less, 3 or less, or 2 or less amino acids. In other embodiments, the length of at least one of the tumor-specific peptide epitopes (e.g., tumor-specific peptide epitopes recognized by CD8 T cells or CD4 T cells) is up to 100, up to 95, up to 90, up to 85, up to 80, up to 75, up to 70, up to 65, up to 60, up to 55, up to 50, up to 45, up to 40, up to 35, up to 30, up to 25, up to 20, up to 15, or up to 10 amino acids.

In some embodiments, each of the tumor-specific peptide epitopes (e.g., tumor-specific peptide epitopes recognized by CD8 T cells or CD4 T cells) encoded by the nucleic acid cancer vaccine may have a different length. In certain embodiments, at least one of the tumor-specific peptide epitopes (e.g., tumor-specific peptide epitopes recognized by CD8 T cells or CD4 T cells) has a different length than another tumor-specific peptide epitope encoded by the nucleic acid cancer vaccine. Each tumor-specific peptide epitope (e.g., tumor-specific peptide epitopes recognized by CD8 T cells or CD4 T cells) may be any length that is reasonable for an epitope.

In some embodiments, the percentages of lengths of the tumor-specific peptide epitopes (e.g., tumor-specific peptide epitopes recognized by CD8 T cells or CD4 T cells) encoded by the nucleic acid vaccines may be as follows: about 100%<15 amino acids, about 0%≥15 amino acids; about 95%<15 amino acids, about 5%≥15 amino acids; about 90%<15 amino acids, about 10%≥15 amino acids; about 85%<15 amino acids, about 15%≥15 amino acids; about 80%<15 amino acids, about 20%≥15 amino acids; about 75%<15 amino acids, about 25%≥15 amino acids; about 70%<15 amino acids, about 30%≥15 amino acids; about 65%<15 amino acids, about 35%≥15 amino acids; about 60%<15 amino acids, about 40%≥15 amino acids; about 55%<15 amino acids, about 45%≥15 amino acids; about 50%<15 amino acids, about 50%≥15 amino acids; about 45%<15 amino acids, about 55% 15 amino acids; about 40%<15 amino acids, about 60%≥15 amino acids; about 35%<15 amino acids, about 65%≥15 amino acids; about 30%<15 amino acids, about 70%≥15 amino acids; about 25%<15 amino acids, about 75%≥15 amino acids; about 20%<15 amino acids, about 80%≥15 amino acids; about 15%<15 amino acids, about 85% 15 amino acids; about 10%<15 amino acids, about 90%≥15 amino acids; about 5%<15 amino acids, about 95%≥15 amino acids; or about 0%<15 amino acids, about 100%≥15 amino acids.

In some embodiments, the lengths of the tumor-specific peptide epitopes (e.g., tumor-specific peptide epitopes recognized by CD8 T cells or CD4 T cells) may be categorized in one of the following groups (for a total of 100%): 8-12 amino acids, 13-17 amino acids, 18-21 amino acids, 22-26 amino acids, or 27-31 amino acids. About 0%, 5%, 10%, 15%, 20%, 25%, 30%, 35%, 40%, 4%≥5% 50%, 55%, 60%, 65%, 70%, 75%, 80%, 85%, 90%, 95% or 100% of the tumor-specific peptide epitopes (e.g., tumor-specific peptide epitopes recognized by CD8 T cells or CD4 T cells) encoded by the open reading frames of the nucleic acids may be 8-12 amino acids in length. About 0%, 5%, 10%, 15%, 20%, 25%, 30%, 35%, 40%, 45% 50%, 55%, 60%, 65%, 70%, 75%, 80%, 85%, 90%, 95%, or 100% of the tumor-specific peptide epitopes (e.g., tumor-specific peptide epitopes recognized by CD8 T cells or CD4 T cells) encoded by the open reading frames of the nucleic acids may be 13-17 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the tumor-specific peptide epitopes (e.g., tumor-specific peptide epitopes recognized by CD8 T cells or CD4 T cells) encoded by the open reading frames of the nucleic acids may be 18-21 amino acids in length. About 0%, 5%, 10%, 15%, 20%, 25%, 30%, 35%, 40%, 45%, 50%, 55%, 60%, 65%, 70%, 75%, 80%, 85%, 90%, 95% or 100% of the tumor-specific peptide epitopes (e.g., tumor-specific peptide epitopes recognized by CD8 T cells or CD4 T cells) encoded by the open reading frames of the nucleic acids may be 22-26 amino acids in length. About 0%, 5%, 10%, 15%, 20%, 25%, 30%, 35%, 40%, 45%, 50%, 55%, 60%, 65%, 70%, 75%, 80%, 85%, 90%, 95%, or 100% of the tumor-specific peptide epitopes (e.g., tumor-specific peptide epitopes recognized by CD8 T cells or CD4 T cells) encoded by the open reading frames of the nucleic acids may be 27-31 amino acids in length. Several non-limiting examples of the percentages of tumor-specific peptide epitope lengths encoded by the open reading frames of the nucleic acids follow.

In any of the preceding embodiments of the vaccine disclosed herein, the at least one tumor-specific peptide epitope recognized by CD8 T cells is selected from among: AEPINIQTW (SEQ ID NO: 7), FPSDSWCYF (SEQ ID NO: 8), SYLDSGIIF (SEQ ID NO: 9), ACDPHSGHFV (SEQ ID NO: 10), AVCPWTWLRG (SEQ ID NO: 11), ILDKVLVHL (SEQ ID NO: 12), TLDWLLQTPK (SEQ ID NO: 13), RPHVPESAF (SEQ ID NO: 14), KIFSEVTLK (SEQ ID NO: 15), SHETVIIEL (SEQ ID NO: 16), KILDAVVAQK (SEQ ID NO: 17), FLEGNEVGKTY (SEQ ID NO: 18), EEKLIVVLF (SEQ ID NO: 19), SELFRSGLDSY (SEQ ID NO: 20), FRSGLDSYV (SEQ ID NO: 21), EAFIQPITR (SEQ ID NO: 22), KINKNPKYK (SEQ ID NO: 23), ILDTAGREEY (SEQ ID NO: 24), KELEGILLL (SEQ ID NO: 25), ETVSEQSNV (SEQ ID NO: 26), QQITKTEV (SEQ ID NO: 27), FIASNGVKLV (SEQ ID NO: 28), FLDEFMEGV (SEQ ID NO: 29), SLFEGIDIYT (SEQ ID NO: 30), VESEDIAEL (SEQ ID NO: 31), RTKVVQTLW (SEQ ID NO: 32), IPIDGIFFT (SEQ ID NO: 33), KMIGNHLWV (SEQ ID NO: 34), AMFWSVPTV (SEQ ID NO: 35), CLNEYHLFL (SEQ ID NO: 36), KLMNIQQKL (SEQ ID NO: 37), QLSCISTYV (SEQ ID NO: 38), FLYNLLTRV (SEQ ID NO: 39), IILVAVPHV (SEQ ID NO: 40), HLYASLSRA (SEQ ID NO: 41), MLGEQLFPL (SEQ ID NO: 42), RLFPGLTIKI (SEQ ID NO: 43), TRSSGSHFVF (SEQ ID NO: 44), LRTKVYAEL (SEQ ID NO: 45), LLYQELLPL (SEQ ID NO: 46), IYKAPCENW (SEQ ID NO: 47), YYPPSQIAQL (SEQ ID NO: 48), LYNGMEHLI (SEQ ID NO: 49), HSVSSAFKK (SEQ ID NO: 50), YPPPPPALL (SEQ ID NO: 51), KVDPIGHVY (SEQ ID NO: 52), LMKVDPIGHVY (SEQ ID NO: 53), KVDPIGHVYF (SEQ ID NO: 54), FVVPYMIYLL (SEQ ID NO: 55), VSVQIISCQY (SEQ ID NO: 56), VQIISCQY (SEQ ID NO: 57), CVRVSGQGL (SEQ ID NO: 58), APARLERRHSA (SEQ ID NO: 59), AYHSIEWAI (SEQ ID NO: 60), YHSIEWAI (SEQ ID NO: 61), NAYHSIEWAI (SEQ ID NO: 62), KSQREFVRR (SEQ ID NO: 63), MRMNQGVCC (SEQ ID NO: 64), FLSDHLYLV (SEQ ID NO: 65), KPSDTPRPVM (SEQ ID NO: 66), HIAKSLFEV (SEQ ID NO: 67), AGQHIAKSLF (SEQ ID NO: 68), KPFCVLISL (SEQ ID NO: 69), RPHHDQRSL (SEQ ID NO: 70), SSYTGFANK (SEQ ID NO: 71), FLLDEAIGL (SEQ ID NO: 72), ALDPHSGHFV (SEQ ID NO: 73), HSCVMASLR (SEQ ID NO: 74), HDLGRLHSC (SEQ ID NO: 75), VSKILPSTW (SEQ ID NO: 76), GVADVLLYR (SEQ ID NO: 77), TESPFEQHI (SEQ ID NO: 78), GLEREGFTF (SEQ ID NO: 79), YTDFHCQYV (SEQ ID NO: 80), CILGKLFTK (SEQ ID NO: 81), GLFGDIYLA (SEQ ID NO: 82), SLADEAEVYL (SEQ ID NO: 83), KTLTSVFQK (SEQ ID NO: 84), ILNAMIAKIJ (SEQ ID NO: 85), FAFQEYDSF (SEQ ID NO: 86), LLDIVAPK (SEQ ID NO: 87), SPMIVGSPW (SEQ ID NO: 88), ASNASSAAK (SEQ ID NO: 89), CYMEAVAL (SEQ ID NO: 90), SIY, SVGDFSQEF (SEQ ID NO: 100), VSVGDFSQEF (SEQ ID NO: 101), KLKFVTLVF (SEQ ID NO: 102), VLAKKLKFV (SEQ ID NO: 103), FLFQDSKKI (SEQ ID NO: 104), NSKKKWFLF (SEQ ID NO: 105), VQKVASKIPF (SEQ ID NO: 106), ALFASRPRF (SEQ ID NO: 107), RFLEYLPLRF (SEQ ID NO: 108), TELERFLEY (SEQ ID NO: 109), LLHTELERF (SEQ ID NO: 110), LLHTELERFL (SEQ ID NO: 111), TLFHTFYEL (SEQ ID NO: 112), TLFHTFYELL (SEQ ID NO: 113), LFHTFYELLI (SEQ ID NO: 114), LFHTFYELL (SEQ ID NO: 115), TTLFHTFYEL (SEQ ID NO: 116), KFGDLTNNF (SEQ ID NO: 117), KLFESKAEL (SEQ ID NO: 118), KLFESKAELA (SEQ ID NO: 119), YNSFSSAPM (SEQ ID NO: 120), SFSSAPMPQI (SEQ ID NO: 121), GIPENSFNV (SEQ ID NO: 122), SVGDFSQEF (SEQ ID NO: 123), VSVGDFSQEF (SEQ ID NO: 124), TPAAPTAMA (SEQ ID NO: 125), FPGNQWNPV (SEQ ID NO: 126), LADFRLARLY (SEQ ID NO: 127), NHDETSFLL (SEQ ID NO: 128), TPAHPSQGA (SEQ ID NO: 129), TPAHPSQGAV (SEQ ID NO: 130), VTEKLQPTY (SEQ ID NO: 131), HPAPPAPPPA (SEQ ID NO: 132), VPKEHPAPPA (SEQ ID NO: 133), RPAARGSRV (SEQ ID NO: 134), FPKKIQMLA (SEQ ID NO: 135), FLDREQRESY (SEQ ID NO: 136), FPAAAFPTA (SEQ ID NO: 137), SPVTFPAAA (SEQ ID NO: 138), FPAAAFPTAS (SEQ ID NO: 139), ITDAHELGV (SEQ ID NO: 140), ITDAHELGVA (SEQ ID NO: 141), YTWPSGNIY (SEQ ID NO: 142), VLSSLVLVPL (SEQ ID NO: 143), NVLSSLVLV (SEQ ID NO: 144), SLPSNVLSSL (SEQ ID NO: 145), KIIAYQPYGK (SEQ ID NO: 146), RLMLRKVALK (SEQ ID NO: 147), QRLMLRKVAL (SEQ ID NO: 148), RLMLRKVAL (SEQ ID NO: 149), SLQRLMLRKV (SEQ ID NO: 150), ALQSQSISLV (SEQ ID NO: 151), ALQSQSISL (SEQ ID NO: 152), YLLFQNTDL (SEQ ID NO: 153), ALSPDGSIRK (SEQ ID NO: 154), FLLTDYALS (SEQ ID NO: 155), LLFAPEYGPK (SEQ ID NO: 156), WRNILLLSLH (SEQ ID NO: 157), SLHKGSLYPR (SEQ ID NO: 158), TLLSQVNKV (SEQ ID NO: 159), VRTLLSQVNK (SEQ ID NO: 160), RTAPRPGSQK (SEQ ID NO: 161), SLLRAAFFGK (SEQ ID NO: 162), LRAAFFGKCF (SEQ ID NO: 163), LRFNLIANQH (SEQ ID NO: 164), KLNFRLFVI (SEQ ID NO: 165), RKLNFRLFVI (SEQ ID NO: 166), KLNFRLFVIR (SEQ ID NO: 167), RIYTGEKPFK (SEQ ID NO: 168), FEAEFTQVA (SEQ ID NO: 169), ILMHGLVSL (SEQ ID NO: 170), RRNDDKSILM (SEQ ID NO: 171), SILMHGLVSL (SEQ ID NO: 172), RRWSALVIGL (SEQ ID NO: 173), KRRWSALVI (SEQ ID NO: 174), RRWSALVIG (SEQ ID NO: 175), STTKRRWSAL (SEQ ID NO: 176), YMASEVVEV (SEQ ID NO: 177), YMASEVVEVF (SEQ ID NO: 178), DEQIESMTY (SEQ ID NO: 179), KQAKVVNPPI (SEQ ID NO: 180), FMMPRIVDV (SEQ ID NO: 181), FMMPRIVDVT (SEQ ID NO: 182), MTFSSTKDYV (SEQ ID NO: 183), NEVSEVTVF (SEQ ID NO: 184), SQFARVPGYV (SEQ ID NO: 185), YVGSPLAAM (SEQ ID NO: 186), SQFARVPGY (SEQ ID NO: 187), WLVDLLPST (SEQ ID NO: 188), EEFWLVDLL (SEQ ID NO: 189), EEFWLVDLLP (SEQ ID NO: 190), HLYAYHEEL (SEQ ID NO: 191), EELSATVPS (SEQ ID NO: 192), EELSATVPSQ (SEQ ID NO: 193), FVADWAGTF (SEQ ID NO: 194), EESGGAVAFF (SEQ ID NO: 195), ESGGAVAFF (SEQ ID NO: 196), IPLSDNTIF (SEQ ID NO: 197), REFDKIELA (SEQ ID NO: 198), REFDKIELAY (SEQ ID NO: 199), SYQRYSHPLF (SEQ ID NO: 200), IYLHGSTDKL (SEQ ID NO: 201), YPVIFKSIM (SEQ ID NO: 202), KEYPVIFKSI (SEQ ID NO: 203), EYPVIFKSI (SEQ ID NO: 204), IFKSIMRQRL (SEQ ID NO: 205), YDYVSALHPV (SEQ ID NO: 206), HTHYDYVSAL (SEQ ID NO: 207), DYVSALHPV (SEQ ID NO: 208), FPQGLPNEY (SEQ ID NO: 209), LPNEYAFVTT (SEQ ID NO: 210), LPNEYAFVT (SEQ ID NO: 211), NEYAFVTTF (SEQ ID NO: 212), QGLPNEYAF (SEQ ID NO: 213), ALPQSILLF (SEQ ID NO: 214), TALPQSILLF (SEQ ID NO: 215), SPVLRSHSF (SEQ ID NO: 216), SYQLYTHPL (SEQ ID NO: 217), RFANPRDSF (SEQ ID NO: 218), TIIDNIKEM (SEQ ID NO: 219), LFSPGAANLF (SEQ ID NO: 220), FSPGAANLF (SEQ ID NO: 221), QPPALSPSY (SEQ ID NO: 222), APSPGQPPAL (SEQ ID NO: 223), FPSQRTSWEF (SEQ ID NO: 224), MPFTTVSELM (SEQ ID NO: 225), SELMKVSAM (SEQ ID NO: 226), HQFHVHPLL (SEQ ID NO: 227), MTITSRGTTV (SEQ ID NO: 228), RYGIRGFSTI (SEQ ID NO: 229), YGIRGFSTI (SEQ ID NO: 230), MAGPKGFQY (SEQ ID NO: 231), DMKARQKAL (SEQ ID NO: 232), DMKARQKALV (SEQ ID NO: 233), TRKNKKLAL (SEQ ID NO: 234), QTRKNKKLAL (SEQ ID NO: 235), LFRIKFKEPL (SEQ ID NO: 236), ISDRFIGIY (SEQ ID NO: 237), EISDRFIGIY (SEQ ID NO: 238), EISDRFIGI (SEQ ID NO: 239), QTSIQSPSLY (SEQ ID NO: 240), TSIQSPSLY (SEQ ID NO: 241), EMKRVFGFPV (SEQ ID NO: 242), VVDFKKNLEY (SEQ ID NO: 243), AAQARLQPV (SEQ ID NO: 244), HLARHRHLM (SEQ ID NO: 245), SPHLARHRHL (SEQ ID NO: 246), LLDKFVEWY (SEQ ID NO: 247), MPAWRTRGAI (SEQ ID NO: 248), MPAWRTRGA (SEQ ID NO: 249), LPVTRKNMPL (SEQ ID NO: 250), WTNCILHEY (SEQ ID NO: 251), HTLGAASSFM (SEQ ID NO: 252), HTLGAASSF (SEQ ID NO: 253), QLFARARPM (SEQ ID NO: 254), ISSQPQVPFY (SEQ ID NO: 255), SSQPQVPFY (SEQ ID NO: 256), NVELRRNVL (SEQ ID NO: 257), ESDLNSWPV (SEQ ID NO: 258), LPSFRPPTAL (SEQ ID NO: 259), ISIQRAQPL (SEQ ID NO: 260), ESIKEITNFK (SEQ ID NO: 261), SIKEITNFK (SEQ ID NO: 262), and ESIKEITNF (SEQ ID NO: 263).

In any of the foregoing embodiments of the vaccine disclosed herein, the at least one tumor-specific peptide epitope recognized by CD4 T cells is selected from among:

(SEQ ID NO: 91)
TLYQDDTLTLQAAGE,
(SEQ ID NO: 92)
YSVYFNLPADTIYTNH,
(SEQ ID NO: 93)
FSWAMDLDPKGAE
(SEQ ID NO: 94)
MIFEKHGFRRTTPP,
(SEQ ID NO: 95)
WRRAPAPGA,
(SEQ ID NO: 96)
PVTWRRAPA,
(SEQ ID NO: 97)
RVIKNSIRLTLE,
(SEQ ID NO: 98)
PYYFAAELPPRNLPEP,
(SEQ ID NO: 99)
GELIGILNAAKVPAD,
HEL,
(SEQ ID NO: 264)
DRSVLAKKLKFVTLVFRHGDRSPID,
(SEQ ID NO: 265)
NNSKKKWFLFQDSKKIQVEQPQ,
(SEQ ID NO: 266)
SPIKLVQKVASKIPFPDRITEESV,
(SEQ ID NO: 267)
TKRQVILLHTELERFLEYLPLRF,
(SEQ ID NO: 268)
SHTQTTLFHTFYELLIQKNKHK,
(SEQ ID NO: 269)
RLVLGKFGDLINNFSSPHAR,
(SEQ ID NO: 270)
LSPREEFLRLCKKIMMRSIQ,
(SEQ ID NO: 271)
PSTANYNSFSSAPMPQIPVASVTPT,
(SEQ ID NO: 272)
LCPREEFLRLCKKIMMRSIQ,
(SEQ ID NO: 273)
SHNELADSGIPENSFNVSSLVE,
(SEQ ID NO: 274)
SGSPPLRVSVGDFSQEFSPIQEAQQD,
(SEQ ID NO: 275)
RPAGRTQLLWTPAAPTAMAEVGPGHTP,
(SEQ ID NO: 276)
RGQIKLADFRLARLYSSEESR,
(SEQ ID NO: 277)
DEQGREAELARSGPSAAGPVRLKPGLVPGL,
(SEQ ID NO: 278)
AAVRPEQRPAARGSRV,
(SEQ ID NO: 279)
TFPKKIQMLARDFLDEY,
(SEQ ID NO: 280)
PETGEIQVKTFLDREQRESYELKV,
(SEQ ID NO: 281)
PGGDSGELITDAHELGVAHPPGY,
(SEQ ID NO: 282)
EVVGGYTWPSGNIYQGYWAQGKR,
(SEQ ID NO: 283)
TIKNSDKNVVLEHFG,
(SEQ ID NO: 284)
TRNSFALVPSLQRLMLRKVALKNVDSSPS,
(SEQ ID NO: 285)
SSHYKFSKPALQSQSISLVQQS,
(SEQ ID NO: 286)
TETVNHHYLLFQNTDLGSFHDLLR,
(SEQ ID NO: 287)
DRASFLLTDYALSPDGSIRKATG,
(SEQ ID NO: 288)
ERFWRNILLLSLHKGSLYPRIPGLGKE,
(SEQ ID NO: 289)
RGRLPAGAVRTLLSQVNKVWDQSS,
(SEQ ID NO: 290)
GHEHQPDMQKSLLRAAFFGKCFLDR,
(SEQ ID NO: 291)
ELQYRGRELRFNLIANQHLLAPGFVSETR,
(SEQ ID NO: 292)
EDLDANLRKLNFRLFVIRGQPAD,
(SEQ ID NO: 293)
GHQKLPGKIHLFEAEFTQVAKKEPDG,
(SEQ ID NO: 294)
TTPSGSAEYMASEVVEVFTDQAT,
(SEQ ID NO: 295)
SVLREDLGQLEYKYQYAYFRMGIKHPD,
(SEQ ID NO: 296)
PENDDLFMMPRIVDVTSLATEGG,
(SEQ ID NO: 297)
TLDDIKEWLEDEGQVLNIQMRRTLHK,
(SEQ ID NO: 298)
GRMSPSQFARVPGYVGSPLAAMNPK,
(SEQ ID NO: 299)
KAHVEGDGVVEEIIRYHPFLYDRET,
(SEQ ID NO: 300)
DGVSEEFWLVDLLPSTHYT,
(SEQ ID NO: 301)
DSYHLYAYHEELSATVPSQWKKIG,
(SEQ ID NO: 302)
GDQYKATDFVADWAGTFKMVFTPKDGSG,
(SEQ ID NO: 303)
EYWKVLDGELEVAPEYPQSTARDWL,
(SEQ ID NO: 304)
TTTSVKKEELVLSEEDFQGITPGAQ,
(SEQ ID NO: 305)
SLTEESGGAVAFFPGNLSTSSSA,
(SEQ ID NO: 306)
KLRTIPLSDNTIFRRICTIAKHLE,
(SEQ ID NO: 307)
SHHTHSYQRYSHPLFLPGHRLDPPI,
(SEQ ID NO: 308)
DVTGPHLYSIYLHGSTDKLPYVTMGS,
(SEQ ID NO: 309)
ARLQSKEYPVIFKSIMRQRLISPQL,
(SEQ ID NO: 310)
LHTHYDYVSALHPVSTPSKEYTSA,
(SEQ ID NO: 311)
SDAFSGLTALPQSILLFGP,
(SEQ ID NO: 312)
SHQIHSYQLYTHPLLHPWDHRD,
(SEQ ID NO: 313)
STQHADLTIIDNIKEMNFLRRYK,
(SEQ ID NO: 314)
ASATEPANDSLFSPGAANLFSTYLAR,
(SEQ ID NO: 315)
AASAAAFPSQRTSWEFLQSLVSIKQEK,
(SEQ ID NO: 316)
GSVLQFMPFTTVSELMKVSAMSSPKV,
(SEQ ID NO: 317)
DKGHQFHVHPLLHSGDDLDP,
(SEQ ID NO: 318)
NQVLASRYGIRGFSTIKIFQKGESPV,
(SEQ ID NO: 319)
MAGPKGFQYRALYPFRRER,
(SEQ ID NO: 320)
VTLNDMKARQKALVRERERQLA,
(SEQ ID NO: 321)
SRLQTRKNKKLALSSTPSNIAPSD,
(SEQ ID NO: 322)
LNTGLFRIKFKEPLENLI,
(SEQ ID NO: 323)
SLRNNMFEISDRFIGIYKTYNITK,
(SEQ ID NO: 324)
WCTEMKRVFGFPVHYTDVSNMS,
(SEQ ID NO: 325)
VKQLERGEASVVDFKKNLEYAAT,
(SEQ ID NO: 326)
STEVEPKESPHLARHRHLMKTLVKSLST,
(SEQ ID NO: 327)
LMSNLAFADFCMRMYL,
(SEQ ID NO: 328)
TKLKSKAPHWTNCILHEYKNLSTS,
(SEQ ID NO: 329)
PAAGDFIRFRFFQLLRLERFF,
(SEQ ID NO: 330)
YLSHTLGAASSFMRPTVPPPQF,
(SEQ ID NO: 331)
ALLQNVELRRNVLVSPTPLAN,
(SEQ ID NO: 332)
FAKGFRESDLNSWPVAPRPLLSV,
(SEQ ID NO: 333)
GLTRISIQRAQPLPPCLPSFRPPTALQGLS,
(SEQ ID NO: 334)
TGKPEMDFVRLAQLFARARPMGLF,
and
(SEQ ID NO: 335)
DGAWPVLLDKFVEWYKDKQMS.

In any and all embodiments of the vaccine disclosed herein, the at least one tumor-specific peptide epitope recognized by CD8 T cells and/or the at least one tumor-specific peptide epitope recognized by CD4 T cells is a tumor antigen. Examples of tumor-specific peptide epitopes that are derived from one or more tumor antigens and recognized by CD8 T cells include but are not limited to MAGE, BAGE, GAGE, NY-ESO-1, Tyrosinase, Melan-A, gp100, CEA, MART-1, HER2, WT1, MUC1, ppCT, Beta-catenin, CDK4, LPGAT1, CASP-8, CDKN2A, HLA-A11d, CLPP, GPNMB, RBAF600, SIRT2, SNRPD1, SNRP116, MART2, MUM-1f, MUM-2, MUM-3, Myosin class I, N-ras, OS-9, Elongation factor 2, NFYC, Alpha-actinin-4, Malic enzyme, HLA-A2, Hsp70-2, SETDB1, METTL17, ALDH1A1, CDKN2A, TKT, SEC24A, EXOC8, MRPS5, PABPC1, KIF2C, POLA2, CCT6A, TRRAP, DNMT1, PABPC3, MAGE-A10, FMN2, TMEM48, AKAP13, OR8B3, WASL, MAGEA6, PDS5A, MED13, FLNA, KIB1B, KFI1BP, NARFL, PPFIA4, CDC37L1, MLL3, FLNA, DOPEY2, TTBK2, KIF26B, SPOP, RETSAT, CLINT1, COX7A2, FAM3C, CSMD1, PPP1R3B, CDK12, CSNK1A1, GAS7, MATN, HAUS3, MTFR2, CHTF18, MYADM, HERC1 and HSDL1. Examples of tumor-specific peptide epitopes that are derived from one or more tumor antigens and recognized by CD4 T cells include but are not limited to COA-1, ARTC1, CDC27, FN1, LDLR-FUT fusion protein, neo-PAP, PTPRK and Triosephosphate isomerase.

Additionally or alternatively, in some embodiments, the at least one tumor-specific peptide epitope recognized by CD8 T cells and/or the at least one tumor-specific peptide epitope recognized by CD4 T cells is a personalized neoantigen (or neoepitope) specific for a cancer subject. A personalized neoantigen (or neoepitope) is present in a tumor of an individual and is not expressed or expressed at low levels in normal non-cancerous tissue of the individual. The personalized neoantigen may or may not be present in tumors of other individuals.

In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations.

In some embodiments the nucleic acid cancer vaccines described herein may include tumor-specific peptide epitopes or antigens based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

Epitopes can be identified using a free or commercial database (Lonza Epibase, antitope for example). Such tools are useful for predicting the most immunogenic epitopes within a target antigen protein. The selected peptides may then be synthesized and screened in human HLA panels, and the most immunogenic sequences are used to construct the nucleic acids encoding the peptide epitope(s). One strategy for mapping epitopes of Cytotoxic T-Cells based on generating equimolar mixtures of the four C-terminal peptides for each nominal 11-mer across a protein. This strategy would produce a library antigen containing all the possible active CTL epitopes.

The neoepitopes may be designed to optimally bind to MHC in order to promote a robust immune response. In some embodiments each tumor-specific peptide epitope comprises an antigenic region and a MHC stabilizing region. An MHC stabilizing region is a sequence which stabilizes the peptide in the MHC. All of the MHC stabilizing regions within the tumor-specific epitopes may be the same or they may be different. The MHC stabilizing regions may be at the N terminal portion of the peptide or the C terminal portion of the peptide. Alternatively the MHC stabilizing regions may be in the central region of the peptide.

The MHC stabilizing region may be 5-10, 5-15, 8-10, 1-5, 3-7, or 3-8 amino acids in length. In yet other embodiments the antigenic region is 5-100 amino acids in length. The peptides interact with the molecules of MHC class I by competitive affinity binding within the endoplasmic reticulum, before they are presented on the cell surface. The affinity of an individual peptide is directly linked to its amino acid sequence and the presence of specific binding motifs in defined positions within the amino acid sequence. The peptide being presented in the MHC is held by the floor of the peptide-binding groove, in the central region of the α1/α2 heterodimer (a molecule composed of two nonidentical subunits). The sequence of residues of the peptide-binding groove's floor determines which particular peptide residues it binds.

Optimal binding regions may be identified by a computer assisted comparison of the affinity of a binding site (MHC pocket) for a particular amino acid at each amino acid in the binding site for each of the target epitopes to identify an ideal binder for all of the examined antigens. The MHC stabilization regions of the epitopes may be identified using amino acid prediction matrices of data points for a binding site. An amino acid prediction matrix is a table having a first and a second axis defining data points. Prediction matrices can be generated as shown in Singh, H. and Raghava, G. P. S. (2001), “ProPred: prediction of HLA-DR binding sites.” Bioinformatics, 17(12), 1236-37). In some embodiments, the prediction matrix is based on evolutionary conservation, in another embodiment, the prediction matrix uses physiochemical similarity to examine how similar a somatic amino acid is to the germline amino acid (e.g., Kim et al., J Immunol. 2017: 3360-3368). The similarity of the somatic amino acid to the germline amino acid approximates how a mutation affects binding (e.g., T cell receptor recognition). In some embodiments, less similarity is indicative of improved binding (e.g., T cell receptor recognition).

In some embodiments the MHC stabilizing region is designed based on the subject's particular MHC. In that way the MHC stabilizing region can be optimized for each patient.

In some embodiments, the neoepitopes are 13 residues or less in length and may consist of between about 8 and about 11 residues, particularly 9 or 10 residues. In other embodiments the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

Neoepitopes having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Be, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the alpha-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

Immunogenicity may be considered in the selection of optimal neoepitopes for inclusion in a vaccine. As a set of non-limiting examples, immunogenicity may be assessed by analyzing the MHC binding capacity of a neoepitope, HLA promiscuity, mutation position, predicted T cell reactivity, actual T cell reactivity, structure leading to particular conformations and resultant solvent exposure, and representation of specific amino acids.

One important aspect of a neoepitope included in a vaccine is a lack of self-reactivity. The putative neoepitopes may be screened to confirm that the epitope is restricted to tumor tissue, for instance, arising as a result of genetic change within malignant cells. Ideally, the tumor-specific epitope should not be present in normal tissue of the patient and thus, self-similar epitopes are filtered out of the dataset. A personalized coding genome may be used as a reference for comparison of neoantigen candidates to determine lack of self-reactivity. In some embodiments, a personalized coding genome is generated from an individualized transcriptome and/or exome.

### Adoptive Cell Therapy

The presently disclosed subject matter provides methods of using adoptive cell therapeutic compositions for the treatment of a tumor. The adoptive cell therapeutic compositions of the presently disclosed subject matter can be cells of the lymphoid lineage or myeloid lineage. Examples of myeloid cells include but are not limited to, mast cells, monocytes, macrophages, dendritic cells, eosinophils, neutrophils, basophils. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells can be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells.

Natural killer (NK) cells can be lymphocytes that are part of cell-mediated immunity and act during the innate immune response. NK cells do not require prior activation in order to perform their cytotoxic effect on target cells.

The adoptive cell therapeutic compositions of the presently disclosed subject matter can be administered to a subject (e.g., a human subject) in need thereof for the treatment of cancer. In some embodiments, the immune cell is a lymphocyte, such as a T cell, a B cell or a natural killer (NK) cell. In certain embodiments, the adoptive cell therapeutic composition comprises T cells. The T cell can be a CD4⁺ T cell or a CD8⁺ T cell. In certain embodiments, the T cell is a CD4⁺ T cell. In certain embodiments, the T cell is a CD8⁺ T cell.

The presently disclosed adoptive cell therapeutic compositions of the present technology may further include at least one recombinant or exogenous co-stimulatory ligand. For example, the presently disclosed adoptive cell therapeutic compositions can be further transduced with at least one co-stimulatory ligand, such that the immune cells co-express or are induced to co-express the at least one co-stimulatory ligand. Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-a, CD134L/OX40L/CD252, CD27L/CD70, Fas ligand (FasL), CD30L/CD153, tumor necrosis factor beta (TNFP)/lymphotoxin-alpha (LTa), lymphotoxin-beta O-TO), CD257/B cell-activating factor (B AFF)/Bly s/THANK/Tall-1, glucocorticoid-induced TNF Receptor ligand (GITRL), and T F-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof.

Furthermore, the presently disclosed adoptive cell therapeutic compositions can further comprise at least one exogenous cytokine. For example, a presently disclosed adoptive cell therapeutic composition can be further transduced with at least one cytokine, such that the adoptive cell therapeutic compositions secrete the at least one cytokine. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The adoptive cell therapeutic compositions can be generated from peripheral donor lymphocytes, e.g., those disclosed in Sadelain, M., et al., Nat Rev Cancer 3:35-45 (2003), in Morgan, R. A. et al. (2006) Science 314: 126-129, in Panelli et al. (2000) J Immunol 164:495-504; Panelli et al. (2000) J Immunol 164:4382-4392 (2000), and in Dupont et al. (2005) Cancer Res 65:5417-5427; Papanicolaou et al. (2003) Blood 102:2498-2505. The adoptive cell therapeutic compositions (e.g., T cells) can be autologous, non-autologous (e.g., allogeneic), or derived in vitro from engineered progenitor or stem cells.

The unpurified source of immune cells can be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In some embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In some embodiments, the cells are collected in a medium comprising 2% fetal calf serum (FCS) or 0.2% bovine serum albumin (BSA) or any other suitable, preferably sterile, isotonic medium.

In some embodiments, the adoptive cell therapeutic compositions comprise one or more additional modifications. For example, in some embodiments, the adoptive cell therapeutic compositions comprise and express (is transduced to express) a chimeric co-stimulatory receptor (CCR). CCR is described in Krause et al. (1998) J. Exp. Med. 188(4):619-626, and US20020018783, the contents of which are incorporated by reference in their entireties. CCRs mimic co-stimulatory signals, but unlike, engineered receptors, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with an engineered receptor, can augment T-cell reactivity against the dual-antigen expressing T cells, thereby improving selective tumor targeting.

In some embodiments, the adoptive cell therapeutic compositions are further modified to suppress expression of one or more genes. In some embodiments, the adoptive cell therapeutic compositions are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the adoptive cell therapeutic compositions are modified to disrupt or reduce expression of an endogenous T-cell receptor gene (see, e.g. WO 2014153470, which is incorporated by reference in its entirety). In some embodiments, the adoptive cell therapeutic compositions are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

In some embodiments, the adoptive cell therapeutic compositions provided herein express a T-cell receptor (TCR) or other cell-surface ligand that binds to a target antigen, such as a tumor antigen. In some embodiments, the T cell receptor is a wild-type or native T-cell receptor. In some embodiments, the TCR is an engineered receptor or a non-native receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR).

In exemplary embodiments, the adoptive cell therapeutic compositions provided herein express a native receptor, a non-native receptor, or an engineered receptor (e.g., a CAR, caTCR, or eTCR) or other cell-surface ligand that binds to a tumor antigen. In some embodiments, the adoptive cell therapeutic compositions provided herein express a native receptor, a non-native receptor, or an engineered receptor (e.g., a CAR, caTCR, or eTCR) or other cell-surface ligand that binds to a tumor antigen presented in the context of an MIC molecule. In some embodiments, the adoptive cell therapeutic compositions provided herein express a native receptor, a non-native receptor or an engineered receptor (e.g., a CAR, caTCR, or eTCR) or other cell-surface ligand that binds to a tumor antigen presented in the context of an HLA-A2 molecule. In exemplary embodiments, the adoptive cell therapeutic compositions provided herein express a native receptor, a non-native receptor or an engineered receptor (e.g., a CAR, caTCR, or eTCR) or other cell-surface ligand that binds to a tumor antigen. Examples of tumor antigens bound by the native receptor, non-native receptor or engineered receptor (e.g., a CAR, caTCR, or eTCR) or other cell-surface ligand include, but is not limited to, carbonic anhydrase 9 (CAIX), CD19, prominin-1 (CD133), CD38 antigen (CD38), CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, and peptide MHC complexes (with peptides derived from TP53, KRAS, MYC, EBNA1-6, PRAME, MART, tyronsinase, MAGEA1-A6, pmel17, LMP2, or WT1). Exemplary engineered receptors that bind to CD19 are described in International Publication No. WO2017070608, which is incorporated by reference in its entirety.

### Vectors

Many expression vectors are available and known to those of skill in the art and can be used for expression of polypeptides provided herein, for example, for expression of a polypeptide comprising (i) an oligomer of at least one tumor-specific peptide epitope recognized by CD8 T cells and (ii) at least one tumor-specific peptide epitope recognized by CD4 T cells, wherein the oligomer is operably linked to the at least one tumor-specific peptide epitope recognized by CD4 T cells. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g. a hexa-his tag (SEQ ID NO: 342) or a myc tag, hemagglutinin tag or a tag for purification, for example, a GST fusion, and a sequence for directing protein secretion and/or membrane association.

Expression of the vaccine polypeptides disclosed herein can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the β-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of the vaccine polypeptides disclosed herein in host cells. A typical expression cassette contains a promoter operably linked to the nucleic acid sequence encoding the vaccine polypeptide of the present technology and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a vaccine polypeptide disclosed herein under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of mammalian cells can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the vaccine polypeptide can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide vaccine polypeptide expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. Mol. Cell. Biol. 5:431-437(1985)); PA317 (Miller, et al. Mol. Cell. Biol. 6:2895-2902(1986)); and CRIP (Danos, et al. Proc. Natd. Acad. Sci. USA 85:6460-6464(1988)). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al. Blood 80: 1418-1422(1992), or culturing with viral supernatant alone or concentrated vector stocks with or without appropriate growth factors and polycations, e.g., by the method of Xu, et al. Exp. Hemat. 22:223-230(1994); and Hughes, et al. J. Clin. Invest. 89: 1817(1992).

Transducing viral vectors can be used to express and/or secrete a cytokine (e.g., 4-1BBL and/or IL-12) in a vaccine polypeptide expressing cell. Preferably, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61 (1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed vaccine polypeptide is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J. Med. Sci. 298:278, (1989); Staubinger et al., Methods in Enzymology 101:512 (1983)), asialoorosomucoid-polylysine conjugation (Wu et al., Journal of Biological Chemistry 263: 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Polypeptides and Analogs and Polynucleotides

Also included in the presently disclosed subject matter are vaccine polypeptides, and polynucleotides encoding thereof that are modified in ways that enhance their anti-tumor activity when presented by an APC (e.g., macrophage, dendritic cell, B cell). The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (p) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the antineoplastic activity of the original vaccine polypeptide when presented by an APC (e.g., macrophage, dendritic cell, B cell). These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding the vaccine polypeptides disclosed herein can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Modes of Administration

Any method known to those in the art for contacting a cell, organ or tissue with one or more vaccine polypeptides, immune checkpoint inhibitors, T-cell-engaging multi-specific antibodies and/or adoptive cell therapeutic compositions disclosed herein may be employed. Suitable methods include in vitro, ex vivo, or in vivo methods. In vivo methods typically include the administration of one or more vaccine polypeptides, immune checkpoint inhibitors, T-cell-engaging multi-specific antibodies and/or adoptive cell therapeutic compositions to a mammal, suitably a human. When used in vivo, the one or more vaccine polypeptides, immune checkpoint inhibitors, T-cell-engaging multi-specific antibodies and/or adoptive cell therapeutic compositions described herein are administered to the subject in effective amounts (i.e., amounts that have desired therapeutic effect). The dose and dosage regimen will depend upon the degree of the disease state of the subject, the characteristics of the particular vaccine polypeptide, immune checkpoint inhibitor, T-cell-engaging multi-specific antibody and/or adoptive cell therapeutic composition used, e.g., its therapeutic index, and the subject's history.

The effective amount may be determined during pre-clinical trials and clinical trials by methods familiar to physicians and clinicians. An effective amount of one or more vaccine polypeptides, immune checkpoint inhibitors, T-cell-engaging multi-specific antibodies and/or adoptive cell therapeutic compositions useful in the methods may be administered to a mammal in need thereof by any of a number of well-known methods for administering pharmaceutical compounds. The vaccine polypeptides, immune checkpoint inhibitors, T-cell-engaging multi-specific antibodies and/or adoptive cell therapeutic compositions may be administered systemically or locally.

The one or more vaccine polypeptides, immune checkpoint inhibitors, T-cell-engaging multi-specific antibodies and/or adoptive cell therapeutic compositions described herein can be incorporated into pharmaceutical compositions for administration, singly or in combination, to a subject. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more vaccine polypeptides, immune checkpoint inhibitors, T-cell-engaging multi-specific antibodies and/or adoptive cell therapeutic compositions disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more vaccine polypeptides, immune checkpoint inhibitors, T-cell-engaging multi-specific antibodies and/or adoptive cell therapeutic compositions disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, vaccine polypeptide, immune checkpoint inhibitor, T-cell-engaging multi-specific antibody and/or adoptive cell therapeutic composition concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more vaccine polypeptides, immune checkpoint inhibitors, T-cell-engaging multi-specific antibodies and/or adoptive cell therapeutic compositions may be defined as a concentration of the agent at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

For therapeutic applications, a composition comprising a vaccine polypeptide, immune checkpoint inhibitor, T-cell-engaging multi-specific antibody and/or adoptive cell therapeutic composition disclosed herein, is administered to the subject.

In some embodiments, the vaccine polypeptide, immune checkpoint inhibitor, T-cell-engaging multi-specific antibody and/or adoptive cell therapeutic composition is administered one, two, three, four, or five times per day. In some embodiments, the vaccine polypeptide, immune checkpoint inhibitor, T-cell-engaging multi-specific antibody and/or adoptive cell therapeutic composition is administered more than five times per day. Additionally or alternatively, in some embodiments, the vaccine polypeptide, immune checkpoint inhibitor, T-cell-engaging multi-specific antibody and/or adoptive cell therapeutic composition is administered every day, every other day, every third day, every fourth day, every fifth day, or every sixth day. In some embodiments, the vaccine polypeptide, immune checkpoint inhibitor, T-cell-engaging multi-specific antibody and/or adoptive cell therapeutic composition is administered weekly, bi-weekly, tri-weekly, or monthly. In some embodiments, the vaccine polypeptide, immune checkpoint inhibitor, T-cell-engaging multi-specific antibody and/or adoptive cell therapeutic composition is administered for a period of one, two, three, four, or five weeks. In some embodiments, the vaccine polypeptide, immune checkpoint inhibitor, T-cell-engaging multi-specific antibody and/or adoptive cell therapeutic composition is administered for six weeks or more. In some embodiments, the vaccine polypeptide, immune checkpoint inhibitor, T-cell-engaging multi-specific antibody and/or adoptive cell therapeutic composition is administered for twelve weeks or more. In some embodiments, the vaccine polypeptide, immune checkpoint inhibitor, T-cell-engaging multi-specific antibody and/or adoptive cell therapeutic composition is administered for a period of less than one year. In some embodiments, the vaccine polypeptide, immune checkpoint inhibitor, T-cell-engaging multi-specific antibody and/or adoptive cell therapeutic composition is administered for a period of more than one year. In some embodiments, the vaccine polypeptide, immune checkpoint inhibitor, T-cell-engaging multi-specific antibody and/or adoptive cell therapeutic composition is administered throughout the subject's life.

In some embodiments of the methods of the present technology, the vaccine polypeptide, immune checkpoint inhibitor, T-cell-engaging multi-specific antibody and/or adoptive cell therapeutic composition is administered daily for 1 week or more. In some embodiments of the methods of the present technology, the vaccine polypeptide, immune checkpoint inhibitor, T-cell-engaging multi-specific antibody and/or adoptive cell therapeutic composition is administered daily for 2 weeks or more. In some embodiments of the methods of the present technology, the vaccine polypeptide, immune checkpoint inhibitor, T-cell-engaging multi-specific antibody and/or adoptive cell therapeutic composition is administered daily for 3 weeks or more. In some embodiments of the methods of the present technology, the vaccine polypeptide, immune checkpoint inhibitor, T-cell-engaging multi-specific antibody and/or adoptive cell therapeutic composition is administered daily for 4 weeks or more. In some embodiments of the methods of the present technology, the vaccine polypeptide, immune checkpoint inhibitor, T-cell-engaging multi-specific antibody and/or adoptive cell therapeutic composition is administered daily for 6 weeks or more. In some embodiments of the methods of the present technology, the vaccine polypeptide, immune checkpoint inhibitor, T-cell-engaging multi-specific antibody and/or adoptive cell therapeutic composition is administered daily for 12 weeks or more. In some embodiments, the vaccine polypeptide, immune checkpoint inhibitor, T-cell-engaging multi-specific antibody and/or adoptive cell therapeutic composition is administered daily throughout the subject's life.

### Formulations

Vaccine polypeptides, immune checkpoint inhibitors, T-cell-engaging multi-specific antibodies and/or adoptive cell therapeutic compositions of the presently disclosed subject matter can be provided systemically or directly to a subject for treating cancer. In certain embodiments, vaccine polypeptides, immune checkpoint inhibitors, T-cell-engaging multi-specific antibodies and/or adoptive cell therapeutic compositions are directly injected into an organ of interest. Additionally or alternatively, the vaccine polypeptides, immune checkpoint inhibitors, T-cell-engaging multi-specific antibodies and/or adoptive cell therapeutic compositions are provided indirectly to the organ of interest, for example, by administration into the circulatory system (e.g., the tumor vasculature) or into the tissue of interest (e.g., solid tumor). In certain embodiments, expansion and differentiation agents can be provided prior to, during or after administration of cells and compositions to increase production of the adoptive cell therapeutic compositions either in vitro or in vivo.

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

Adoptive cell therapeutic compositions of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising adoptive cell therapeutic compositions can comprise a purified population of cells. Those skilled in the art can readily determine the percentage of adoptive cell therapeutic compositions in a cell population using various well-known methods, such as fluorescence activated cell sorting (FACS). The ranges of purity in cell populations comprising adoptive cell therapeutic compositions can be from about 50% to about 55%, from about 55% to about 60%, about 60% to about 65%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The adoptive cell therapeutic compositions can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, pharmaceutical compositions comprise adoptive cell therapeutic compositions with a pharmaceutically acceptable carrier. Administration can be autologous or non-autologous. For example, adoptive cell therapeutic compositions can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering an adoptive cell therapeutic composition, it can be formulated in a unit dosage injectable form (solution, suspension, emulsion).

### Therapeutic Methods of the Present Technology

In one aspect, the present disclosure provides a method for treating cancer in a subject in need thereof comprising administering to the subject an effective amount of any and all embodiments of the vaccine described herein.

In another aspect, the present disclosure provides a method for treating cancer in a subject in need thereof comprising administering to the subject an effective amount of any and all embodiments of the vaccine described herein and sequentially, simultaneously or separately administering to the subject an effective amount of an immune checkpoint inhibitor. In some embodiments, the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Examples of immune checkpoint inhibitors include, but are not limited to, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

In one aspect, the present disclosure provides a method for treating cancer in a subject in need thereof comprising administering to the subject an effective amount of any and all embodiments of the vaccine described herein and sequentially, simultaneously or separately administering to the subject an effective amount of a T-cell-engaging multi-specific antibody. The T-cell-engaging multi-specific antibody may be a bispecific T cell engager (BiTE), a dual-affinity retargeting antibody (DART), a TandAb, a XmAb, a BITE-Fc, a 2:1 Crossmab, a duobody, a knobs-into-holes (KiH) antibody, or an IgG-scFv bispecific antibody. Additionally or alternatively, in some embodiments of the methods disclosed herein, the T-cell-engaging multi-specific antibody specifically targets one or more target antigens selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

In yet another aspect, the present disclosure provides a method for treating cancer in a subject in need thereof comprising administering to the subject an effective amount of any and all embodiments of the vaccine described herein and sequentially, simultaneously or separately administering to the subject an effective amount of an adoptive cell therapeutic composition (e.g., T cells), optionally wherein the adoptive cell therapeutic composition is obtained from a donor. The adoptive cell therapeutic compositions of the present technology comprise one or more of tumor infiltrating T cells, CD8+ T cells, CD4+ T cells, delta-gamma T-cells, and alpha-beta T-cells. In some embodiments, the adoptive cell therapeutic compositions provided herein express a T-cell receptor (TCR) or other cell-surface ligand that binds to a target antigen, such as a tumor antigen. In some embodiments, the T cell receptor is a wild-type or native T-cell receptor. In some embodiments, the TCR is an engineered receptor or a non-native receptor. In some embodiments, the engineered receptor is an engineered TCR (eTCR). In some embodiments, the engineered receptor is a chimeric antibody TCR (caTCR). In some embodiments, the engineered receptor is a chimeric antigen receptor (CAR). Additionally or alternatively, in some embodiments of the methods disclosed herein, the donor and the subject are the same or different.

In any of the preceding embodiments, the methods of the present technology further comprise administering a cytokine to the subject. The cytokine may be administered prior to, during, or subsequent to administration of the adoptive cell therapeutic composition. Examples of cytokines include, but are not limited to, interferon a, interferon β, interferon γ, complement C5a, IL-2, TNF alpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCRIO, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In any and all embodiments of the methods disclosed herein, the vaccine is administered pleurally, topically, parenterally, intravenously, subcutaneously, intranodally, intratumorally, intrathecally, intrapleurally or intraperitoneally. In any of the foregoing embodiments of the methods disclosed herein, the immune checkpoint inhibitor, the T-cell-engaging multi-specific antibody or the adoptive cell therapeutic composition is administered pleurally, topically, parenterally, intravenously, subcutaneously, intranodally, intratumorally, intrathecally, intrapleurally or intraperitoneally. In some embodiments, intratumoral administration comprises direct administration to a tumor or administration in close proximity to a tumor. In other embodiments, intratumoral administration comprises delivery via an oncolytic virus or an APC vaccine.

In any and all embodiments of the methods disclosed herein, the cancer is a carcinoma, sarcoma, a solid non-hematopoietic cancer, or a hematopoietic cancer. In certain embodiments of the methods disclosed herein, the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, glioblastomas, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

### Articles of Manufacture and Kits

The presently disclosed subject matter provides kits for the treatment or prevention of a neoplasia (e.g., solid tumor). In certain embodiments, the kit comprises a therapeutic or prophylactic composition containing an effective amount of any and all embodiments of the vaccine disclosed herein. In some embodiments, the kits further comprise at least one of an immune checkpoint inhibitor, a T-cell-engaging multi-specific antibody, and/or reagents for an adoptive cell therapeutic composition. Examples of reagents for an adoptive cell therapeutic composition include polynucleotides and vectors comprising TCR (i.e. heterologous T-cell receptor) constructs or CAR (i.e. chimeric antigen receptor) constructs. In some embodiments, the kit comprises a sterile container which contains the therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

Examples of immune checkpoint inhibitor may include an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. In some embodiments, the immune checkpoint inhibitor comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the T-cell-engaging multi-specific antibodies specifically target one or more target antigens selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1).

Additionally or alternatively, in some embodiments of the kits, the vaccine, the immune checkpoint inhibitor, the T-cell-engaging multi-specific antibody, and/or the adoptive cell therapeutic composition is formulated for intravenous, intramuscular, intraarterial, intrathecal, intracapsular, intraorbital, intradermal, intraperitoneal, transtracheal, subcutaneous, intracerebroventricular, oral or intranasal administration.

Optionally, the above described components of the kits of the present technology are packed in suitable containers and labeled for treatment or prevention of a neoplasia (e.g., solid tumor) in a subject. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, etc.

If desired, the kits can be provided together with instructions for administering the vaccine of the present technology to a subject having or at risk of developing a neoplasia (e.g., solid tumor). The instructions will generally include information about the use of the composition for the treatment or prevention of a neoplasia (e.g., solid tumor). In other embodiments, the instructions include at least one of the following: description of the therapeutic agent; dosage schedule and administration for treatment or prevention of a neoplasia (e.g., solid tumor) or symptoms thereof, precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The present technology is further illustrated by the following Examples, which should not be construed as limiting in any way.

### Example 1: Materials and Methods

### Mice

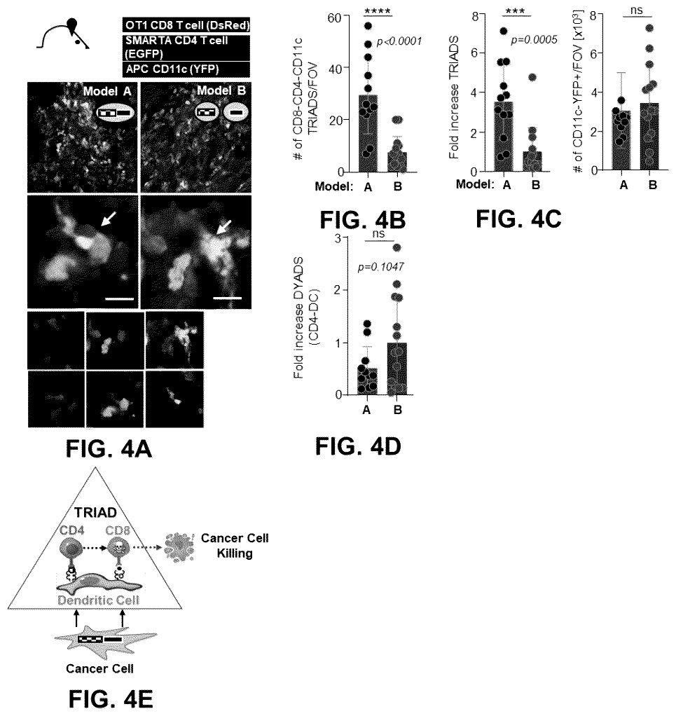

B6 mice (C57BL/6J), TCR_{OTI} r (C57BL/6-Tg(TcraTcrb)1100Mjb/J), TCR_{SMARTA} (B6.Cg-Ptprca Pepcb Tg(TcrLCMV)1Aox/PpmJ), CD11c-YFP (B6.Cg-Tg(Itgax-Venus)1Mnz/J), CD1 μl c-DTR-GFP (B6.FVB-1700016L21RikTg(Itgax-DTR/EGFP)57Lan/J), GFP transgenic (C57BL/6-Tg(CAG-EGFP)1Osb/J), B6 Thy1.1 (B6 PL-Thy1a/CyJ), and B6 CD45.1 (B6. SJL-Ptprca Pepcb/BoyJ) mice were purchased from the Jackson Laboratory. TCR_{SMARTA} mice were crossed to Thy. 1.1 mice to generate TCR_{SMARTA} Thy1.1 mice; for FIG. 4A imaging studies, TCR_{SMARTA} Thy1.1 mice were crossed to (FP-transgenic mice to generate TCR_{SMARTA} Thy1.1 GFP mice. TCR_{OTI} (Thy1.2) mice were crossed to CD45.1 mice to generate TCR_{OTI} CD45.1 mice. Both female and male mice were used for experimental studies. Donor and host mice were age- and sex-matched; mice were 7-12 weeks old. All mice were bred and maintained in the animal facility at Memorial Sloan Kettering Cancer Center (MSKCC). Experiments were performed in compliance with the MSKCC Institutional Animal Care and Use Committee (IACUC) regulations.

### Antibodies and Reagents

Fluorochrome-conjugated antibodies were purchased from BD Biosciences, eBioscience, and Biolegend. The OVA₂₅₇₋₂₆₄ and GP₆₁₋₈₀ peptides were purchased from GenScript.

### Intracellular Cytokine Staining

Intracellular cytokine staining was performed using the Foxp3 staining kit (BD Biosciences) following the manufacturer's protocol. Briefly, T cells isolated from lymph nodes or tumors were mixed with 3×10⁶ congenically marked B6 splenocytes and incubated with 1 μg/mL of OVA peptide and/or 2 ag/mL of GP peptide for 4-5 h at 37° C. in the presence of GolgiPlug (BD Biosciences). After staining for cell surface molecules, cells were fixed, permeabilized and stained with antibodies against IFNγ (XMG1.2) and TNFα (MP6-XT22).

### Flow Cytometric Analysis

Flow cytometric analysis was performed using Fortessa X20. Cells were sorted using BD FACS Aria (BD Biosciences) at the MSKCC Flow Core Facility. Flow data were analyzed with FlowJo v.10 software (Tree Star Inc.).

### Generation of Plasmids and Tumor Cell Lines

Tumor antigen-encoding pMFG-Cerulean vectors. pMFG-OVA₂₅₇₋₂₆₄-Cerulean, pMIFG-GP₆₁₋₈₀-Cerulean, and pMFG-OVA₂₅₇₋₂₆₄-GP₆₁₋₈₀-Ceruelan plasmids were constructed by inserting annealed oligonucleotides encoding triple SIINFEKL antigen (SEQ ID NO: 2)-AAY(linker) repeats (SIINFEKLAAY; SEQ ID NO: 3), GLKGPDIYKGVYQFKSVEFD (SEQ ID NO: 4), or (SIINFEKLAAY)₃-P2A-GLKGPDIYKGVYQFKSVEF) (SEQ ID NO: 5), respectively, into the NcoI-linearized pMFG-Cerulean vector, as previously described [45]. Restriction enzymes were purchased from New England Biolabs. All constructs were verified by sequence analysis. Phoenix packaging cells (ATCC) were transfected with pMFG constructs; supernatants were used to transduce B16-F10 mouse melanoma tumor cell line to generate B16-F10 OVA₂₅₇₋₂₆₄-Cerulean, B16-F10-GPF₆₁₋₈₀-Cerulean and B16-F10 OVA₂₅₇₋₂₆₄-GP₆₁₋₈₀-Cerulean, respectively [45]. Transduced bulk cell lines were sorted for similar Cerulean expression levels.

### In Vitro T Cell Activation

For the generation of effector TCR_{OT1} CD8 T cells and TCR_{SMARTA} CD14 T cells, single-cell suspensions were prepared from spleens of TCR_{OT1} and TCR_{SMARTA} transgenic mice and cultured in vitro in RPMI 1640 medium supplemented with 10% FBS, 100 IU/ml penicillin, 100 mg/ml streptomycin, nonessential amino acids, 1 mM sodium pyruvate, and 20 mM HEPES, together with 1 μg/mL of OVA₂₅₇₋₂₆₄ peptide or 2 μg/mL of GP₆₁₋₈₀ peptide, respectively, at a concentration of 4-5×10⁶ splenocytes/ml in the presence of 50 U/mL IL-2 for 4 days.

### Adoptive T Cell Transfer

For adoptive transfer studies, 2.5×10⁵ in vitro activated TCR_{OT1} (CD45.1) and/or 5×10⁵ in vitro activated TCR_{SMARTA} (Thy1.1) were transferred (i.v.) into tumor-bearing WT B6 mice at indicated time points post tumor transplantation (approximately 2-3 weeks post tumor implantation). Tumor-bearing mice were treated with cyclophosphamide (180 mg/kg), and 24 h later in vitro activated TCR_{OT1} CD8 T cells and/or TCR_{SMARTA} CD4 T cells were adoptively transferred. At indicated time points, adoptively transferred T cells were isolated from tumor-draining lymph nodes and tumors and prepared for downstream analyses.

### B16 and MICA 205 Transplantation Tumor Models

2.5×10⁶ B16 OVA₂₅₇₋₂₆₄-GP₆₁₋₈₀ (B16 OG) tumor cells, or a mixture of 1.25×10⁶ B16 OVA₂₅₇₋₂₆₄ (B16 O)+1.25×10⁶ B16 GP₆₁₋₈₀ (B16 G) tumor cells (B16 O+G), or MCA OVA₂₅₇₋₂₆₄-GP₆₁₋₈₀ tumor cells were injected subcutaneously into mice. Antigen-specific T cells were adoptively transferred into tumor-bearing mice as described herein. For outgrowth experiments, tumors were measured manually with a caliper. Tumor volume was estimated with the formula (L×W×H)/2.

### Generation of Bone Marrow Chimeras and Depletion of Dendritic Cells In Vivo

B6 WT (CD45.1) mice were irradiated twice with 600 cGy, 6 hours apart. 12-18 hours later, bone marrow (BM) was isolated from femurs and tibias of CD11c-DTR/GFP (CD45.2) mice, and 5-8×10⁶ BM cells were injected i.v. into irradiated CD45.1 mice. BM chimeric were given antibiotics (trimethoprim-sulfamethoxazole) for 2 weeks. BM chimeric were analyzed for successful engraftment and BM reconstitution 6-8 weeks later. For conditional DC depletion, CD11c-DTR/GFP BM chimeric mice were injected (i.p.) with 4-5 ng/g body weight diphtheria toxin (DT, Sigma-Aldrich) every other day for 14 days.

### Generation of B16-I-A^{b}-Deficient Tumor Cell Line

The B16 tumor cells were subjected to CRISPR/Cas9-mediated knockout of I-A^{b} by transient transfection of a plasmid encoding both Cas9 nuclease and single guide (sg) RNA targeting the I-A^{b} locus, as well as GFP reporter gene. 2.5×10⁵ B16 cells were plated and transfected with 2 μg of Cas9- and sgRNA-encoding plasmid DNA using Lipofectamine 3,000 (Invitrogen) following the manufacturer's protocol. 3 days post transduction, GFP+ cells were FACS-sorted. Deletion of I-A^{b} was confirmed by treating GFP+ B16 I-A^{b} cells with 20 U/ml IFNγ for 48 h, followed by flow cytometric analysis of I-A^{b} expression.

### Color-Coded Tumor Model and Adoptive Transfer of Color-Coded T Cells

CD11c-YFP transgenic mice were injected subcutaneously with 2.5×10⁶ (1B16 (G) tumor cells or a mixture of 1.25×10⁶ B16-O+1.25×10⁶ B16-G tumor cells (B16 O+G). To generate color coded TCR_{OT1} CD8 T cells, TCR_{OT1} splenocytes were transduced to express tRFP using retroviral transduction as previously described [68]. Briefly, Platinum-E cells (ATCC) were transfected with a tRFP-encoding retroviral vector using the Mirus TransIT-LT1 reagent (catalog no. 2305). Viral supernatant was supplemented with polybrene and added to TCR_{OT1} splenocytes, and the cells were transduced via spinfection on two consecutive days. To generate color-coded TCR_{SMARTA} CD4 T cells, splenocytes from TCR_{SMARTA} GFP transgenic mice were used and activated as described above. Tumor-bearing mice were treated with cyclophosphamide (180 mg/kg) one day before ACT, and in vitro activated 2.5+10 TCR_{OT1} tRFP+ CD8 T cells and 4×10⁵ cells TCR_{SMARTA} EGFP CD4 T cells were transferred (i.v.) into tumor-bearing mice.

### Immunofluorescence Staining and Confocal Imaging

For confocal microscopy analysis, pieces of established tumors were excised and fixed for 18-24 hours in 4% paraformaldehyde solution, followed by dehydration in 20% sucrose, and then embedded in OCT, and stored at −80° C. 30-lam-thick frozen sections were cut on a CM3050S cryostat (Leica) and adhered to Superfrost Plus slides (Thermo Fisher Scientific). Nuclei were labeled using DAPI (Sigma). Slides were mounted with ProLong Diamond Antifade Mountant (Invitrogen) and analyzed on a Leica TCS SP8 confocal microscope. Fiji Is Just ImageJ (FIJI) was utilized for image analysis. 3D reconstitution was performed, and triple contacts/triads were assessed based on color-coded immune subset identification. Analyses was performed as a blinded outcome assessment. To quantify double contacts, after thresholding and binarization of images, the function “analyze particles” has been applied. To precisely estimate only events showing double contact, the mathematical function “AND” was used.

Isolation of Adoptively Transferred T Cells from Downstream Analyses

Lymph nodes were mechanically disrupted with the back of a 3-mL syringe, filtered through a 100-μm strainer, and red blood cells (RBC) were lysed with ammonium chloride potassium buffer. Cells were washed twice with cold RPMI 1640 media supplemented with 2 μM glutamine, 100 U/mL penicillin/streptomycin, and 3% fetal bovine serum (FBS). Tumor tissue was mechanically disrupted with a glass pestle and a 150-μm metal mesh in 5 mL of cold HBSS with 3% FBS. Cell suspension was filtered through 70-μm strainers. Tumor homogenate was spun down at 400 g for 5 minutes at 4° C. Pellet was resuspended in 15 mL. HBSS with 3% FBS, 500 μl (500 U) heparin, and 8 mL isotonic Percoll (GE), mixed by several inversions, and spun at 500 g for 10 min at 4° C. Pellet was lysed with ammonium chloride potassium buffer and cells were further processed for downstream applications.

### Sample Preparation for RNA-Seq and ATAC-Seq

TCR_{OT1} CD8 T cells were isolated from tumors (see above); cells were stained for CDc8α (clone 53-6.7, eBioscience) and CD45.1+(clone A20, Biolegend). CD8⁺CD45.1⁺ cells were sorted by FACS. For RNA-seq, T cells were directly sorted into Trizol LS reagent (Invitrogen, catalog no. 10296010) and stored at −80° C. For ATAC-seq, sorted T cells were resuspended in cold FBS with 10% DMSO and stored at −80° C.

### RNA-Seq

RNA from sorted cells was extracted using the RNeasy Mini Kit (Qiagen; catalog no. 74104) according to instructions provided by the manufacturer. After RiboGreen quantification and quality control by an Agilent BioAnalyzer, total RNA underwent amplification using the SMART-Seq v4 Ultra Low Input RNA Kit (Clontech), and amplified cDNA was used to prepare libraries with the KAPA Hyper Prep Kit (Kapa Biosystems). Samples were barcoded and run on a HiSeq 2500 in a 50-bp/50-bp paired-end run with the HiSeq SBS Kit v4 (Illumina). An average of 50 million paired reads were generated per sample.

### ATAC-Seq

Profiling of chromatin accessibility was performed by ATAC-seq as previously described (Buenrostro et al., 2013). Briefly, viably frozen, sorted T cells were washed in cold PBS and lysed. The transposition reaction was incubated at 42° C. for 45 min. The DNA was cleaned with the MinElute PCR Purification Kit (Qiagen; catalog no. 28004), and material was amplified for five cycles. After evaluation by real-time PCR, 7-13 additional PCR cycles were done. The final product was cleaned by AMPure XP beads (Beckman Coulter, catalog no. A63882) at a 1× ratio, and size selection was performed at a 0.5× ratio. Libraries were sequenced on a HiSeq 2500 or HiSeq 4000 in a 50-bp/50-bp paired-end run using the TruSeq SBS Kit v4, HiSeq Rapid SBS Kit v2, or HiSeq 3000/4000 SBS Kit (Illumina). An average of 100 million paired reads were generated per sample.

### Bioinformatics Methods

The quality of the sequenced reads was assessed with FastQC and QoRTs (for RNA-seq samples; Hartley and Mullikin, 2015; Andrews, 2010). Unless stated otherwise, plots involving high-throughput sequencing data were created using R version 4.1.0 (R Core Team, 2017) and ggplot2 (Wickham, 2016).

### RNA-Seq Data

DNA sequencing reads were aligned with default parameters to the mouse reference genome (GRCm38.p6) using STAR v2.6.0c (Dobin et al., 2013). Gene expression estimates were obtained with featureCounts v1.6.2 using composite gene models (union of the exons of all transcript isoforms per gene) from Gencode (version M17; Liao et al., 2014).

### DEGs

DEGs were determined using DESeq2 v1.34.0 with Wald tests with a q-value cutoff of 0.05 (Benjamini-Hochberg correction).

### Heatmaps

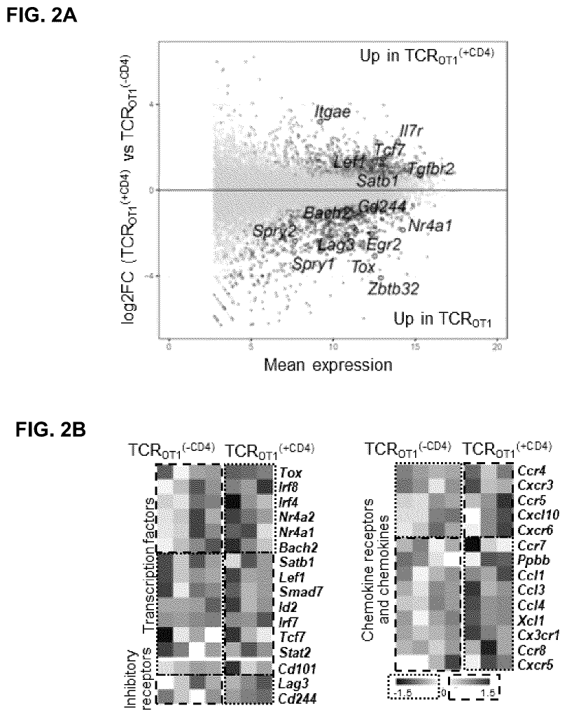

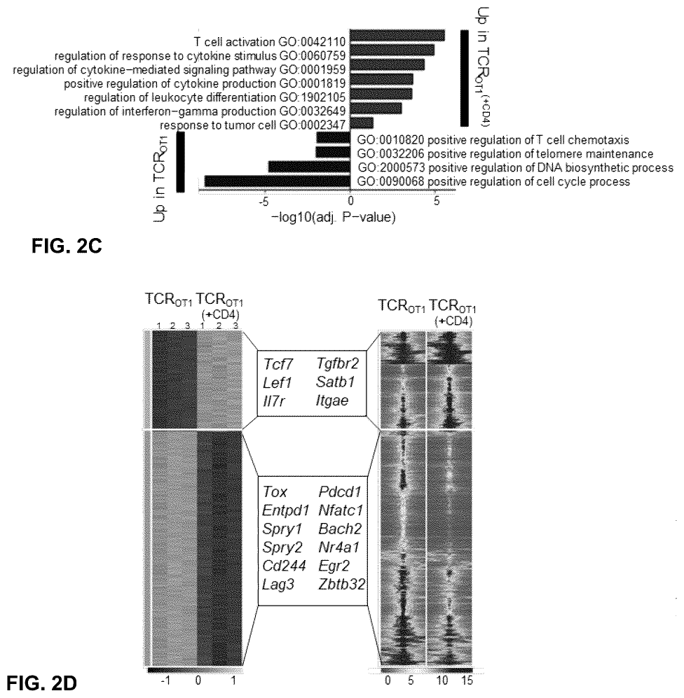

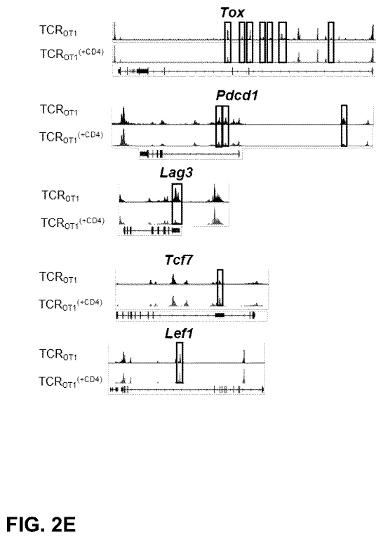

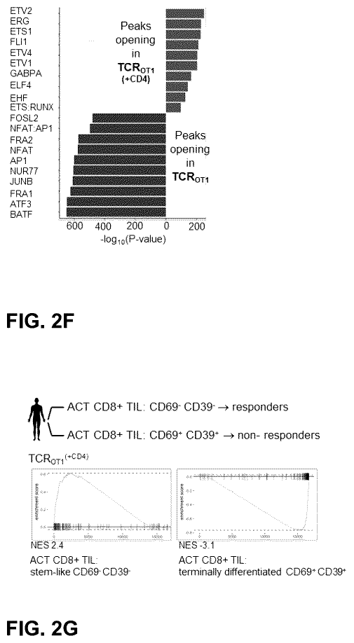

Heatmaps in FIG. 2B were created using DESeq2 normalized read counts after variance stabilizing transformation of genes identified as differentially expressed by DESeq2. Rows were centered and scaled.

### Pathway and GO Term Enrichment Analyses

Gene set enrichment analyses (FIG. 2G and FIG. 6) were done using fgsea 1.20.0 with the fgseaMultilevel function. Genes were ranked based on the DESeq2 Wald statistic. Gene sets with an FDR<0.05 were considered enriched.

Gene ontology analysis was performed on up- and down-regulated DEGs using the clusterProfiler v4.2.2 R package [70]. Only GO categories enriched using a 0.05 false discovery rate cutoff were considered.

### ATAC-Seq Data

Alignment and creation of peak atlas. Reads were aligned to the mouse reference genome (version GRCm38) with BWA-backtrack v0.7.17 (Li and Durbin, 2009). Post-alignment filtering was done with samtools v1.8 and Picard tools v2.18.9 (Li et al., 2009) to remove unmapped reads, improperly paired reads, nonunique reads, and duplicates. Peaks were called with MACS2 v2.1.1 (Liu, 2014), and peaks with adjusted P values smaller than 0.01 were excluded.

Consensus peak sets were generated for each condition if a peak was found in at least two replicates. Reproducible peaks from each condition were merged with DiffBlind v3.4.11 to create an atlas of accessible peaks, which was used for downstream analyses. The peak atlas was annotated using the ChIPseeker v1.30.3 [71] and TxDb.Mmusculus.UCSC.mm10.knownGene [Bioconductor Core Team and Bioconductor Package Maintainer (2019). TxDb.Mmusculus.UCSC.mm10.knownGene: Annotation package for TxDb object(s). R package version 3.10.0.]. Blacklisted regions were excluded (https://sites.google.com/site/anshulkundaje/projects/blacklists).

### Differentially Accessible Regions

Regions where the chromatin accessibility changed between different conditions were identified with DESeq2 v1.34.0, and only Benjamini-Hochberg corrected P values<0.05 were considered statistically significant.

### Coverage Files

Genome coverage files were normalized for differences in sequencing depth (RPGC normalization) with bamCoverage from deepTools v3.1.0. Replicates were averaged together using UCSC-tools bigWigMerge. Merged coverage files were used for display in Integrated Genomics Viewer shown in FIG. 2E.

### Heatmaps

Heatmaps based on the differentially accessible peaks identified between TCR_{OT1} and TCR^{(+CD4)} as shown in FIG. 2D were created using profileplyr v1.10.2 (T. Carroll and D. Barrows (2021). profileplyr: Visualization and annotation of read signal over genomic ranges with profileplyr. R package version 1.10.2.) and ComplexHeatmap v2.15.1 [72], by binning the region+/−1 kb around the peak summits in 20 bp bins. To improve visibility, bins with read counts greater than the 75th percentile+1.5*IQR were capped at that value.

### Motif Analyses

For identifying motifs enriched in differentially accessible peaks, we utilized HOMER via marge v0.0.4 ([73]; and [Robert A. Amezquita (2021). marge: API for HOMER in R for Genomic Analysis using Tidy Conventions. R package version 0.0.4.9999]). HOMER was run separately on hyper- or hypo-accessible peaks with the flags -size given and -mask. Motifs enriched in hyper- or hypo-accessible peaks were determined by comparing the rank differences (based on P value). The consensus peakset identified by DiffBind was used as the background set.

### Statistical Analyses

Statistical analyses on flow cytometric data were performed using unpaired two-tailed Student's t tests (Prism 7.0, GraphPad Software). A P value of 0.05 was considered statistically significant. All other statistical testing methods are described in figure legends.

### Example 2: Tumor-Specific CD4 T Cells Reverse Tumor-Specific CD8 T Cell Dysfunction/Exhaustion in Solid Tumors

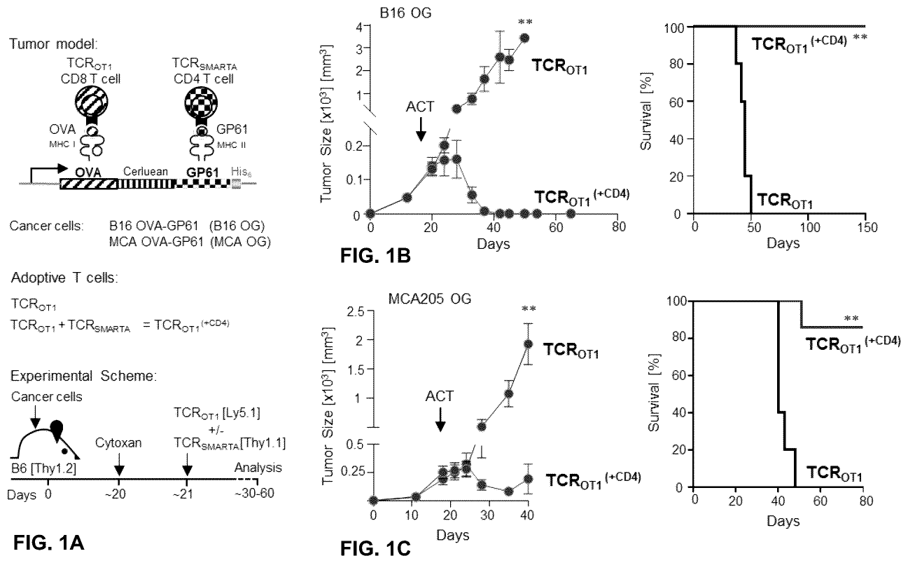

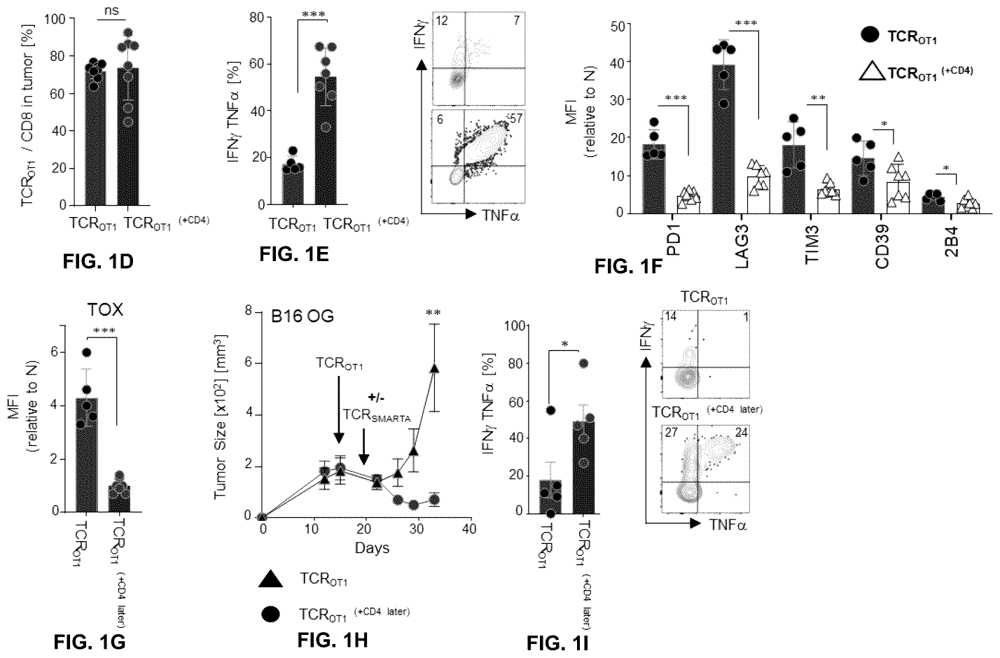

B16 is a highly aggressive murine melanoma cell line; B16 cancer cells injected subcutaneously into immunocompetent C57BL/6 wildtype mice (B6 WT) form large established tumors within 2 weeks, ultimately killing the host, and treatment regiments are generally ineffective. We engineered B16 cancer cells to express the CD8 T cell-recognized epitope from ovalbumin OVA₂₅₇₋₂₆₄ (SIINFEKL; SEQ ID NO: 2) as well as the CD4 T cell-recognized glycoprotein epitope GP₆₁₋₈₀ (GLKGPDIYKGVYQFKSVEFD; SEQ ID NO: 4) from the lymphocytic choriomeningitis virus (LCMV); the vector was constructed to encode the trimeric peptide sequence (SIINFEKLAAY)₃ (SEQ ID NO: 6) fused to the fluorescent protein Cerulean, followed by the 19-mer GP₆₁₋₈₀ peptide (FIG. 1A). The OVA₂₅₇₋₂₆₄ epitope is presented on the MHC class I molecule H-2K^{b} and recognized by TCR transgenic OT1 CD8 T cells (TCR_{OT1}); the GP₆₁₋₈₀ epitope is presented on the MHC class II I-A^{b} molecule and recognized by TCR transgenic SMARTA CD4 T cells (TCR_{SMARTA}). B16-OVA₂₅₇₋₂₆₄-GP₆₁₋₈₀ cancer cells (B16-OG; 2.5×10⁶ cells/host) were injected subcutaneously into B6 WT (CD45.2) mice. Despite the expression of strong CD8- and CD4-T cell tumor antigens, B16-OG tumors grew aggressively, forming large tumors within 2 weeks (FIG. 1B). We then employed an adoptive T cell transfer (ACT) regimen modeled on that used in cancer patients treated with ACT: preconditioning the host and inducing lymphopenia through a nonmyeloablative chemotherapeutic dose of cyclophosphamide followed by the infusion of in vitro generated cytotoxic tumor-specific CD8 T cells (FIG. 1A). Naïve congenic (CD45.1) TCR_{OT1} were activated in vitro for 3-4 days and adoptively transferred into lymphopenic B16-OG tumor-bearing mice. Despite the infusion of highly functional effector TCR_{OT1} CD8 T cells, B16-OG tumors progressed, recapitulating the scenario commonly observed in patients with solid tumors receiving ACT (FIG. 1B). Next, we asked whether the simultaneous infusion of in vitro activated effector TCR_{SMARTA} CD4 T cells would mediate anti-tumor responses. Co-transfer of effector TCR_{OT1} together with TCR_{SMARTA} resulted in complete tumor elimination, with 100% long-term tumor-free survival (FIG. 1B). Tumor-bearing mice that received TCR_{SMARTA} alone did not show tumor regression (data not shown), demonstrating that cancer elimination was dependent on both TCR_{OT1} and TCR_{SMARTA} T cells. We confirmed our results in a second tumor model using the fibrosarcoma cell line MCA205 (MCA205-OG) and obtained similar results (FIG. 1C).

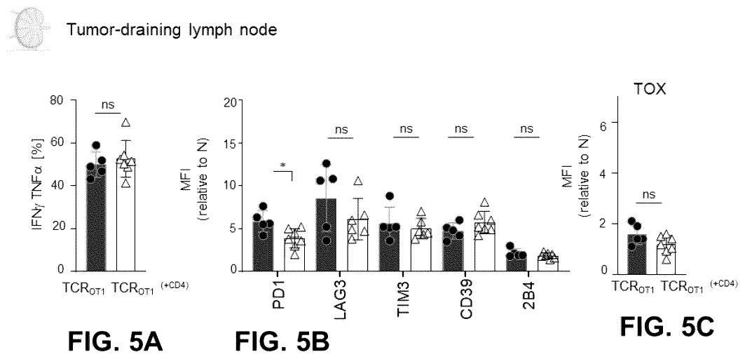

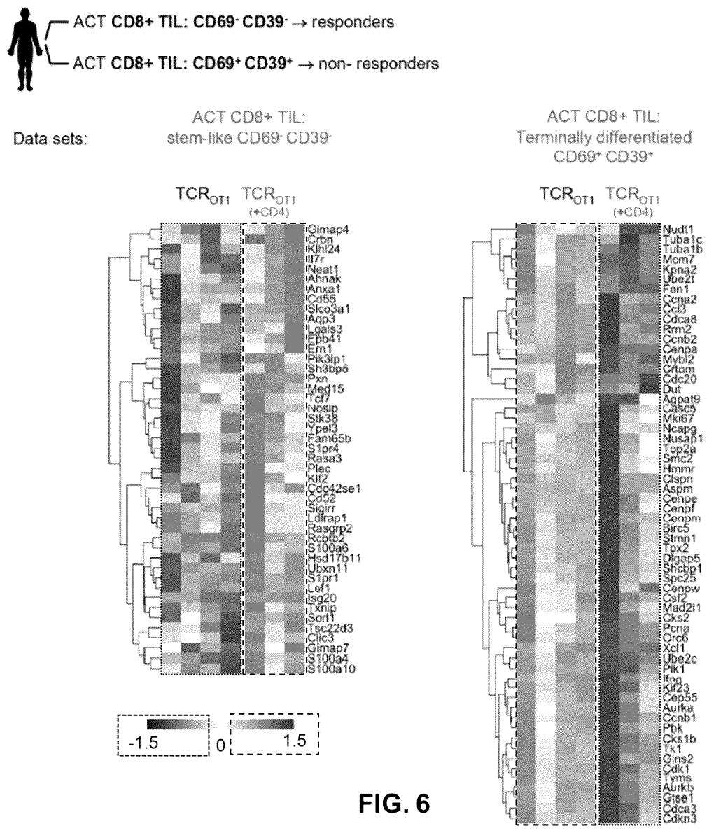

CD4 T cells are known to enhance CD8 T cell mobilization into peripheral tissues [28]. To understand whether TCR_{SMARTA} enhanced TCR_{OT1} tumor infiltration, we compared the numbers of TCR_{OT1} TIL in mice which received effector TCR_{OT1} alone (TCR_{OT1}) or together with TCR_{SMARTA} (TCR (+^{CD4})); we evaluated numbers of TIL 8-9 days post transfer, a time point when tumors are similar in size. Surprisingly, we found equal numbers of TCR_{OT1} TIL in both cohorts (FIG. 1D), suggesting that TCR_{SMARTA}-mediated anti-tumor immunity was not due to an enhancement of TCR_{OT1} tumor infiltration but likely due to functional changes of TCR_{OT1} TIL. Indeed, while TCR_{OT1} TIL were impaired in their ability to produce the effector cytokines IFNγ and TNFα (FIG. 1E), expressed high levels of numerous canonical inhibitory receptors including PD1, LAG3, TIM3, CD39 and 2B4 (FIG. 1F), as well as the transcription factor TOX (FIG. 1G), a critical regulator associated with T cell exhaustion [53-58], TCR^{(+CD4)} were able to produce high amounts of IFNγ and TNFα and showed little/no expression of inhibitory receptors and TOX (FIGS. 1E-1G). To understand whether these phenotypic and functional differences were already induced in the tumor-draining lymph node (tdLN), we compared phenotype and function of tdLN-TCR_{OT1} and tdLN-TCR^{(+CD4)} Interestingly, no differences were observed (FIG. 5), thus co-transferred CD4 T cells specifically acted on tumor-specific CD8 T cells within the tumor.

Next, we wanted to understand whether CD4 T cells could not only prevent but also reverse CD8 T cell dysfunction/exhaustion. We adoptively transferred effector TCR_{OT1} into B16-OG tumor-bearing mice, and 10 days later, when TCR_{OT1} TIL were dysfunctional/exhausted, we adoptively transferred effector TCR_{SMARTA}. Remarkably, mice that received TCR_{SMARTA} showed tumor regression while control cohorts did not (FIG. 111). Thus, tumor-reactive TCR_{SMARTA} CD4 T cells prevent and reverse tumor-induced CD8 T cell dysfunction and mediate tumor regression.

### Example 3: CD4 T Cells Transcriptionally and Epigenetically Reprogram Tumor-Specific CD8 T Cells, Leading to Tumor Elimination

Tumor-specific CD8 T cell dysfunction in mice and humans is associated with global transcriptional and epigenetic dysregulation of genes and pathways important for T cell differentiation and function. To understand how CD4 T cells mediated functional rescue of TCR_{OT1} CD8 T cells, we conducted RNA-seq and ATAC-seq of TCR^{(+CD4)} and TCR TIL isolated from size-matched B16-OG tumors 8 days post transfer. 1795 genes were differentially expressed (DEG) including exhaustion/dysfunction-associated TF and inhibitory receptors/activation markers (Tox, Irf4, Pdcd1 (PD1), Havcr2, Lag3, CD160, Cd244 (2B4)) (FIGS. 2A and 2B), which were highly expressed in TCR_{OT1}. In contrast, TF and molecules associated with stem-like progenitor T cell states were enriched and highly expressed in TCR^{(+CD4)} TIL, including genes encoding Tcf7 (TCF1), Il7r, Itgae (CD103), Itga1, and Ifitm3, as well as chemokine receptors such as Ccr5, Ccr4 and Ccr2 [30, 59]. Gene ontology (GO) classification revealed that pathways associated with positive cytokine regulation, immune differentiation and responses to tumor cells were enriched in TCR (+^{CD4}) but not in TCR_{OT1} (FIG. 2C). ATAC-seq revealed 11,787 differentially accessible regions (DAR), including enhancers in many exhaustion (Tox, Spry1 Spry2, Cd244, Bach2, Egr2) or stem-/progenitor cell state-associated genes (Tcf7, IL7r, Lef1), respectively (FIGS. 2D and 2E). Many enhancer peaks with TF motifs associated with terminal differentiation were less accessible in reprogrammed CD8 T cells, which was surprising given that TCR (+^{CD4}) and TCR TIL were isolated from equally sized tumors (FIG. 2F). To understand whether reprogrammed TCR (+^{CD4}) revealed molecular signatures similar to human CD8 TIL driving clinical responses in the context of ACT, we utilized a data set from a study conducted by the Rosenberg group, using ex vivo-expanded autologous CD8+ TIL from metastatic melanoma lesions for ACT into preconditioned, lymphodepleted patients [60]. The authors identified a CD39-CD69-stem-like TIL subset that was associated with complete cancer regression in ACT-responders but lacking in ACT-non-responders. Gene set enrichment analysis (GSEA) revealed that the same genes were enriched in TCR^{(+CD4)} CD8 TIL as in ACT (CD39-CD69−) CD8 TIL responders, and genes in CD8 TIL from ACT (CD39+CD69+) non-responders were enriched in TCR_{OT1} CD8 TIL (FIG. 2G, FIG. 6) [60].

Taken together, tumor-specific TCR_{SMARTA} CD4 T cells transcriptionally and epigenetically reprogram tumor-reactive CD8 TIL within progressing tumors, preventing terminal differentiation and exhaustion, and resulting in tumor elimination.

### Example 4: Spatial Positioning of Tumor-Specific CD8 and CD4 T Cells within Tumors Determine Anti-tumor Immunity and Cancer Elimination

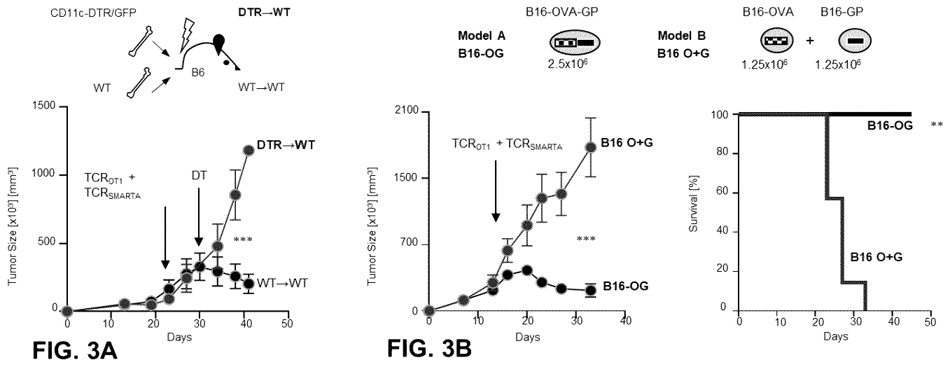

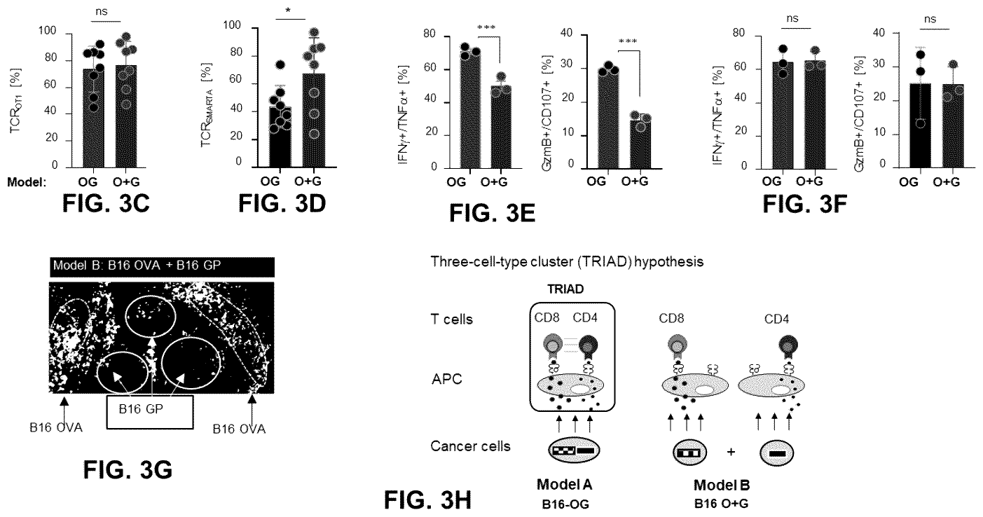

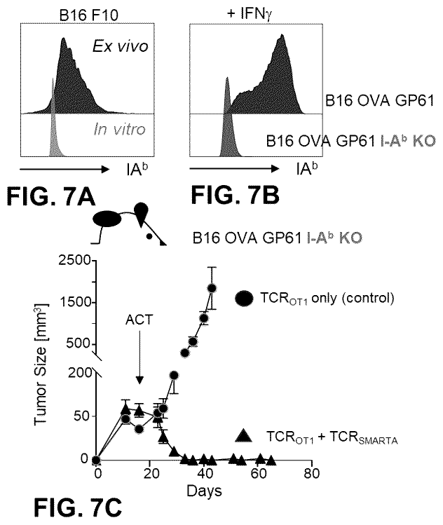

Next, we wanted to understand how TCR_{SMARTA} CD4 T cells prevent CD8 T cell exhaustion within tumors. B16 tumor cells express low level MHC II in vivo (FIG. 7A), thus cancer cells could become targets of CD4 T cells. Employing CRISPR/Cas9-mediated gene editing, we generated MHC class II I-Ab-deficient B16-OG cancer cells. Surprisingly, large established B16-OG I-Ab-deficient tumors were eliminated as efficiently as parental MHC class II-expressing B16-OG tumors, demonstrating that cancer elimination does not require CD4 T cell to directly target cancer cells (FIGS. 7B and 7C). Next, we turned to the tumor stroma, which includes MHC class I- and II-expressing antigen presenting cells (APC) such as CD11c+ dendritic cells (DC) and macrophages. To assess the role of CD11c+ cells, we employed a targeted depletion approach: CD11c+ DC from CD11c-DTR/GFP transgenic mice express the primate diphtheria toxin receptor (DTR) transgene under the CD11c promoter, enabling conditional depletion of CD11c+ cells in vivo upon DT treatment [61]. We generated bone marrow (BM) chimeras by transferring BM cells from CD11c-DTR/GFP (CD11c-DTR) or littermate control (WT) mice into lethally irradiated WT (CD45.1) B6 mice (designated “DTR→WT” and “WT→WT” chimeras). B16-OG tumors were established in DTR→WT and WT→WT BM chimeras, and 2-3 weeks post B16-OG tumor cell transplantation effector TCR_{OTI} and TCR_{SMARTA} were adoptively transferred. 5 days post ACT, when TCR_{OTI} and TCR_{SMARTA} infiltrated into tumors, mice were treated twice weekly with DT. Depletion of CD11c+ APC prevented tumor elimination in DTR→WT mice but not control WT→WT mice, suggesting that CD11c+ APC within the tumor microenvironment were necessary for TCR_{SMARTA}-mediated TCR_{OTI} reprogramming and tumor elimination (FIG. 3A).

Next, we wanted to investigate how TCR_{SMARTA}, TCR (+^{CD4}) and stromal cell interactions cause tumor elimination. To answer this question, we modified our tumor model (FIG. 3B): we generated B16 tumor cell lines expressing either the CD8-OVA (B16-0) or CD4-GP (B16-G) tumor antigens. We implanted a mixture of 1.25×10⁶ B16-0 and 1.25×10⁶ B16-G cancer cells into WT B6 mice, forming mixed B16 O+G tumors. Control mice received 2.5×10⁶ B16-OG tumor cells as in FIGS. 1 and 2; thus, both cohorts received the same total number (2.5×10⁶) of cancer cells, expressing similar levels of OVA and GP tumor antigens (data not shown). B16 O+G tumors grew with similar kinetics as B16-OG tumors. 2-3 weeks post tumor transplantation, mice received effector TCR_{OTI} and TCR_{SMARTA}. 7 days post ACT, equal numbers of TCR_{OTI} and TCR_{SMARTA} TIL were found within progressing B16 O+G and B16-OG tumors (FIGS. 3C, 3D). Strikingly, despite the same numbers of tumor cells, equal tumor sizes, and same numbers of TCR_{OT1} and TCR_{SMARTA} TIL, mixed B16 O+G tumors continued to grow, in contrast to B16-OG tumors, which ultimately regressed (FIG. 3B). TCR_{OT1} TIL isolated from B16 O+G tumors revealed a dysfunctional phenotype similar to those described for TCR_{OT1} transferred without CD4 T cells shown in FIG. 1 (FIG. 3E). Importantly, these functional differences were only observed within the tumor and not in the tdLN (FIG. 3F).

Without wishing to be bound by theory, it is believed that a unique spatial organization of cancer cells, CD4 T cells, CD8 T cells, and DC within tumors likely drove CD8 T cell reprogramming and tumor destruction.

### Example 5: Intratumoral Immune Triads in Mouse and Human Tumors are Required for Anti-Tumor Responses

To define the intratumoral spatial characteristics we conducted confocal microscopic analysis of established B16 O+G tumors. We found regions of either B16-OVA-positive and B16-GP-positive cancer cells, and very few regions that had B16-OVA and B16-GP cancer cells intermingled (FIG. 3G). The mosaic-like appearance of distinct tumor regions is a typical feature of clonally growing cancer cells in transplantation tumor models [45]. Consequently, in B16 O+G tumors CD8 or CD4 antigens are largely presented in distinct regions within the tumor and on distinct DC/APC (Model B), unlike in B16-OG tumors where CD8 and CD4 antigens are co-presented on the same DC/APC through epitope linkage (Model A) (FIG. 311). Thus, we propose the following model: co-presentation of tumor-specific CD4 and CD8 tumor antigens on the same APC will “force” antigen-specific CD4 and CD8 T cells to form three-cell-type clusters (triads) with APC, and the physical proximity of CD8 T cells with CD4 T cells drives CD4 T cell-mediated CD8 T cell reprogramming and cancer cell destruction (Model A). In Model B, CD8 and CD4 T cells fail to form triads with APC, CD4 T cells are unable to mediate CD8 T cell reprogramming, ultimately allowing tumors to progress. The concept of a ‘three-cell-type cluster’ was first described in 1987: Mitchison and O'Malley suggested that three-cell-type clusters of CD4 T cell-CD8 T cells-APC were required for the cytolytic response of CD8 T cells in an allogeneic transplant setting [62]. However, little is known about their functional relevance in vivo and/or underlying mechanisms.

To determine whether triads are indeed a requisite for tumor elimination, we generated color-coded B16 O+G and B16 O-G tumor models: TCR_{SMARTA} transgenic mice were crossed to EGFP transgenic mice, generating EGFP-expressing TCR_{SMARTA} CD4 T cells; TCR_{OT1} were engineered to express the red fluorescent protein (RFP); CD11c-YFP mice were used as hosts (with yellow fluorescent protein (YFP) under the transcriptional control of the CD11c promoter, thereby YFP-labeling CD11c+ host cells). B16-OG, B16-0, and B16-G cancer cells expressed Cerulean. B16-OG or B16 O+G tumors were established in CD11c-YFP mice and effector TCR_{OTI}-RFP+ and TCR_{SMARTA}-EGFP⁺ adoptively transferred (FIG. 4A). Strikingly, 8-9 days post ACT significantly higher numbers of TCR_{OT1}::CD11c⁺YFP⁺::TCR_{SMARTA} three-cell-clusters/triads (˜30 interactions/field (or close apposition)) were present in B16-OG tumors, which eventually regressed, in contrast to B16 O+G tumors (˜7 interactions), which eventually progressed (FIG. 4B). When normalized to the total number of infiltrating CD11c⁺YFP⁺ cells/field, which remained constant in both tumor models (FIG. 4C, right), we observed a 3.5-fold increase of triads in B16-OG tumors (FIG. 4C, left). Importantly, dyads, two-cell-interactions between TCR_{SMARTA}::CD11c⁺YFP⁺ DC, were not significantly different between B16-OG and B16 O+G (FIG. 4D). Thus, CD8 T cell::CD4 T cell::DC triads are associated with tumor-specific CD8 T cell reprogramming and tumor elimination.

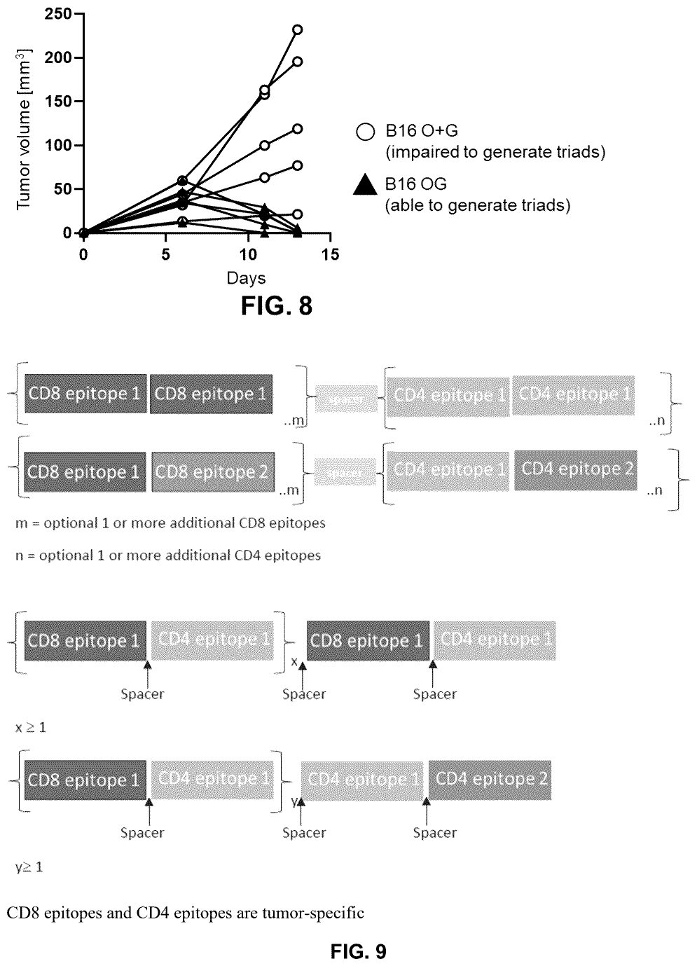

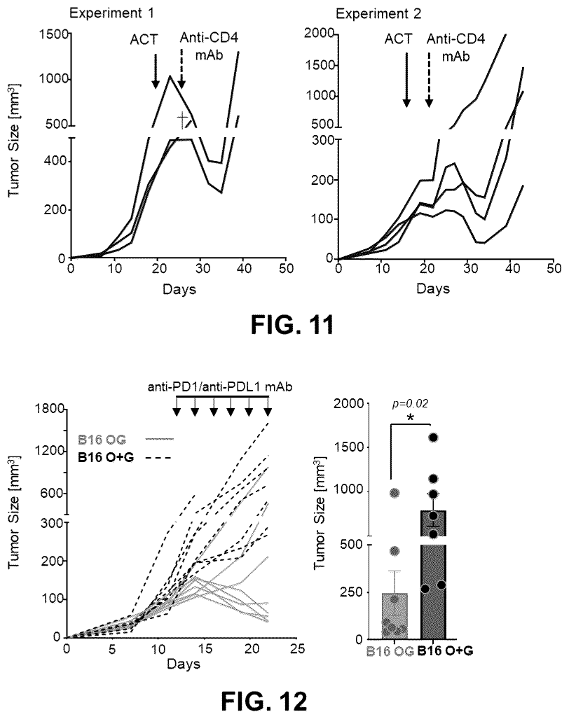

Triads were also required for immune-checkpoint blockade induced tumor elimination in a xenograft mouse model. See FIG. 8 and FIG. 12.

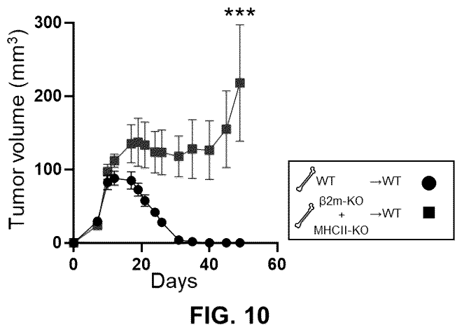

To demonstrate that CD4+ and CD8+ T cells must interact with the same antigen-presenting cell/dendritic cell (APC/DC) and form triads, we conducted a mixed bone marrow (BM) experiment: we generated BM chimeras from BM of beta-2-microglobulin-deficient (B2M KO) and MHC-II (I-Ab)-deficient (MHCII KO) donor mice or B6-control mice. We adoptively transferred CD8 TCR_{OT1} T cells and CD4 TCR_{SMARTA} T cells into B16-OG tumor-bearing BM chimeras and show that B16-OG tumors in mixed KO BM chimera mice do not get eliminated, conclusively demonstrating the requirement of CD4 and CD8 T cells to engage with the same APC/DC (FIG. 10).

When TCR_{SMARTA} CD4 T cells were co-transferred with TCR_{OT1} CD8 T cells into tumor-bearing B16-OG hosts and then depleted 5-6 days post-transfer through CD4+-depleting monoclonal antibody treatment, tumors initially regressed but eventually progressed, suggesting that continuous/sustained intratumoral triad formation and CD4+ T cells are required for CD8+ T cell-mediated tumor destruction (FIG. 11).

### Example 6: Triad Formation with Other CD4-Specific and CD8-Specific Epitopes

Additional studies are underway to investigate the role of triads in tumors which express different tumor antigens and CD8/CD4 T cell epitopes. We have generated tumor cell lines (B16, MCA205) which express the CD8 T cell epitope gp33, and CD4 T cell epitope ova-2. These epitopes are recognized by endogenous gp33-specific CD8 T cells and/or TCR transgenic P14 CD8 T cells, and endogenous ova-2 specific CD4 T cells and/or TCR transgenic OT-II CD4 T cells, respectively. Tumor cells will be implanted into B6 wild-type mice and once tumors are established, P14 and OT-II T cells will be adoptively transferred. It is anticipated that similar to the tumor model utilizing OVA and GP61, tumors that can generate triads will be eliminated by T cells, in contrast to tumors which are impaired in triad formation. It is also anticipated that immune checkpoint blockade (e.g. anti-PD1/anti-PDL1) will mediate tumor rejection in tumors that form triads in the tumor microenvironment.

These results will demonstrate that the triad formation observed with vaccines containing CD8-OVA (B16-0) and CD4-GP (B16-G) can be extrapolated to other tumor-specific epitopes recognized by CD4 and CD8 T cells.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.
1. Philip, M. and A. Schietinger, CD8(+) T cell differentiation and dysfunction in cancer. Nat Rev Immunol, 2022. 22(4): p. 209-223.
2. Anderson, K. G., I. M. Stromnes, and P. D. Greenberg, Obstacles Posed by the Tumor Microenvironment to T cell Activity: A Case for Synergistic Therapies. Cancer Cell, 2017. 31(3):p. 311-325.
3. Gajewski, T. F., H. Schreiber, and Y. X. Fu, Innate and adaptive immune cells in the tumor microenvironment. Nat Immunol, 2013. 14(10): p. 1014-22.
4. Yang, J. C. and S. A. Rosenberg, Adoptive T-Cell Therapy for Cancer. Adv Immunol, 2016. 130: p. 279-94.
5. Leko, V. and S. A. Rosenberg, Identifying and Targeting Human Tumor Antigens for T Cell-Based Immunotherapy of Solid Tumors. Cancer Cell, 2020. 38(4): p. 454-472.
6. Schmitt, T. M., et al., New Strategies in Engineering T-cell Receptor Gene-Modified T cells to More Effectively Target Malignancies. Clin Cancer Res, 2015. 21(23): p. 5191-7.
7. Rosenberg, S. A., et al., Gene transfer into humans—immunotherapy of patients with advanced melanoma, using tumor-infiltrating lymphocytes modified by retroviral gene transduction. N Engl J Med, 1990. 323(9): p. 570-8.
8. Schmitt, T. M., G. B. Ragnarsson, and P. D. Greenberg, T cell receptor gene therapy for cancer. Hum Gene Ther, 2009. 20(11): p. 1240-8.
9. Morgan, R. A., et al., Cancer regression in patients after transfer of genetically engineered lymphocytes. Science, 2006. 314(5796): p. 126-9.
10. Morgan, R. A., M. E. Dudley, and S. A. Rosenberg, Adoptive cell therapy: genetic modification to redirect effector cell specificity. Cancer J, 2010. 16(4): p. 336-41.
11. Rosenberg, S. A. and M. E. Dudley, Cancer regression inpatients with metastatic melanoma after the transfer of autologous antitumor lymphocytes. Proc Natl Acad Sci USA, 2004. 101 Suppl 2(Suppl 2): p. 14639-45.
12. Rosenberg, S. A., et al., Durable complete responses in heavily pretreated patients with metastatic melanoma using T-cell transfer immunotherapy. Clin Cancer Res, 2011. 17(13): p. 4550-7.
13. Yee, C., et al., Adoptive T cell therapy using antigen-specific CD8+ T cell clones for the treatment of patients with metastatic melanoma: in vivo persistence, migration, and antitumor effect of transferred T cells. Proc Natl Acad Sci USA, 2002. 99(25): p. 16168-73.
14. Chapuis, A. G., et al., T cell receptor gene therapy targeting WT1 prevents acute myeloid leukemia relapse post-transplant. Nat Med, 2019. 25(7): p. 1064-1072.
15. Dudley, M. E., et al., Cancer regression and autoimmunity inpatients after clonal repopulation with antitumor lymphocytes. Science, 2002. 298(5594): p. 850-4.
16. Stromnes, I. M., et al., T Cells Engineered against a Native Antigen Can Surmount Immunologic and Physical Barriers to Treat Pancreatic Ductal Adenocarcinoma. Cancer Cell, 2015. 28(5): p. 638-652.
17. Leen, A. M., C. M. Rooney, and A. E. Foster, Improving T cell therapy for cancer. Annu Rev Immunol, 2007. 25: p. 243-65.
18. Wrzesinski, C., et al., Increased intensity lymphodepletion enhances tumor treatment efficacy of adoptively transferred tumor-specific T cells. J Immunother, 2010. 33(1): p. 1-7.
19. Klebanoff, C. A., et al., Sinks, suppressors and antigen presenters: how lymphodepletion enhances T cell-mediated tumor immunotherapy. Trends Immunol, 2005. 26(2): p. 111-7.
20. Klebanoff, C. A., et al., IL-15 enhances the in vivo antitumor activity of tumor-reactive CD8+ T cells. Proc Natl Acad Sci USA, 2004. 101(7): p. 1969-74.
21. Restifo, N. P., M. E. Dudley, and S. A. Rosenberg, Adoptive immunotherapy for cancer: harnessing the T cell response. Nat Rev Immunol, 2012. 12(4): p. 269-81.
22. Baulu, E., et al., TCR-engineered T cell therapy in solid tumors: State of the art and perspectives. Sci Adv, 2023. 9(7): p. eadf3700.
23. Chandran, S. S. and C. A. Klebanoff, T cell receptor-based cancer immunotherapy: Emerging efficacy and pathways of resistance. Immunol Rev, 2019. 290(1): p. 127-147.
24. Borst, J., et al., CD4(+) T cell help in cancer immunology and immunotherapy. Nat Rev Immunol, 2018. 18(10): p. 635-647.
25. Bennett, S. R., et al., Help for cytotoxic-T-cell responses is mediated by CD40 signalling. Nature, 1998. 393(6684): p. 478-80.
26. Schoenberger, S. P., et al., T-cell help for cytotoxic T lymphocytes is mediated by CD40-CD40L interactions. Nature, 1998. 393(6684): p. 480-3.
27. Le Saout, C., et al., Memory-like CD8+ and CD4+ T cells cooperate to break peripheral tolerance under lymphopenic conditions. Proc Natl Acad Sci USA, 2008. 105(49): p. 19414-9.
28. Nakanishi, Y., et al., CD8(+) T lymphocyte mobilization to virus-infected tissue requires CD4(+) T-cell help. Nature, 2009. 462(7272): p. 510-3.
29. Bos, R. and L. A. Sherman, CD4+T-cell help in the tumor milieu is required for recruitment and cytolytic function of CD8+ T lymphocytes. Cancer Res, 2010. 70(21): p. 8368-77.
30. Castellino, F., et al., Chemokines enhance immunity by guiding naive CD8+ T cells to sites of CD4+ T cell-dendritic cell interaction. Nature, 2006. 440(7086): p. 890-5.
31. Castellino, F. and R. N. Germain, Cooperation between CD4+ and CD8+ T cells: when, where, and how. Annu Rev Immunol, 2006. 24: p. 519-40.
32. Greenberg, P. D., D. E. Kern, and M. A. Cheever, Therapy of disseminated murine leukemia with cyclophosphamide and immune Lyt-1-+,2− T cells. Tumor eradication does not require participation of cytotoxic T cells. J Exp Med, 1985. 161(5): p. 1122-34.
33. Frey, A. B., Rat mammary adenocarcinoma 13762 expressing IFN-gamma elicits antitumor CD4+ MHC class II-restricted T cells that are cytolytic in vitro and tumoricidal in vivo. J Immunol, 1995. 154(9): p. 4613-22.
34. Mumberg, D., et al., CD4(+) T cells eliminate MHC class II-negative cancer cells in vivo by indirect effects of IFN-gamma. Proc Natl Acad Sci USA, 1999. 96(15): p. 8633-8.
35. Monach, P. A., et al., A unique tumor antigen produced by a single amino acid substitution. Immunity, 1995. 2(1): p. 45-59.
36. Perez-Diez, A., et al., CD4 cells can be more efficient at tumor rejection than CD8 cells. Blood, 2007. 109(12): p. 5346-54.
37. Qin, Z. and T. Blankenstein, CD4+ T cell-mediated tumor rejection involves inhibition of angiogenesis that is dependent on IFN gamma receptor expression by nonhematopoietic cells. Immunity, 2000. 12(6): p. 677-86.
38. Egilmez, N. K., et al., Human CD4+ effector T cells mediate indirect interleukin-12-and interferon gamma-dependent suppression of autologous HLA-negative lung tumor xenografts in severe combined immunodeficient mice. Cancer Res, 2002. 62(9): p. 2611-7.
39. Broderick, L., et al., Human CD4+ effector memory T cells persisting in the microenvironment of lung cancer xenografts are activated by local delivery of IL-12 to proliferate, produce IFN-gamma, and eradicate tumor cells. J Immunol, 2005. 174(2): p. 898-906.
40. Muranski, P., et al., Tumor-specific Th17-polarized cells eradicate large established melanoma. Blood, 2008. 112(2): p. 362-73.
41. Greenberg, P. D., Adoptive T cell therapy of tumors: mechanisms operative in the recognition and elimination of tumor cells. Adv Immunol, 1991. 49: p. 281-355.
42. Hung, K., et al., The central role of CD4(+) T cells in the antitumor immune response. J Exp Med, 1998. 188(12): p. 2357-68.
43. Braumuller, H., et al., T-helper-1-cell cytokines drive cancer into senescence. Nature, 2013. 494(7437): p. 361-5.
44. Muller-Hermelink, N., et al., TNFR1 signaling and IFN-gamma signaling determine whether T cells induce tumor dormancy or promote multistage carcinogenesis. Cancer Cell, 2008. 13(6): p. 507-18.
45. Schietinger, A., et al., Bystander killing of cancer requires the cooperation of CD4(+) and CD8(+) T cells during the effector phase. J Exp Med, 2010. 207(11): p. 2469-77.
46. Espinosa-Carrasco, G., et al., CD4(+) T Helper Cells Play a Key Role in Maintaining Diabetogenic CD8(+) T Cell Function in the Pancreas. Front Immunol, 2017. 8: p. 2001.
47. Alspach, E., et al., MHC-II neoantigens shape tumour immunity and response to immunotherapy. Nature, 2019. 574(7780): p. 696-701.
48. Sahin, U., et al., Personalized RNA mutanome vaccines mobilize poly-specific therapeutic immunity against cancer. Nature, 2017. 547(7662): p. 222-226.
49. Ott, P. A., et al., An immunogenic personal neoantigen vaccine for patients with melanoma. Nature, 2017. 547(7662): p. 217-221.
50. Tran, E., et al., Cancer immunotherapy based on mutation-specific CD4+ T cells in a patient with epithelial cancer. Science, 2014. 344(6184): p. 641-5.
51. Hunder, N. N., et al., Treatment of metastatic melanoma with autologous CD4+ T cells against NYESO-1. N Engl J Med, 2008. 358(25): p. 2698-703.
52. Greenberg, P. D., M. A. Cheever, and A. Fefer, Eradication of disseminated murine leukemia by chemoimmunotherapy with cyclophosphamide and adoptively transferred immune syngeneic Lyt-1+2− lymphocytes. J Exp Med, 1981. 154(3): p. 952-63.
53. Scott, A. C., et al., TOX is a critical regulator of tumour-specific T cell differentiation. Nature, 2019. 571(7764): p. 270-274.
54. Kim, K., et al., Single-cell transcriptome analysis reveals TOX as a promoting factor for T cell exhaustion and a predictor for anti-PD-1 responses in human cancer. Genome Med, 2020. 12(1): p. 22.
55. Seo, H., et al., TOX and TOX2 transcription factors cooperate with NR4A transcription factors to impose CD8(+) T cell exhaustion. Proc Natl Acad Sci USA, 2019. 116(25): p. 12410-12415.
56. Wang, X., et al., TOX promotes the exhaustion of antitumor CD8(+) T cells by preventing PD1 degradation in hepatocellular carcinoma. J Hepatol, 2019. 71(4): p. 731-741.
57. Khan, O., et al., TOX transcriptionally and epigenetically programs CD8(+) T cell exhaustion. Nature, 2019. 571(7764): p. 211-218.
58. Alfei, F., et al., TOX reinforces the phenotype and longevity of exhausted T cells in chronic viral infection. Nature, 2019. 571(7764): p. 265-269.
59. Wu, M., H. Fang, and S. T. Hwang, Cutting edge: CCR4 mediates antigen-primed T cell binding to activated dendritic cells. J Immunol, 2001. 167(9): p. 4791-5.
60. Krishna, S., et al., Stem-like CD8 T cells mediate response of adoptive cell immunotherapy against human cancer. Science, 2020. 370(6522): p. 1328-1334.
61. Jung, S., et al., In vivo depletion of CD11c+ dendritic cells abrogates priming of CD8+ T cells by exogenous cell-associated antigens. Immunity, 2002. 17(2): p. 211-20.
62. Mitchison, N. A. and C. O'Malley, Three-cell-type clusters of T cells with antigen-presenting cells best explain the epitope linkage and noncognate requirements of the in vivo cytolytic response. Eur J Immunol, 1987. 17(11): p. 1579-83.
63. Lee, H. S., et al., A Phase II Window of Opportunity Study of Neoadjuvant PD-L1 versus PD-L1 plus CTLA-4 Blockade for Patients with Malignant Pleural Mesothelioma. Clin Cancer Res, 2023. 29(3): p. 548-559.
64. Keene, J. A. and J. Forman, Helper activity is required for the in vivo generation of cytotoxic T lymphocytes. J Exp Med, 1982. 155(3): p. 768-82.
65. Hu, H. M., et al., Divergent roles for CD4+ T cells in the priming and effector memory phases of adoptive immunotherapy. J Immunol, 2000. 165(8): p. 4246-53.
66. Gao, F. G., et al., Antigen-specific CD4+ T-cell help is required to activate a memory CD8+ T cell to a fully functional tumor killer cell. Cancer Res, 2002. 62(22): p. 6438-41.
67. Magen, A., et al., Intratumoral dendritic cell-CD4(+) T helper cell niches enable CD8(+) T cell differentiation following PD-1 blockade in hepatocellular carcinoma. Nat Med, 2023. 29(6): p. 1389-1399.
68. Shakiba, M., et al., TCR signal strength defines distinct mechanisms of T cell dysfunction and cancer evasion. J Exp Med, 2022. 219(2).
69. Korotkevich, G., et al., Fast gene set enrichment analysis. bioRxiv, 2021: p. 060012.
70. Wu, T., et al., clusterProfiler 4.0: A universal enrichment tool for interpreting omics data. Innovation (Camb), 2021. 2(3): p. 100141.
71. Yu, G., L. G. Wang, and Q. Y. He, ChIPseeker: an R/Bioconductor package for ChIP peak annotation, comparison and visualization. Bioinformatics, 2015. 31(14): p. 2382-3.
72. Gu, Z., R. Eils, and M. Schlesner, Complex heatmaps reveal patterns and correlations in multidimensional genomic data. Bioinformatics, 2016. 32(18): p. 2847-9.
73. Heinz, S., et al., Simple combinations of lineage-determining transcription factors prime cis-regulatory elements required for macrophage and B cell identities. Mol Cell, 2010. 38(4): p. 576-89.

What is claimed is:

1. A vaccine including
a polypeptide comprising (i) an oligomer of at least one tumor-specific peptide epitope recognized by CD8 T cells and (ii) an oligomer of at least one tumor-specific peptide epitope recognized by CD4 T cells,
wherein the oligomer of the at least one tumor-specific peptide epitope recognized by CD8 T cells is linked to the oligomer of the at least one tumor-specific peptide epitope recognized by CD4 T cells via a linker, and wherein the oligomer of the at least one tumor-specific peptide epitope recognized by CD4 T cells is at least a dimer, or
a nucleic acid molecule encoding the polypeptide.

2. The vaccine of claim 1, wherein the oligomer of the at least one tumor-specific peptide epitope recognized by CD8 T cells is a dimer, a trimer, a tetramer, a pentamer, a hexamer, a heptamer, an octamer, a nonamer, a decamer, an 11-mer, a 12-mer, a 13-mer, a 14-mer or a 15-mer; or
wherein the oligomer of the at least one tumor-specific peptide epitope recognized by CD4 T cells is a dimer, trimer, a tetramer, a pentamer, a hexamer, a heptamer, an octamer, a nonamer, a decamer, an 11-mer, a 12-mer, a 13-mer, a 14-mer or a 15-mer; or
wherein the oligomer of the at least one tumor-specific peptide epitope recognized by CD8 T cells comprises a single tumor-specific peptide epitope or 2-15 distinct tumor-specific peptide epitopes; or
wherein the oligomer of the at least one tumor-specific peptide epitope recognized by CD4 T cells comprises a single tumor-specific peptide epitope or 2-15 distinct tumor-specific peptide epitopes.

3. A vaccine comprising at least two tandem repeats of a polypeptide, wherein the polypeptide comprises (i) at least one tumor-specific peptide epitope recognized by CD8 T cells, and (ii) at least one tumor-specific peptide epitope recognized by CD4 T cells,
wherein the at least one tumor-specific peptide epitope recognized by CD8 T cells is linked to the at least one tumor-specific peptide epitope recognized by CD4 T cells via a linker, or
a nucleic acid molecule encoding the at least two tandem repeats of the polypeptide.

4. The vaccine of claim 3, comprising at least 15 tandem repeats of the polypeptide; or
wherein the at least one tumor-specific peptide epitope recognized by CD4 T cells is a monomer or an oligomer; or
wherein the at least one tumor-specific peptide epitope recognized by CD8 T cells is a monomer or an oligomer; or
wherein each tandem repeat of the polypeptide comprises the same tumor-specific peptide epitope recognized by CD8 T cells; or
wherein at least one of the at least two tandem repeats of the polypeptide comprise distinct tumor-specific peptide epitopes recognized by CD8 T cells; or
wherein each tandem repeat of the polypeptide comprises the same tumor-specific peptide epitope recognized by CD4 T cells; or
wherein at least one of the at least two tandem repeats of the polypeptide comprise distinct tumor-specific peptide epitopes recognized by CD4 T cells; or
wherein the nucleic acid molecule comprises RNA or DNA.

5. The vaccine of claim 1, wherein the linker is a cleavable peptide linker or a rigid peptide linker; or
wherein the linker comprises a GS₄ linker, a GS₃ linker, a P2A linker, a T2A linker, an E2A linker, a F2A linker, a BmCPV2A linker, an AAY linker, a GPGPG linker, an EAAAK linker, a HEYGAEALERAG linker, a KK linker, or an RVRR linker; or
wherein the linker comprises an ubiquitination sequence; or
wherein the at least one tumor-specific peptide epitope recognized by CD8 T cells and/or the at least one tumor-specific peptide epitope recognized by CD4 T cells is a tumor antigen; or
wherein the at least one tumor-specific peptide epitope recognized by CD8 T cells and/or the at least one tumor-specific peptide epitope recognized by CD4 T cells is a personalized neoantigen specific for a cancer subject.

6. The vaccine of claim 1, wherein the at least one tumor-specific peptide epitope recognized by CD8 T cells is derived from one or more tumor antigens selected from among MAGE, BAGE, GAGE, NY-ESO-1, Tyrosinase, Melan-A, gp100, CEA, MART-1, HER2, WT1, MUC1, ppCT, Beta-catenin, CDK4, LPGAT1, CASP-8, CDKN2A, HLA-A11d, CLPP, GPNMB, RBAF600, SIRT2, SNRPD1, SNRP116, MART2, MUM-1f, MUM-2, MUM-3, Myosin class I, N-ras, OS-9, Elongation factor 2, NFYC, Alpha-actinin-4, Malic enzyme, HLA-A2, Hsp70-2, SETDB1, METTL17, ALDH1A1, CDKN2A, TKT, SEC24A, EXOC8, MRPS5, PABPC1, KIF2C, POLA2, CCT6A, TRRAP, DNMT1, PABPC3, MAGE-A10, FMN2, TMEM48, AKAP13, OR8B3, WASL, MAGEA6, PDS5A, MED13, FLNA, KIB1B, KFI1BP, NARFL, PPFIA4, CDC37L1, MLL3, FLNA, DOPEY2, TTBK2, KIF26B, SPOP, RETSAT, CLINT1, COX7A2, FAM3C, CSMD1, PPP1R3B, CDK12, CSNK1A1, GAS7, MATN, HAUS3, MTFR2, CHTF18, MYADM, HERC1 and HSDL1; or
wherein the at least one tumor-specific peptide epitope recognized by CD8 T cells is selected from among: AEPINIQTW (SEQ ID NO: 7), FPSDSWCYF (SEQ ID NO: 8), SYLDSGIHF (SEQ ID NO: 9), ACDPHSGHFV (SEQ ID NO: 10), AVCPWTWLRG (SEQ ID NO: 11), ILDKVLVHL (SEQ ID NO: 12), TLDWLLQTPK (SEQ ID NO: 13), RPHVPESAF (SEQ ID NO: 14), KIFSEVTLK (SEQ ID NO: 15), SHETVIIEL (SEQ ID NO: 16), KILDAVVAQK (SEQ ID NO: 17), FLEGNEVGKTY (SEQ ID NO: 18), EEKLIVVLF (SEQ ID NO: 19), SELFRSGLDSY (SEQ ID NO: 20), FRSGLDSYV (SEQ ID NO: 21), EAFIQPITR (SEQ ID NO: 22), KINKNPKYK (SEQ ID NO: 23), ILDTAGREEY (SEQ ID NO: 24), KELEGILLL (SEQ ID NO: 25), ETVSEQSNV (SEQ ID NO: 26), QQITKTEV (SEQ ID NO: 27), FIASNGVKLV (SEQ ID NO: 28), FLDEFMEGV (SEQ ID NO: 29), SLFEGIDIYT (SEQ ID NO: 30), VESEDIAEL (SEQ ID NO: 31), RTKVVQTLW (SEQ ID NO: 32), IPIDGIFFT (SEQ ID NO: 33), KMIGNHLWV (SEQ ID NO: 34), AMFWSVPTV (SEQ ID NO: 35), CLNEYHLFL (SEQ ID NO: 36), KLMNIQQKL (SEQ ID NO: 37), QLSCISTYV (SEQ ID NO: 38), FLYNLLTRV (SEQ ID NO: 39), IILVAVPHV (SEQ ID NO: 40), HLYASLSRA (SEQ ID NO: 41), MLGEQLFPL (SEQ ID NO: 42), RLFPGLTIKI (SEQ ID NO: 43), TRSSGSHFVF (SEQ ID NO: 44), LRTKVYAEL (SEQ ID NO: 45), LLYQELLPL (SEQ ID NO: 46), IYKAPCENW (SEQ ID NO: 47), YYPPSQIAQL (SEQ ID NO: 48), LYNGMEHLI (SEQ ID NO: 49), HSVSSAFKK (SEQ ID NO: 50), YPPPPPALL (SEQ ID NO: 51), KVDPIGHVY (SEQ ID NO: 52), LMKVDPIGHVY (SEQ ID NO: 53), KVDPIGHVYF (SEQ ID NO: 54), FVVPYMIYLL (SEQ ID NO: 55), VSVQIISCQY (SEQ ID NO: 56), VQIISCQY (SEQ ID NO: 57), CVRVSGQGL (SEQ ID NO: 58), APARLERRHSA (SEQ ID NO: 59), AYHSIEWAI (SEQ ID NO: 60), YHSIEWAI (SEQ ID NO: 61), NAYHSIEWAI (SEQ ID NO: 62), KSQREFVRR (SEQ ID NO: 63), MRMNQGVCC (SEQ ID NO: 64), FLSDHLYLV (SEQ ID NO: 65), KPSDTPRPVM (SEQ ID NO: 66), HIAKSLFEV (SEQ ID NO: 67), AGQHIAKSLF (SEQ ID NO: 68), KPFCVLISL (SEQ ID NO: 69), RPHHDQRSL (SEQ ID NO: 70), SSYTGFANK (SEQ ID NO: 71), FLLDEAIGL (SEQ ID NO: 72), ALDPHSGHFV (SEQ ID NO: 73), HSCVMASLR (SEQ ID NO: 74), HDLGRLHSC (SEQ ID NO: 75), VSKILPSTW (SEQ ID NO: 76), GVADVLLYR (SEQ ID NO: 77), TESPFEQHI (SEQ ID NO: 78), GLEREGFTF (SEQ ID NO: 79), YTDFHCQYV (SEQ ID NO: 80), CILGKLFTK (SEQ ID NO: 81), GLFGDIYLA (SEQ ID NO: 82), SLADEAEVYL (SEQ ID NO: 83), KTLTSVFQK (SEQ ID NO: 84), ILNAMIAKIJ (SEQ ID NO: 85), FAFQEYDSF (SEQ ID NO: 86), LLDIVAPK (SEQ ID NO: 87), SPMIVGSPW (SEQ ID NO: 88), ASNASSAAK (SEQ ID NO: 89), CYMEAVAL (SEQ ID NO: 90), SIY, SVGDFSQEF (SEQ ID NO: 100), VSVGDFSQEF (SEQ ID NO: 101), KLKFVTLVF (SEQ ID NO: 102), VLAKKLKFV (SEQ ID NO: 103), FLFQDSKKI (SEQ ID NO: 104), NSKKKWFLF (SEQ ID NO: 105), VQKVASKIPF (SEQ ID NO: 106), ALFASRPRF (SEQ ID NO: 107), RFLEYLPLRF (SEQ ID NO: 108), TELERFLEY (SEQ ID NO: 109), LLHTELERF (SEQ ID NO: 110), LLHTELERFL (SEQ ID NO: 111), TLFHTFYEL (SEQ ID NO: 112), TLFHTFYELL (SEQ ID NO: 113), LFHTFYELLI (SEQ ID NO: 114), LFHTFYELL (SEQ ID NO: 115), TTLFHTFYEL (SEQ ID NO: 116), KFGDLTNNF (SEQ ID NO: 117), KLFESKAEL (SEQ ID NO: 118), KLFESKAELA (SEQ ID NO: 119), YNSFSSAPM (SEQ ID NO: 120), SFSSAPMPQI (SEQ ID NO: 121), GIPENSFNV (SEQ ID NO: 122), SVGDFSQEF (SEQ ID NO: 123), VSVGDFSQEF (SEQ ID NO: 124), TPAAPTAMA (SEQ ID NO: 125), FPGNQWNPV (SEQ ID NO: 126), LADFRLARLY (SEQ ID NO: 127), NHDETSFLL (SEQ ID NO: 128), TPAHPSQGA (SEQ ID NO: 129), TPAHPSQGAV (SEQ ID NO: 130), VTEKLQPTY (SEQ ID NO: 131), HPAPPAPPPA (SEQ ID NO: 132), VPKEHPAPPA (SEQ ID NO: 133), RPAARGSRV (SEQ ID NO: 134), FPKKIQMLA (SEQ ID NO: 135), FLDREQRESY (SEQ ID NO: 136), FPAAAFPTA (SEQ ID NO: 137), SPVTFPAAA (SEQ ID NO: 138), FPAAAFPTAS (SEQ ID NO: 139), ITDAHELGV (SEQ ID NO: 140), ITDAHELGVA (SEQ ID NO: 141), YTWPSGNIY (SEQ ID NO: 142), VLSSLVLVPL (SEQ ID NO: 143), NVLSSLVLV (SEQ ID NO: 144), SLPSNVLSSL (SEQ ID NO: 145), KIIAYQPYGK (SEQ ID NO: 146), RLMLRKVALK (SEQ ID NO: 147), QRLMLRKVAL (SEQ ID NO: 148), RLMLRKVAL (SEQ ID NO: 149), SLQRLMLRKV (SEQ ID NO: 150), ALQSQSISLV (SEQ ID NO: 151), ALQSQSISL (SEQ ID NO: 152), YLLFQNTDL (SEQ ID NO: 153), ALSPDGSIRK (SEQ ID NO: 154), FLLTDYALS (SEQ ID NO: 155), LLFAPEYGPK (SEQ ID NO: 156), WRNILLLSLH (SEQ ID NO: 157), SLHKGSLYPR (SEQ ID NO: 158), TLLSQVNKV (SEQ ID NO: 159), VRTLLSQVNK (SEQ ID NO: 160), RTAPRPGSQK (SEQ ID NO: 161), SLLRAAFFGK (SEQ ID NO: 162), LRAAFFGKCF (SEQ ID NO: 163), LRFNLIANQH (SEQ ID NO: 164), KLNFRLFVI (SEQ ID NO: 165), RKLNFRLFVI (SEQ ID NO: 166), KLNFRLFVIR (SEQ ID NO: 167), RIYTGEKPFK (SEQ ID NO: 168), FEAEFTQVA (SEQ ID NO: 169), ILMHGLVSL (SEQ ID NO: 170), RRNDDKSILM (SEQ ID NO: 171), SILMHGLVSL (SEQ ID NO: 172), RRWSALVIGL (SEQ ID NO: 173), KRRWSALVI (SEQ ID NO: 174), RRWSALVIG (SEQ ID NO: 175), STTKRRWSAL (SEQ ID NO: 176), YMASEVVEV (SEQ ID NO: 177), YMASEVVEVF (SEQ ID NO: 178), DEQIESMTY (SEQ ID NO: 179), KQAKVVNPPI (SEQ ID NO: 180), FMMPRIVDV (SEQ ID NO: 181), FMMPRIVDVT (SEQ ID NO: 182), MTFSSTKDYV (SEQ ID NO: 183), NEVSEVTVF (SEQ ID NO: 184), SQFARVPGYV (SEQ ID NO: 185), YVGSPLAAM (SEQ ID NO: 186), SQFARVPGY (SEQ ID NO: 187), WLVDLLPST (SEQ ID NO: 188), EEFWLVDLL (SEQ ID NO: 189), EEFWLVDLLP (SEQ ID NO: 190), HLYAYHEEL (SEQ ID NO: 191), EELSATVPS (SEQ ID NO: 192), EELSATVPSQ (SEQ ID NO: 193), FVADWAGTF (SEQ ID NO: 194), EESGGAVAFF (SEQ ID NO: 195), ESGGAVAFF (SEQ ID NO: 196), IPLSDNTIF (SEQ ID NO: 197), REFDKIELA (SEQ ID NO: 198), REFDKIELAY (SEQ ID NO: 199), SYQRYSHPLF (SEQ ID NO: 200), IYLHGSTDKL (SEQ ID NO: 201), YPVIFKSIM (SEQ ID NO: 202), KEYPVIFKSI (SEQ ID NO: 203), EYPVIFKSI (SEQ ID NO: 204), IFKSIMRQRL (SEQ ID NO: 205), YDYVSALHPV (SEQ ID NO: 206), HTHYDYVSAL (SEQ ID NO: 207), DYVSALHPV (SEQ ID NO: 208), FPQGLPNEY (SEQ ID NO: 209), LPNEYAFVTT (SEQ ID NO: 210), LPNEYAFVT (SEQ ID NO: 211), NEYAFVTTF (SEQ ID NO: 212), QGLPNEYAF (SEQ ID NO: 213), ALPQSILLF (SEQ ID NO: 214), TALPQSILLF (SEQ ID NO: 215), SPVLRSHSF (SEQ ID NO: 216), SYQLYTHPL (SEQ ID NO: 217), RFANPRDSF (SEQ ID NO: 218), TIIDNIKEM (SEQ ID NO: 219), LFSPGAANLF (SEQ ID NO: 220), FSPGAANLF (SEQ ID NO: 221), QPPALSPSY (SEQ ID NO: 222), APSPGQPPAL (SEQ ID NO: 223), FPSQRTSWEF (SEQ ID NO: 224), MPFTTVSELM (SEQ ID NO: 225), SELMKVSAM (SEQ ID NO: 226), HQFHVHPLL (SEQ ID NO: 227), MTITSRGTTV (SEQ ID NO: 228), RYGIRGFSTI (SEQ ID NO: 229), YGIRGFSTI (SEQ ID NO: 230), MAGPKGFQY (SEQ ID NO: 231), DMKARQKAL (SEQ ID NO: 232), DMKARQKALV (SEQ ID NO: 233), TRKNKKLAL (SEQ ID NO: 234), QTRKNKKLAL (SEQ ID NO: 235), LFRIKFKEPL (SEQ ID NO: 236), ISDRFIGIY (SEQ ID NO: 237), EISDRFIGIY (SEQ ID NO: 238), EISDRFIGI (SEQ ID NO: 239), QTSIQSPSLY (SEQ ID NO: 240), TSIQSPSLY (SEQ ID NO: 241), EMKRVFGFPV (SEQ ID NO: 242), VVDFKKNLEY (SEQ ID NO: 243), AAQARLQPV (SEQ ID NO: 244), HLARHRHLM (SEQ ID NO: 245), SPHLARHRHL (SEQ ID NO: 246), LLDKFVEWY (SEQ ID NO: 247), MPAWRTRGAI (SEQ ID NO: 248), MPAWRTRGA (SEQ ID NO: 249), LPVTRKNMPL (SEQ ID NO: 250), WTNCILHEY (SEQ ID NO: 251), HTLGAASSFM (SEQ ID NO: 252), HTLGAASSF (SEQ ID NO: 253), QLFARARPM (SEQ ID NO: 254), ISSQPQVPFY (SEQ ID NO: 255), SSQPQVPFY (SEQ ID NO: 256), NVELRRNVL (SEQ ID NO: 257), ESDLNSWPV (SEQ ID NO: 258), LPSFRPPTAL (SEQ ID NO: 259), ISIQRAQPL (SEQ ID NO: 260), ESIKEITNFK (SEQ ID NO: 261), SIKEITNFK (SEQ ID NO: 262), and ESIKEITNF (SEQ ID NO: 263); or
wherein the at least one tumor-specific peptide epitope recognized by CD4 T cells is derived from one or more tumor antigens selected from among COA-1, ARTC1, CDC27, FN1, LDLR-FUT fusion protein, neo-PAP, PTPRK and Triosephosphate isomerase; or
wherein the at least one tumor-specific peptide epitope recognized by CD4 T cells is selected from among: TLYQDDTLTLQAAGE (SEQ ID NO: 91), YSVYFNLPADTIYTNH (SEQ ID NO: 92), FSWAMDLDPKGAE (SEQ ID NO: 93), MIFEKHGFRRTTPP (SEQ ID NO: 94), WRRAPAPGA (SEQ ID NO: 95), PVTWRRAPA (SEQ ID NO: 96), RVIKNSIRLTLE (SEQ ID NO: 97), PYYFAAELPPRNLPEP (SEQ ID NO: 98), GELIGILNAAKVPAD (SEQ ID NO: 99), HEL, DRSVLAKKLKFVTLVFRHGDRSPID (SEQ ID NO: 264), NNSKKKWFLFQDSKKIQVEQPQ (SEQ ID NO: 265), SPIKLVQKVASKIPFPDRITEESV (SEQ ID NO: 266), TKRQVILLHTELERFLEYLPLRF (SEQ ID NO: 267), SHTQTTLFHTFYELLIQKNKHK (SEQ ID NO: 268), RLVLGKFGDLTNNFSSPHAR (SEQ ID NO: 269), LSPREEFLRLCKKIMMRSIQ (SEQ ID NO: 270), PSTANYNSFSSAPMPQIPVASVTPT (SEQ ID NO: 271), LCPREEFLRLCKKIMMRSIQ (SEQ ID NO: 272), SHNELADSGIPENSFNVSSLVE (SEQ ID NO: 273), SGSPPLRVSVGDFSQEFSPIQEAQQD (SEQ ID NO: 274), RPAGRTQLLWTPAAPTAMAEVGPGHTP (SEQ ID NO: 275), RGQIKLADFRLARLYSSEESR (SEQ ID NO: 276), DEQGREAELARSGPSAAGPVRLKPGLVPGL (SEQ ID NO: 277), AAVRPEQRPAARGSRV (SEQ ID NO: 278), TFPKKIQMLARDFLDEY (SEQ ID NO: 279), PETGEIQVKTFLDREQRESYELKV (SEQ ID NO: 280), PGGDSGELITDAHELGVAHPPGY (SEQ ID NO: 281), EVVGGYTWPSGNIYQGYWAQGKR (SEQ ID NO: 282), TIKNSDKNVVLEHFG (SEQ ID NO: 283), TRNSFALVPSLQRLMLRKVALKNVDSSPS (SEQ ID NO: 284), SSHYKFSKPALQSQSISLVQQS (SEQ ID NO: 285), TETVNHHYLLFQNTDLGSFHDLLR (SEQ ID NO: 286), DRASFLLTDYALSPDGSIRKATG (SEQ ID NO: 287), ERFWRNILLLSLHKGSLYPRIPGLGKE (SEQ ID NO: 288), RGRLPAGAVRTLLSQVNKVWDQSS (SEQ ID NO: 289), GHEHQPDMQKSLLRAAFFGKCFLDR (SEQ ID NO: 290), ELQYRGRELRFNLIANQHLLAPGFVSETR (SEQ ID NO: 291), EDLDANLRKLNFRLFVIRGQPAD (SEQ ID NO: 292), GHQKLPGKIHLFEAEFTQVAKKEPDG (SEQ ID NO: 293), TTPSGSAEYMASEVVEVFTDQAT (SEQ ID NO: 294), SVLREDLGQLEYKYQYAYFRMGIKHPD (SEQ ID NO: 295), PENDDLFMMPRIVDVTSLATEGG (SEQ ID NO: 296), TLDDIKEWLEDEGQVLNIQMRRTLHK (SEQ ID NO: 297), GRMSPSQFARVPGYVGSPLAAMNPK (SEQ ID NO: 298), KAHVEGDGVVEEIIRYHPFLYDRET (SEQ ID NO: 299), DGVSEEFWLVDLLPSTHYT (SEQ ID NO: 300), DSYHLYAYHEELSATVPSQWKKIG (SEQ ID NO: 301), GDQYKATDFVADWAGTFKMVFTPKDGSG (SEQ ID NO: 302), EYWKVLDGELEVAPEYPQSTARDWL (SEQ ID NO: 303), TTTSVKKEELVLSEEDFQGITPGAQ (SEQ ID NO: 304), SLTEESGGAVAFFPGNLSTSSSA (SEQ ID NO: 305), KLRTIPLSDNTIFRRICTIAKHLE (SEQ ID NO: 306), SHHTHSYQRYSHPLFLPGHRLDPPI (SEQ ID NO: 307), DVTGPHLYSIYLHGSTDKLPYVTMGS (SEQ ID NO: 308), ARLQSKEYPVIFKSIMRQRLISPQL (SEQ ID NO: 309), LHTHYDYVSALHPVSTPSKEYTSA (SEQ ID NO: 310), SDAFSGLTALPQSILLFGP (SEQ ID NO: 311), SHQIHSYQLYTHPLLHPWDHRD (SEQ ID NO: 312), STQHADLTIIDNIKEMNFLRRYK (SEQ ID NO: 313), ASATEPANDSLFSPGAANLFSTYLAR (SEQ ID NO: 314), AASAAAFPSQRTSWEFLQSLVSIKQEK (SEQ ID NO: 315), GSVLQFMPFTTVSELMKVSAMSSPKV (SEQ ID NO: 316), DKGHQFHVHPLLHSGDDLDP (SEQ ID NO: 317), NQVLASRYGIRGFSTIKIFQKGESPV (SEQ ID NO: 318), MAGPKGFQYRALYPFRRER (SEQ ID NO: 319), VTLNDMKARQKALVRERERQLA (SEQ ID NO: 320), SRLQTRKNKKLALSSTPSNIAPSD (SEQ ID NO: 321), LNTGLFRIKFKEPLENLI (SEQ ID NO: 322), SLRNNMFEISDRFIGIYKTYNITK (SEQ ID NO: 323), WCTEMKRVFGFPVHYTDVSNMS (SEQ ID NO: 324), VKQLERGEASVVDFKKNLEYAAT (SEQ ID NO: 325), STEVEPKESPHLARHRHLMKTLVKSLST (SEQ ID NO: 326), LMSNLAFADFCMRMYL (SEQ ID NO: 327), TKLKSKAPHWTNCILHEYKNLSTS (SEQ ID NO: 328), PAAGDFIRFRFFQLLRLERFF (SEQ ID NO: 329), YLSHTLGAASSFMRPTVPPPQF (SEQ ID NO: 330), ALLQNVELRRNVLVSPTPLAN (SEQ ID NO: 331), FAKGFRESDLNSWPVAPRPLLSV (SEQ ID NO: 332), GLTRISIQRAQPLPPCLPSFRPPTALQGLS (SEQ ID NO: 333), TGKPEMDFVRLAQLFARARPMGLF (SEQ ID NO: 334), and DGAWPVLLDKFVEWYKDKQMS (SEQ ID NO: 335).

7. A method for treating cancer in a subject in need thereof comprising administering to the subject an effective amount of the vaccine of claim 1.

8. The method of claim 7, further comprising sequentially, simultaneously or separately administering to the subject an effective amount of an immune checkpoint inhibitor.

9. The method of claim 8, wherein the immune checkpoint inhibitor comprises one or more of an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody; or
wherein the immune checkpoint inhibitor comprises pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189; or
wherein the immune checkpoint inhibitor is formulated for pleural, topical, parenteral, intravenous, subcutaneous, intranodal, intratumoral, intrathecal, intrapleural or intraperitoneal administration.

10. The method of claim 7, further comprising sequentially, simultaneously or separately administering to the subject an effective amount of a T-cell-engaging multi-specific antibody.

11. The method of claim 10, wherein the T-cell-engaging multi-specific antibody is a bispecific T cell engager (BiTE), a dual-affinity retargeting antibody (DART), a TandAb, a XmAb, a BITE-Fc, a 2:1 Crossmab, a duobody, a knobs-into-holes (KiH) antibody, or an IgG-scFv bispecific antibody; or
wherein the T-cell-engaging multi-specific antibody specifically targets one or more target antigens selected from among CD3, GPA33, HER2/neu, GD2, MUC16, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1); or
wherein the T-cell-engaging multi-specific antibody is formulated for pleural, topical, parenteral, intravenous, subcutaneous, intranodal, intratumoral, intrathecal, intrapleural or intraperitoneal administration.

12. The method of claim 7, further comprising sequentially, simultaneously or separately administering to the subject an effective amount of an adoptive cell therapeutic composition comprising T cells, optionally wherein the adoptive cell therapeutic composition is obtained from a donor.

13. The method of claim 12, wherein the adoptive cell therapeutic composition comprises one or more of tumor infiltrating T cells, CD8+ T cells, CD4+ T cells, delta-gamma T-cells, and alpha-beta T-cells; or
wherein the donor and the subject are the same or different; or
wherein the adoptive cell therapeutic composition is formulated for pleural, topical, parenteral, intravenous, subcutaneous, intranodal, intratumoral, intrathecal, intrapleural or intraperitoneal administration.

14. The method of claim 12, further comprising administering a cytokine to the subject.

15. The method of claim 14, wherein the cytokine is administered prior to, during, or subsequent to administration of the adoptive cell therapeutic composition; or
wherein the cytokine is selected from a group consisting of interferon a, interferon β, interferon γ, complement C5a, IL-2, TNF alpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCRIO, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

16. The method of claim 1, wherein the vaccine is formulated for pleural, topical, parenteral, intravenous, subcutaneous, intranodal, intratumoral, intrathecal, intrapleural or intraperitoneal administration.

17. The method of claim 16, wherein intratumoral administration comprises direct administration to a tumor or administration in close proximity to a tumor.

18. The method of claim 17, wherein intratumoral administration comprises delivery via an oncolytic virus or an APC vaccine.

19. The method of claim 7, wherein the cancer is a carcinoma, sarcoma, a solid non-hematopoietic cancer, or a hematopoietic cancer.

20. The method of claim 7, wherein the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, glioblastomas, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.
